(19) United States
(12) Patent Application Publication
(10) Pub. No.: US 2024/0052371 A1
(43) Pub. Date: Feb. 15, 2024

(54) PROGRAMMABLE TRANSPOSASES AND USES THEREOF

(71) Applicant: UNIVERSITAT POMPEU FABRA, Barcelona (ES)

(72) Inventors: Marc GUELL CARGOL, Barcelona (ES); Avencia SANCHEZ-MEJIAS GARCIA, Barcelona (ES); Maria PALLARES MASMITJA, Barcelona (ES); Dimitrie IVANCIC DJERMANOVIC, Barcelona (ES); Amal RAHMEH, Barcelona (ES)

(73) Assignee: UNIVERSITAT POMPEU FABRA, Barcelona (ES)

(21) Appl. No.: 18/258,039

(22) PCT Filed: Dec. 16, 2021
(86) PCT No.: PCT/EP2021/086348

§ 371 Date: Jun. 16, 2023

(30) Foreign Application Priority Data
Dec. 16, 2020 (EP) 20214696.5
Nov. 22, 2021 (EP) 21209719.0

(51) Int. Cl. C12N 15/90; C12N 15/11; C12N 9/22; C12N 9/12; C12N 15/86

(52) U.S. Cl. CPC C12N 15/907; C12N 15/11; C12N 9/22; C12N 9/1241; C12Y 207/07; C12N 15/86; C12N 2310/20; C12N 2800/80; C12N 2800/90; C12N 2740/15043

(57) ABSTRACT

The present disclosure provides efficient and precise programmable gene delivery technology based on a composition comprising (i) a first protein comprising or consisting of a site-specific DNA binding protein capable of binding and cleaving a target nucleic acid sequence; or a nucleic acid construct encoding said first protein; and (ii) a second protein comprising or consisting of a transposase; or a nucleic acid construct encoding said second protein; wherein said transposase is a modified hyperactive PiggyBac.

### TECHNICAL FIELD

The invention relates to the field of gene editing and gene therapy.

### BACKGROUND ART

Many diseases such as cancer, developmental disorders, and some infections have genetic and epigenetic aberrations in common. Gene therapy is designed to introduce genetic material into cells to target and edit the genome directly in order to correct genetically dysfunctional cells and thereby cure the associated diseases.

The gene editing toolbox has considerably expanded over the last few years has a promising tool in addition to gene therapy to repair deficient genes in order to treat disorders in subjects in need thereof.

Traditionally, gene editing is based on the design of artificial endonucleases that induce a double-strand break (DSB) into the sequence of interest in the genome1. Cells repair the DSB through one of two major pathways: Non-Homologous End-Joining (NHEJ) or Homology Directed Repair (HDR)2. Recently, editing independent on DSB has been developed. Methodologies based on directly editing DNA bases with deaminases, namely base editors (BE)3; and in situ replacing DNA bases with aid of a reverse transcriptase (RT), namely prime editors (PE)4, have become available.

However, pathological genetic defects can range from a few bases to large deletions. Base editors or prime editors only target a small number of bases, and HDR-based editing scales poorly with size5. Methodologies based on NHEJ have been developed such as Homology Independent Targeted Integration (HITI)6. This methodology has been demonstrated for insertions of several kilobases but remains inefficient for very large edits5. While HITI might work to deliver exons, it may not be efficient enough to robustly deliver cDNAs of genes such as Dystrophin (˜14 kb) or Laminin-α2 (˜9 kb). High precision CRISPR programmable transposons have been described in bacteria7,8 but are not available for mammalian cells. Previous attempts of fusing zinc fingers or Streptococcus pyogenes Cas9 (SpCas9) to the mammalian compatible piggyBac (PB) or sleeping beauty transposases delivered systems with relatively low levels of precision9-11. PB system is an attractive tool for gene therapy as efficiency scales well with size12, it is a mutation independent technology, and it works in any tissue as dependence on DNA repair mechanisms is low.

Therefore, there is still a need to develop novel systems for targeted gene delivery in mammalian cells, either in vitro, ex vivo or ex vivo.

### SUMMARY OF THE DISCLOSURE

Certain programmable transposases and their use in targeted gene editing have been disclosed in WO2020250181, which content is incorporated here by reference.

The present disclosure now provides further efficient and precise programmable gene delivery technology based on a composition comprising (i) a first protein comprising or consisting of a site-specific DNA binding protein capable of binding and cleaving a target nucleic acid sequence; or a nucleic acid construct encoding said first protein; and (ii) a second protein comprising or consisting of a transposase; or a nucleic acid construct encoding said second protein; wherein said transposase is a modified hyperactive PiggyBac. Such technology has the capability to deliver small but also large nucleic acid fragments. The inventors have tested the technology in mammalian cells and in vivo mouse liver and surprisingly achieved high efficiency (5-10%) of site directed integration in all of them.

In one embodiment, the composition comprises (i) a first protein comprising or consisting of a site-specific DNA binding protein capable of binding and cleaving a target nucleic acid sequence; or a nucleic acid construct encoding said first protein; and (ii) a second protein comprising or consisting of a transposase; or a nucleic acid construct encoding said second protein; wherein said transposase is a modified hyperactive PiggyBac, comprising one or more amino acid mutations as compared to hyperactive PiggyBac of SEQ ID NO: 9.

In one embodiment, the first protein and the second protein are fused together to form a fusion protein, optionally through a linker. In one embodiment, the first protein is fused to the C terminal end of the second protein, optionally through a linker.

In one embodiment, said transposase is a modified hyperactive PiggyBac, comprising one or more amino acid mutations to increase excision activity as compared to unmodified hyperactive PiggyBac, and/or one or more amino acid mutations to decrease DNA binding activity as compared to unmodified hyperactive PiggyBac.

In one embodiment, said one or more amino acid mutations do not consist of R372A, K375A, and D450N. In one embodiment, said one or more amino acid mutations are selected among the amino acid substitutions which increase excision activity at position of M194, D450, T560, S564 S573, S592 or F594, said position number corresponding to the amino acid number of unmodified hyperactive PiggyBac of SEQ ID NO: 9, preferably selected among the amino acid substitutions M194V and/or D450N. In one embodiment, said one or more amino acid mutations are selected among the amino acid substitutions which increase excision activity at position of M194 or D450, said position number corresponding to the amino acid number of unmodified hyperactive PiggyBac of SEQ ID NO: 9, preferably selected among the amino acid substitutions M194V and/or D450N. In one embodiment, said one or more amino acid mutations are selected among the amino acid substitutions which decrease DNA binding activity at position R275, R277, R347, R372, K375, R376, E377, and/or E380, said position number corresponding to the amino acid number of unmodified hyperactive PiggyBac of SEQ ID NO: 9, preferably selected among the amino acid substitutions R275A, R277, R347S, R372A, K375A, R376A, E377A, and/or E380A. In one embodiment, said one or more amino acid mutations are selected among the amino acid substitutions which decrease DNA binding activity at position R372, K375, R376, E377, and/or E380, said position number corresponding to the amino acid number of unmodified hyperactive PiggyBac of SEQ ID NO: 9, preferably selected among the amino acid substitutions R372A, K375A, R376A, E377A, and/or E380A.

In one embodiment, the modified hyperactive PiggyBac includes the double mutations N347S and D450N, said position number corresponding to the amino acid number of unmodified hyperactive PiggyBac of SEQ ID NO: 9. In one embodiment, the modified hyperactive PiggyBac mutation comprises one of the following amino acid substitution or combination of amino acid substitutions: R372A/K375A/R376A/D450N, K375A/R376A/E377A/E380A/D450N, R372A/K375A/R376A/E377A/E380A/D450N, M194V, R376A, E377A, E380A, M194V/R372A/K375A, S351A/R372A/K375A/R388A/D450N/W465A/S573A/M589V/S592G/F594L, R245A/R275A/R277A/R372A/W465A/M589V, R275A/325A/R372A/T560A, N347A/D450N, N347S/D450N/T560A/S573A/F594L, R202K/R275A/N347S/R372A/D450N/T560A/F594L, R275A/N347S/K375A/D450N/S592G, R275A/N347S/R372A/D450N/T560A/F594L, R275A/R277A/N347S/R372A/D450N/T560A/S564P/F594L, R245A/N347S/R372A/D450N/T560A/S564P/S573A/S592G, R277A/G325A/N347A/K375A/D450N/T560A/S564P/S573A/S592G/F594L, V34M/R275A/G325A/N347S/S351A/R372A/K375A/D450N/T560A/S564P, G325A/N347S/K375A/D450N/S573A/M589V/S592G, S230N/R277A/N347S/K375A/D450N, T43I/R372A/K375A/A411T/D450N, G325A/N347S/S351A/K375A/D450N/S573A/M589V/S592G, Y177H/R275A/G325A/K375A/D450N/T560A/S564P/S592G; the position number corresponding to the amino acid number of the hyperactive PiggyBac of SEQ ID NO: 9, typically said modified transposase has an amino acid sequence selected among any of SEQ ID NO: 2-8, 10-18 and 135-149.

In one embodiment, the composition further comprises a third protein comprising or consisting of a second transposase; or a nucleic acid construct encoding said third protein; wherein said second transposase is either an hyperactive PiggyBac with SEQ ID NO: 9, or a modified hyperactive PiggyBac with comprising one or more amino acid mutations as compared to the hyperactive PiggyBac with SEQ ID NO: 9. In one embodiment, the first, second and third proteins are fused together to form a triple fusion protein, optionally through a linker.

In one embodiment, the first protein comprises or consists of an RNA-guided nuclease or nickase, or a zinc finger nuclease. In one embodiment, said first protein is a nuclease protein comprising an active DNA cleavage domain and a guide RNA binding domain and having at least 80%, 90%, 95%, 99% or at least 100% identity to a Streptococcus pyogenes Cas9 (SpCas9) of SEQ ID NO: 31, Staphylococcus aureus Cas9 (SaCas9) of SEQ ID NO: 72, Cpf1 of SEQ ID NO: 74, Campylobacter jejuni Cas9 (CjCas9) of SEQ ID NO: 29, Streptococcus pyogenes Cas9 nickase (nCas9) of SEQ ID NO: 70, CasX of SEQ ID NO: 75, or Staphylococcus aureus Cas9 nickase of SEQ ID NO: 76; preferably wherein said first protein is a Cas9 protein selected from the group consisting of a Staphylococcus aureus Cas9 (SaCas9) of SEQ ID NO: 72 and Streptococcus pyogenes Cas9 (SpCas9) of SEQ ID NO: 31.

In one embodiment, the composition further comprises a guide RNA, and an exogenous nucleic acid for insertion in a genome.

In one embodiment, the transposase is fused to an RNA binding protein capable of binding to at least one specific RNA sequence comprised in the guide RNA; optionally wherein said RNA binding protein is an MS2 bacteriophage coat protein (MCP) and wherein the guide RNA comprises a MS2 RNA tetraloop binding sequence, preferably sharing at least 75% identity with SEQ ID NO: 153.

In one embodiment, the exogenous nucleic acid is a large DNA fragment, typically having a size between 5 kb and 25 kb, and more preferably between 8 kb and 20 kb.

In one embodiment, the composition is comprised in a nanoparticle.

The present invention also relates to a nucleic acid encoding any one of the fusion proteins disclosed herein, typically in the form of a messenger RNA (mRNA).

The present invention also relates to an in vitro method for site specific integration of an exogenous nucleic acid sequence into the genome of a cell, the method comprising delivering to the cell the composition of the invention, a guide RNA, and the exogenous nucleic acid.

The present invention also relates to the composition of the invention, a guide RNA, and an exogenous nucleic acid, for use in the treatment of a disease, by site-specific integration of the exogenous nucleic acid sequence into the genome of a cell.

### LEGENDS OF THE FIGURES

FIG. 1. Programmable transposase technology: cas9 (in red) is combined with an engineered PB transposase domain (in pink). Table of the mutants used in the experimentation with their corresponding position in PB's core model (position 563 is on the C-t which is not included in the model).

FIG. 2. A Programmable transposase dependence on variants of cas9. Nuclease cas9 and PB fusion shows better results in targeted and overall insertion as opposed to dead cas9 (dcas9) or nickase cas9 (ncas9) fusions. Blue indicated targeted insertion and yellow off-target insertion. B, Programmable transposase dependence of variants of PB. Excision enhanced mutants with reduced DNA binding present the best on-target:off-target ratio (orange). On-target insertions were performed at AAVs site (green), and TRAC site (blue). C. Testing of different linkers. Linkers length and topology does not affect significantly on-target activity of Spcas9 and PB fusions.

FIG. 3. Hershey reporter cell line: HEK293T cell line was engineered to contain a C-terminal fragment of a GFP preceded by one splicing acceptor and gRNAs target sites. A PB transposon was generated combining CAG promoter, N-terminal fragment of GFP followed by an splicing donor. In grey triangles, PB ITRs; SA: splicing acceptor; SD: splicing donor; Target: targeted insertion site; * insertion process disrupted ITR.

FIG. 4. A Programmable transposase dependence of variants of PB. Excision enhanced mutant 450 in the context of different mutations to reduce DNA binding present the best on-target. Simultaneous mutation of R372 and R376 to A is not well tolerated. Although E377 is not involved in DNA binding, the mutation to A may be beneficial to avoid a negative charge build-up in that region upon mutation of K375 and R376 to A. B R372A/K375A decrease the integration activity of PB as a result of a decrease in binding to target DNA (as observed for D450N as well). Testing of Off-target integration in progress.

FIG. 5. Double stranded breaks and programmable DNA binding domain effects in targeted insertion. Co-localization of double stranded break and PB in the insertion site is required for efficient on-targeted insertion.

FIG. 6. Sanger sequencing validation of multiple insertions (see in FIG. 2a a more comprehensive distribution measured by NGS). ITRs TTAA's are lost in the process of targeted insertion. NGG Pam is highlighted in red.

FIG. 7. Insertion activity PB K375A_R376A_E377A_E380A_D450N without cas9. In order to further investigate the targeted insertion mechanism, hyPB K375A_R376A_E377A_E380A_D450N was cloned without cas9 and its insertion efficiency was tested in comparison with hyPB WT using an RFP transposon in hek293T cells. Results show no insertion activity of this mutant without fused cas9.

FIG. 8. A Characterization of targeted insertion site using Guide-seq. Programmable Transposase generates irreversible insertion by inactivating ITR site by multiple indels. B Characterization of overall insertion site using Guide-seq. Only on-target insertions were detected on the TCR loci (upper panel). Sanger sequencing is shown for 4 clones (bottom panel).

FIG. 9. Programmable transposase characterization of insertional profiling by Guide-seq shows that hyPB mutants in combination with Cas9 performed precise transposon insertion.

FIG. 10. Benchmarking of Cas9-hyPB R372A-K375A-D450N to other targeted insertion platforms such as Cas9 induced HDR (300 bp homology arms were used).

FIG. 11. in vivo deployment of Cas9-hyPB R372A-K375A-D450N in mice liver. Relative copy number measured by qPCR is reported.

FIG. 12. Programmable transposase can be engineered with different Cas variants, such as CasX, CjCas9 Cpf1 or SaCas9, some of them achieved similar results in terms of programmable insertion at the target site as with SpCas9. Each of the Cas variant tested were targeted to the specific target region of the split GFP reporter cell line with 3 independent gRNAs.

FIG. 13. Double stranded breaks, by Cas9 and a single gRNA (gRNA-TCR1 or AAVS1-3) or by nickase Cas9 and two gRNAs targeting at nearby positions (gRNA-TCR1 and AAVS1-3), and programmable DNA binding domain (ZnF) in fusion to modified hyPB (mutants R372A-K375A-D405N) results in targeted insertion. Co-localization of double stranded break and PB in the insertion site is required for efficient on-targeted insertion. This can be achieved by nuclease Cas9 or double cut by nickase Cas9.

FIG. 14. Programmable transposase can be engineered as a dimer polypeptide of two hyPB domains and a Cas9 nuclease, resulting in better programmable insertion compared to Cas9-hyPB. Split GFP reporter cell line was used for the programmable insertion of split GFP transposon to the target site. The mutant of hyPB R372A-K375A-D450N has been used for the monomer or dimer fusion to Cas9. Conditions: 1-Negative control with only hyPB as insertion machinery; 2: Positive control of Cas9-hyPB R372A-K375A-D450N in pcDNA expression vector; 3: Positive control of Cas9-hyPB R372A-K375A-D450N in Lentivirus expression vector; 4: Cas9 nuclease fused to two units of hyPB R372A-K375A-D450N in C-terminal; 5: Cas9 nuclease fused to two units of hyPB R372A-K375A-D450N one in C-terminal and the other one in N-terminal.

FIG. 15. Several cycles of selection of cells where programmable transposition took place allowed for the selection of best mutant combinations from a library. We identified several mutants with better enrichment, and programmable insertion capacity than Cas9-hyPB R372A-K375A-D450N when fused to Cas9.

FIG. 16. On-target efficiency increases over cycles of selection. Bulk variants selected from each cycle were co-transfected with gRNA targeting AAVS1 and ½ GFP transposon into the reporter cell line. Quantity of plasmid was corrected by PB copy number to normalize for cloning efficiency.

FIG. 17. (A) On-target efficiencies of the top selected candidates. Six individual candidates were selected based on the highest on-target activity among 96 random clones selected from the last cycle. The individual on-target activities were compared to Cas9-hyPB R372A-K375A-D450N. (B) Logo showing the predominant PB residues in top on-target activity variants.

FIG. 18. Benchmarking of Cas9-hyPB R372A-K375A-D450N (FiCAT) to Homology-independent targeted insertion (HITI).

FIG. 19. Programmable insertion activity of FiCAT R372A-K375A-D450N using four different nuclease proteins. SpCas9 is used as control for programmable insertion with gRNA-TRAC-1 only (left). Each nuclease was used with three independent gRNAs (1-3) for targeted insertion in ½ GFP reporter cell line.

FIG. 20. Liver integration of minicircle luciferase transposon. Minicircle luciferase transposon, sgRNA targeting Rosa26 locus and FiCAT (Cas9-hyPB R372A-K375A-D450N) mRNA were delivered by hydrodynamic injection and luciferase signal was monitored.

FIG. 21. (a) Editing activity by CasX (left) and Cpf1 (middle). (b) Editing activity by SaCas9 (left), CjCas9 (middle). Mean % of reads with indels+/−SD is shown for two technical repeats, representative image of N=3 biological replicates. SpCas9 targeting the TRAC-1 site was used for reference (right).

FIG. 22. Increase of on-target efficiency over cycles of selection. (A) Bulk variants selected from each cycle were co-transfected with gRNA targeting AAVS1 and ½ GFP transposon into the reporter cell line. Quantity of plasmid was corrected by PB copy number to normalize for cloning efficiency. (B) Lentiviruses expressing bulk variants of each cycle were produced and used to infect reporter the cell line.

FIG. 23. Specific target integration relative to FiCAT (hyPB R372A-K375A-D450N) of single mutants isolated from bulk variants after 4 and 5 cycles of cas9_PB library enrichment co transfected with gRNA tcr1 and ½ GFP MC transposon.

FIG. 24. Programmable insertion activity of dimeric hyPB R372A-K375A-D450N fused with either SpCas9 or SaCas9 for targeted insertion in ½ GFP reporter cell line.

FIG. 25. Relative comparison of the programmable insertion activity for targeted insertion in ½ GFP reporter cell line. (A) Comparison between hyPB R372A-K375A-D450N fused with SpCas9 protein (left) and hyPB R372A-K375A-D450N fused with MCP protein with SpCas9 added separately (right). (B) Comparison between 3 hyPB mutants (R372A-K375A-D450N; R202K-R275A-N347S-R372A-D450N-T560A-F594L; and R275A-N347S-R372A-D450N-T560A-F594L) fused to MCP protein with SpCas9 added separately.

FIG. 26. Comparison of the programmable insertion activity for targeted insertion in ½ GFP reporter cell line. (A) Comparison between the co-expression of hyPB R372A-K375A-D450N and SpCas9 protein (left) and the fusion protein comprising hyPB R372A-K375A-D450N with SpCas9 protein (right). (B) Relative comparison between 3 hyPB mutants (R372A-K375A-D450N; R202K-R275A-N347S-R372A-D450N-T560A-F594L; and R275A-N347S-R372A-D450N-T560A-F594L) co-expressed with SpCas9.

FIG. 27. Relative comparison of the programmable insertion activity for targeted insertion in ½ GFP reporter cell line with the co-expression of a first fusion protein comprising SpCas and hyPB R372A-K375A-D450N, and a second fusion protein comprising MCP protein and hyPB mutants (R372A-K375A-D450N; R202K-R275A-N347S-R372A-D450N-T560A-F594L; and R275A-N347S-R372A-D450N-T560A-F594L).

FIG. 28. Relative comparison of the programmable insertion activity for targeted insertion in ½ GFP reporter cell line with the co-expression of a fusion protein comprising SpCas and hyPB R372A-K375A-D450N, and 3 hyPB mutants R372A-K375A-D450N; R202K-R275A-N347S-R372A-D450N-T560A-F594L; and R275A-N347S-R372A-D450N-T560A-F594L.

FIG. 29. Comparison of the programmable insertion activity for targeted insertion in ½ GFP reporter cell line between SpCas9 fused to a dimer of hyPB R272A-K275A-D450N (left) and SpCas9 fused to a first hyPB R272A-K275A-D450N and to a second hyPB mutant (right).

### DEFINITIONS

As used herein, the singular forms “a”, “an”, and “the” include the singular and the plural reference unless the context clearly indicates otherwise. Thus, for example, a reference to “an agent” includes a single agent and a plurality of such agents.

The terms “nucleic acid sequence” and “nucleotide sequence” may be used interchangeably to refer to any molecule composed of, or comprising, monomeric nucleotides. A nucleic acid may be an oligonucleotide or a polynucleotide. A nucleotide sequence may be a DNA, RNA, or a mix thereof. A nucleotide sequence may be chemically-modified or artificial. Nucleotide sequences include peptide nucleic acids (PNA), morpholinos and locked nucleic acids (LNA), as well as glycol nucleic acids (GNA) and threose nucleic acid (TNA). Each of these sequences is distinguished from naturally-occurring DNA or RNA by changes to the backbone of the molecule. Also, phosphorothioate nucleotides may be used. Other deoxynucleotide analogs include, without limitation, methylphosphonates, phosphoramidates, phosphorodithioates, N3′P5′-phosphoramidates and oligoribonucleotide phosphorothioates and their 2′-O-allyl analogs and 2′-O-methylribonucleotide methylphosphonates which may be used in a nucleotide of the disclosure.

The term “transgene” refers to an exogenous nucleic acid sequence, in particular an exogenous DNA or cDNA encoding a gene product. The gene product may be an RNA, peptide or protein. In addition to the coding region for the gene product (CDS), the transgene may include or be associated with one or more operational sequences to facilitate or enhance expression, such as a promoter, enhancer(s), response element(s), reporter element(s), insulator element(s), polyadenylation signal(s) and/or other functional elements. Embodiments of the disclosure may utilize any known suitable promoter, enhancer(s), response element(s), reporter element(s), insulator element(s), polyadenylation signal(s) and/or other functional elements, unless specified otherwise. Suitable elements and sequences will be well known to those skilled in the art.

The terms “polypeptide”, “peptide”, and “protein” are used interchangeably to refer to a polymer of amino acid residues. The term also applies to amino acid polymers in which one or more amino acids are chemical analogues or modified derivatives of corresponding naturally-occurring amino acids.

The term “binding protein” refers to a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to one or more molecules of the same protein to form homodimers, homotrimers, etc.; and/or it can bind to one or more molecules of a different protein or proteins. A binding protein can have more than one type of binding activity. For example, zinc finger proteins have DNA-binding, RNA-binding and protein-binding activity.

The terms “Cas9” or “Cas9 nuclease” refer to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA” or simply “gNRA”) can be engineered so as to incorporate aspects of both the crRNA and tracrRNA into a single RNA species.

Cas9 recognizes a short motif in the CRISPR repeat sequences (the PAM or protospacer adjacent motif) to help distinguish self vs. non-self. Cas9 nuclease sequences and structures are well known to those of skill in the art. Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski et al., 2013. (RNA Biol. 10(5):726-37), the entire content of which is incorporated herein by reference.

In some embodiments, a Cas9 nuclease has an inactive (e.g., an inactivated) DNA cleavage domain. A nuclease-inactivated Cas9 protein can interchangeably be referred to as a “dCas9” protein (for nuclease-“dead” Cas9). Methods for generating a Cas9 protein (or a fragment thereof) having an inactive DNA cleavage domain are known in the art (see, e.g., Jinek et al., 2012. Science. 337(6096):816-821; Qi et al., 2013. Cell. 152(5):1173-83, the entire content of each being incorporated herein by reference).

The term “zinc finger protein” refers to a protein, or a domain within a larger protein, that binds DNA in a sequence-specific manner through one or more zinc fingers, which are regions of amino acid sequences within a binding domain of the zinc finger protein whose structure is stabilized through coordination of a zinc ion. The term “zinc finger protein” is often abbreviated as “ZFP”.

The term “zinc finger nuclease” refers to an artificial restriction enzyme generated by fusing a zinc finger DNA-binding domain to a DNA-cleavage domain. Zinc finger domains can be engineered to target specific desired DNA sequences, and this enables zinc finger nucleases to target unique sequences within complex genomes. “Zinc finger nuclease” is often abbreviated as “ZFN” or “ZNP”.

The term “amino acid sequence” or “polypeptide” or “protein” as used herein, refers a polymer of amino acid residues. Unless specified, a polymer of amino acid residues can be any length.

The term “exogenous” as used herein, refers to a molecule that is not naturally present in a cell, but can be introduced into a cell by one or more genetic, biochemical or other methods. Natural presence in the cell may also be determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally functioning endogenous molecule.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally occurring episomal nucleic acid. Additional endogenous molecules can include proteins, for example, transcription factors and enzymes.

A “target sequence” or “target nucleic acid sequence” or “target site” is a sequence that defines a portion of a nucleic acid, e.g., in a genome, to which a binding molecule will bind, provided sufficient conditions for binding exist. For example, the sequence 5′-GAATTC-3′ is a target site for the EcoRI restriction endonuclease.

The term “fusion” refers to a molecule in which two or more subunit molecules are linked. In some embodiments, the link between the two is covalent; alternatively, the link between the two can be non-covalent and rely, e.g., on intermolecular interactions. The subunit molecules can be the same chemical type of molecule, or can be different chemical types of molecules.

The term “fusion protein” refers to a hybrid polypeptide which comprises protein domains from at least two different proteins. For example, one protein domain may be located at the amino-terminal (N-terminal) portion of the fusion protein or at the carboxy-terminal (C-terminal) protein, thus forming an “amino-terminal fusion protein” or a “carboxy-terminal fusion protein”, respectively. In preferred embodiments, a fusion protein is a single chain polypeptide which may be fully encoded by a nucleic acid sequence, and includes at least two protein domains directly covalently linked by peptidic bound or optionally covalently linked via a peptidic linker.

The terms “gene” or “genome” as used herein, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

The term “eukaryotic,” cells include, but are not limited to, fungal cells (such as yeast), plant cells, animal cells, mammalian cells and human cells (e.g., T-cells).

The term “linked” as used herein, refers to the juxtaposition of two or more components (such as sequence elements), in which the components are arranged such that both components function normally and allow the possibility that at least one of the components can mediate a function that is exerted upon at least one of the other components.

A “functional fragment” of a protein, polypeptide or nucleic acid is a protein, polypeptide or nucleic acid, respectively, whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions.

The term “transfect” as used herein, refers to the introduction of nucleic acids (either DNA or RNA) into eukaryotic or prokaryotic cells or organisms.

The term “cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

The term “specificity” refers to the ability to selectively bind a sequence which shares a degree of sequence identity to a selected sequence.

The terms “insertion” and “integration” refer to the addition of a nucleic acid sequence into a second nucleic acid sequence or into a genome or part thereof. The terms “specific”, “site-specific”, “targeted” and “on-targeted” in relation to insertion or integration, are used herein interchangeably to refer to the insertion of a nucleic acid into a specific site of a second nucleic acid or into a specific site of a genome or part thereof. Conversely, the terms “random”, “non-targeted” and “off-targeted” refer to non-specific and unintended insertion of a nucleic acid into an unwanted site. The terms “total” or “overall” refer to the total number of insertions.

The term “mutation” refers to a substitution of a residue within a sequence, e.g., a nucleic acid or amino acid sequence, with another residue; and/or to a deletion or insertion of one or more residues within a nucleic acid or amino acid sequence. Mutations are typically described herein by identifying the original residue followed by the position of the residue within the sequence, then the identity of the newly substituted residue. Various methods for making amino acid substitutions (mutations) provided herein are well known in the art, and are provided by, for example, Green & Sambrook, 2012 (Molecular cloning: a laboratory manual (4^{th} Ed.). Cold Spring Harbor Laboratory Press, Cold Spring Harbor, N.Y.). In preferred embodiments, the term mutation in a protein refers to an amino acid substitution.

The term “transposase” refers to an enzyme that binds to the end of a transposon and catalyzes its movement to another part of the genome by a cut-and-paste mechanism or a replicative transposition mechanism.

The term “modified” refers to a protein or nucleic acid sequence that is different than a corresponding unmodified protein or nucleic acid sequence.

The term “linker” refers to a chemical group or a molecule linking two adjacent molecules or moieties.

The terms “vector” and “plasmid” as used herein, refer to any polynucleotide that can carry, e.g., a second polynucleotide of interest, and e.g., which can transfer gene sequences to target cells. Thus, the term includes cloning, and expression vehicles, as well as integrating vectors. Particularly, the term “expression vector,” as used herein, refers to any polynucleotide capable of directing the expression of a nucleic acid. In some aspects, the terms “vector” and “plasmid” are used interchangeably with the term “nucleic acid construct.”

As used herein, the percent identity between two sequences is a function of the number of identical positions shared by the sequences (i. e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. Alternatively, the percent identity between two amino acid sequences can be determined using the Needleman and Wunsch (J. Mol, Biol. 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

The percent identity between two nucleotide amino acid sequences may also be determined using for example algorithms such as the BLASTN program for nucleic acid sequences using as defaults a word length (W) of 11, an expectation (E) of 10, M=5, N=4, and a comparison of both strands.

The terms “recombinant” or “engineered,” as used herein, refer to a protein or nucleic acid sequence that has been artificially created.

The term “subject” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal.

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent, reduce the likelihood of developing, or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### DETAILED DESCRIPTION

The present invention relates to a composition comprising
(i) a first protein comprising or consisting of a site-specific DNA binding protein capable of binding and cleaving a target nucleic acid sequence; or a nucleic acid construct encoding said first protein;
(ii) a second protein comprising or consisting of a transposase; or a nucleic acid construct encoding said second protein; and
wherein said transposase is a modified hyperactive PiggyBac, comprising one or more amino acid mutations as compared to hyperactive PiggyBac of SEQ ID NO: 9.

Current genome engineering tools, including engineered zinc finger proteins (ZFPs), transcription activator like effector nucleases (TALENs), and more recently, the RNA-guided DNA nucleases such as Cas9, effect sequence-specific DNA cleavage in a genome. This programmable cleavage can result in mutation of the DNA at the cleavage site via non-homologous end joining (NHEJ) or replacement of the DNA surrounding the cleavage site via homology-directed repair (HDR).

In one embodiment, the site-specific DNA binding protein is selected from the group comprising or consisting of RNA-guided DNA nucleases, zinc finger proteins and transcription activator like effector nucleases.

In one embodiment, the site-specific DNA binding protein is selected from the group comprising or consisting of RNA-guided DNA nucleases and zinc finger proteins.

In one embodiment, the site-specific DNA binding protein is an RNA-guided nuclease.

In one embodiment, the site-specific DNA binding protein is a Cas9 protein (e.g., without limitation, Streptococcus pyogenes Cas9 (SpCas9), Staphylococcus aureus Cas9 (SaCas9), or Campylobacter jejuni Cas9 (CjCas9); some other suitable examples will be described below), or a variant thereof (e.g., nickase Cas9 (nCas9) or dead Cas9 (dCas9)), a Cas12a protein, a Cas12b protein, a Cpf1 protein, or a CasX protein, including variants and functional fragments thereof.

In one embodiment, the site-specific DNA binding protein is a Cas9 protein, including variants and functional fragments thereof.

The CRISPR-Cas9 system is a highly effective tool for inactivating or modifying genes via sequence-specific double-strand breaks (DSBs). These DSBs are recognized by the cellular DNA damage response machinery and can be repaired by endogenous DSB repair pathways. The predominant repair pathway is non-homologous end joining (NHEJ), which often results in small insertions and/or deletions that can create frameshift mutations and disrupt the function of genes. This pathway can be exploited to generate genetic knockout mutations. Alternatively, in the presence of repair templates (such as, e.g., the nucleic acid construct comprising or consisting of a transgene encoding the laminin-α2 protein, functional variant or fragment thereof), the damage can be repaired seamlessly by homology-directed repair (HDR). However, despite remarkable progress, HDR-mediated genome editing to introduce precise genetic modifications is much less efficient than NHEJ-mediated gene disruption. Furthermore, large multi-kb replacements by the HDR pathways results challenging and requires selection and/or large population cell sorting. Consequently, the major applications for the HDR pathways are currently limited to the local replacement of key regions within genes, but not of large, full-length genes. As explained above, the present invention remedies this deficiency.

In one embodiment, the Cas9 protein comprises (i) an active DNA cleavage domain and (ii) a guide RNA binding domain.

Among the known Cas9 proteins, the S. pyogenes Cas9 protein has been widely used as a tool for genome engineering. This Cas9 protein is a large, multi-domain protein containing two distinct nuclease domains.

In one embodiment, the Cas9 protein is selected from the group comprising or consisting of the Cas9 protein from Corynebacterium ulcerans (NCBI Refs: NC_015683.1, NC_017317.1) with SEQ ID NO: 19); Corynebacterium diphtheria (NCBI Refs: NC_016782.1, NC_016786.1) with SEQ ID NO: 20; Spiroplasma syrphidicola (NCBI Ref: NC_021284.1) with SEQ ID NO: 21; Prevotella intermedia (NCBI Ref: NC_017861.1) with SEQ ID NO: 22; Spiroplasma taiwanense (NCBI Ref: NC_021846.1) with SEQ ID NO: 23; Streptococcus iniae (NCBI Ref: NC_021314.1) with SEQ ID NO: 24; Belliella baltica (NCBI Ref: NC_018010.1) with SEQ ID NO: 25; Psychroflexus torquisi (NCBI Ref: NC_018721.1) with SEQ ID NO: 26; Streptococcus thermophilus (NCBI Ref: YP_820832.1) with SEQ ID NO: 27; Listeria innocua (NCBI Ref: NP_472073.1) with SEQ ID NO: 28; Campylobacter jejuni (CjCas9) (NCBI Ref: YP_002344900.1) with SEQ ID NO: 29 (encoded by SEQ ID NO: 81); Neisseria meningitidis (NCBI Ref: YP_002342100.1) with SEQ ID NO: 30; Staphylococcus aureus (SaCas9) with SEQ ID NO: 72 (encoded by SEQ ID NO: 77); and Streptococcus pyogenes (SpCas9) (NCBI Ref: NC_017053.1) with SEQ ID NO: 31.

In one embodiment, when referring herein to the wild-type Cas9 protein, said wild-type Cas9 protein corresponds to Cas9 from Streptococcus pyogenes (spCas9) with SEQ ID NO: 31, unless specified otherwise.

In one embodiment, the Cas9 protein may be a “Cas9 variant”. A “Cas9 variant”, as used herein, is a protein sharing homology to a Cas9 protein as described herein, and includes fragments thereof.

In one embodiment, the Cas9 variant can be at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a wild-type Cas9 protein with SEQ ID NO: 31, or to any other Cas9 protein with SEQ ID NOs: 19-30 or 72.

In one embodiment, the Cas9 variant comprises the amino acid sequence of a Cas9 protein with one or several amino acid substitutions. For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand.

Mutations within these subdomains can silence the nuclease activity of Cas9. For example, the substitutions D10A and H841A are known to completely inactivate the nuclease activity of the S. pyogenes Cas9 protein with SEQ ID NO: 31, resulting in a dead Cas9 (dCas9) that still retains its ability to bind DNA in a sgRNA-programmed manner. In principle, when fused to another protein or domain, dCas9 can target that protein to virtually any DNA sequence simply by co-expression with an appropriate sgRNA. In one embodiment, the dCas9 protein is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 66. In one embodiment, the dCas9 protein comprises or consists of an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 71.

As to Cas9 nickase (nCas9), it is a variant of Cas9 nuclease differing by a point mutation (D10A) in the RuvC nuclease domain, which enables it to nick, but not cleave, DNA. In one embodiment, the nCas9 protein is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 65. In one embodiment, the nCas9 protein comprises or consists of an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 70. In some embodiments, the SaCas9 nickase (SanCas9) is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 80. In some embodiments, the SaCas9 nickase (SanCas9) comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 76.

In one embodiment, the Cas9 variant comprises a fragment of Cas9, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of a wild-type Cas9 protein with SEQ ID NO: 31, or of any other Cas9 protein with SEQ ID NOs: 19-30 or 72.

In one embodiment, the Cas9 variant comprises only one of a DNA cleavage domain or a guide RNA binding domain.

In one embodiment, an exemplary Cas9 variant is humanized Cas9 (hCas9) or a variant or functional fragment thereof. As used herein, the term “humanized Cas9” or “hCas9” refers to a sequence-optimized Cas9 protein for human cells.

In one embodiment, the hCas9 protein is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 64. In one embodiment, the hCas9 protein comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 69.

In one embodiment, the site-specific DNA binding protein is a cpf1 protein. In one embodiment, the cpf1 protein is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 78. In one embodiment, the cpf1 protein comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 74.

In one embodiment, the site-specific DNA binding protein is a CasX protein. In one embodiment, the CasX is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 79. In one embodiment, the CasX comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 75.

As will be further detailed below, certain aspects of the disclosure are also directed to vectors or plasmids (e.g., expression vectors, packaging vectors, etc.) comprising a nucleic acid construct encoding the site-specific DNA binding protein, in particular the RNA-guided nuclease, in particular any of the Cas9 proteins described herein; said vectors or plasmids being preferably suitable for expression in a host cell, e.g., mammalian cells, yeast cells, insect cells, plant cells, fungal cells, or algal cells.

In one embodiment, the site-specific DNA binding protein is a zinc finger protein (ZFP).

Zinc finger proteins are proteins that can bind to DNA in a sequence-specific manner. ZFP are unevenly distributed in eukaryotes. ZFP have been identified that are involved in DNA recognition, RNA binding, and protein binding. Certain classifications for zinc finger proteins are based on “fold groups” in view of the overall shape of the protein backbone in the folded domain. The most common “fold groups” of zinc fingers are the C2H2 or Cys2His2-like (the “classic zinc finger”), treble clef, and zinc ribbon. Representative motifs characterizing these proteins are disclosed in Table 1 of Li & Liu, 2020 (Int J Mol Sci. 21(4):1361), which Table is herein incorporated by reference.

The ZFP can be any ZFP, variant or functional fragment thereof, that can bind to a specific genomic DNA sequence in a genome. Non-limiting examples of ZFPs include ZFPs comprising a fold group or zinc finger motif selected from C2H2, gag knuckle, treble clef, zinc ribbon, Zn2/Cys6-like, or TAZ2 domain-like, or any combination thereof. In one embodiment, the ZFP is a C2H2 zinc finger protein.

In one embodiment, the ZFP is an engineered ZFP. Engineered zinc finger arrays can be fused to a DNA cleavage domain (usually the cleavage domain of FokI) to generate zinc finger nucleases. Such zinc finger-FokI fusions have become useful reagents for manipulating genomes.

The ZFP can comprise 2, 3, 4, 5, 6, 7, 8, 9, 10, 11, 12, or more zinc finger domains. The ZFP can comprise from 2 to 12, from 2 to 10, from 2 to 8, from 3 to 8, from 4 to 8, or from 5 to 8 zinc finger domains. In one embodiment, the ZFP comprises 6 zinc finger domains.

A common modular assembly process involves combining separate zinc fingers that can each recognize a 3-basepair DNA sequence to generate 3-finger, 4-, 5-, or 6-finger arrays that recognize target sites ranging from 9 basepairs to 18 basepairs in length. Another method uses 2-finger modules to generate zinc finger arrays with up to six individual zinc fingers.

In one embodiment, the binding domain of the ZFP can be engineered to bind to a sequence of interest. An engineered zinc finger binding domain can have improved binding specificity, compared to a naturally-occurring ZFP.

In one embodiment, exemplary nucleic acid sequences encoding the ZFP comprise or consists of SEQ ID NO: 32, SEQ ID NO: 34, SEQ ID NO: 36, or SEQ ID NO: 38. In one embodiment, exemplary amino acid sequences encoded by these sequences comprise or consists of SEQ ID NO: 33, SEQ ID NO: 35, SEQ ID NO: 37, or SEQ ID NO: 39.

In one embodiment, the ZFP comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to any one of SEQ ID NOs: 33, 35, 37 or 39.

In one embodiment, the ZFP does not have a Gal4 DNA binding domain. Gal4 binds to CGG-N₁₁-CCG, where N can be any base. This protein is a positive regulator for the gene expression of the galactose-induced genes such as GAL1, GAL2, GAL7, GAL10, and MEL1 which code for the enzymes used to convert galactose to glucose. It recognizes a 17-base pair sequence in the upstream activating sequence (UAS-G) of these genes. Therefore, Gal4 recognizes a short and very frequent sequence in the genome, thus not being site-specific. In one embodiment, the ZFP has a Gal4 DNA binding domain engineered to be site-specific.

As will be further detailed below, certain aspects of the disclosure are directed to vectors or plasmids (e.g., expression vectors, packaging vectors, etc.) comprising a nucleic acid construct encoding the site-specific DNA binding protein, in particular the ZFP described herein; said vectors or plasmids being preferably suitable for expression in a host cell, e.g., mammalian cells, yeast cells, insect cells, plant cells, fungal cells, or algal cells.

According to the invention, the second protein comprises or consists of a transposase.

Transposons are chromosomal segments that can undergo transposition, e.g., DNA that can be translocated as a whole in the absence of a complementary sequence in the host DNA. Transposons can be used to perform long-range DNA engineering in human cells. Common transposon systems used in mammalian cells include, without limitation, Sleeping Beauty (SB), which was reconstructed from inactive transposons, and PiggyBac (PB), isolated from the moth Trichoplusia. PiggyBac has higher transposition activity than SB and it can be excised scarlessly.

Native DNA transposons typically contain a single gene coding for a transposase protein, which is flanked by Inverted Terminal Repeats (ITRs) that carry transposase binding sites. During their transposition, the transposase protein recognizes these ITRs to catalyze excision and subsequent reintegration of the element elsewhere in a random manner. Moreover, some of these transposons can be adapted for use in gene therapy protocols, employing them as bi-component systems, in which a plasmid contains an expression cassette where a DNA sequence of interest, placed between the transposon ITRs, can be introduced into a host genome directed by a co-transfected plasmid containing the sequence encoding the transposase enzyme or its mRNA synthesized in vitro. According to the disclosure, a transposon-based system is used to efficiently mediate stable integration and persistent expression of transgenes in a cell, as therapeutic genes.

A transposase or modified transposase of the disclosure can be any transposase that can insert an exogenous nucleic acid into a specific site of a genome. Some aspects of this disclosure provide transposase fusion proteins that are designed using the methods and strategies described herein. Some embodiments of this disclosure provide nucleic acids encoding such transposases or modified transposases and/or fusion proteins comprising the same. Some embodiments of this disclosure provide plasmids or expression vectors comprising such nucleic acid constructs encoding transposases or modified transposases and/or fusion proteins comprising the same.

Non-limiting examples of transposases include Frog Prince, Sleeping Beauty, hyperactive Sleeping Beauty, PiggyBac, and hyperactive PiggyBac.

In one embodiment, the transposase is a hyperactive PiggyBac transposase. In some embodiments, the transposase is the hyperactive PiggyBac transposase corresponding to SEQ ID NO: 9 or as encoded by SEQ ID NO: 67 (referred in this disclosure also as hyPB or simply as PB).

In one embodiment, the transposase is a modified hyperactive PiggyBac transposase.

As used herein, “modified hyperactive PiggyBac transposase” refers to a transposase comprising one or more amino acid substitutions, typically no more than 1, 2, 3, 4, 5, 6, 7, 8, 9, or 10 amino acid substitutions, as compared to the wild-type hyperactive PiggyBac transposase with SEQ ID NO: 9. More specifically, a modified hyperactive PiggyBac comprises (i) one or more amino acid substitutions to increase excision activity as compared to the wild-type hyperactive PiggyBac transposase, and/or (ii) one or more amino acid substitutions to decrease DNA binding activity as compared to the wild-type hyperactive PiggyBac transposase. In one embodiment, the modified hyperactive PiggyBac transposase comprises an amino acid sequence at least 85%, at least 90%, at least 95%, at least 96%, at least 97%, at least 98%, or at least 99% identical to the sequence set forth in SEQ ID NO: 9.

In some embodiments, the one or more mutations to the hyperactive PiggyBac transposase do not consist of a triple mutation R372A/K375A/D450N, said position numbers corresponding to the amino acid numbers of unmodified hyperactive PiggyBac of SEQ ID NO:9.

In some embodiment, the modified hyperactive PiggyBac comprises one or more amino acid mutations to increase excision activity.

In some embodiment, the modified hyperactive Piggybac comprises one or more amino acid mutations to increase excision activity selected among the amino acid mutations within the region defined by the amino acid position numbers [194-200], [214-222], [434-442] or [446-456], for example amino acid substitution at the position D198, D201, R202, M212 and/or S213; said position number corresponding to the amino acid number of unmodified hyperactive Piggybac of SEQ ID NO:9.

In some embodiment, the modified hyperactive Piggybac comprises one or more amino acid mutations to increase excision activity selected among the amino acid mutations at positions 450, 560, 564, 573, 589, 592, and/or 594; said position number corresponding to the amino acid number of unmodified hyperactive Piggybac of SEQ ID NO: 9.

In some embodiment, the modified hyperactive PiggyBac comprises one or more amino acid mutations to increase excision activity selected among the amino acid mutations at position of M194 and/or D450, said position number corresponding to the amino acid number of unmodified hyperactive Piggybac of SEQ ID NO:9, preferably the amino acid substitution selected among M194V and/or D450N.

In some embodiment, the modified hyperactive PiggyBac comprises one or more amino acid mutations to decrease DNA binding activity.

In some embodiment, the modified hyperactive PiggyBac comprises one or more amino acid mutations to decrease DNA binding activity selected among the amino acid mutations at positions 254, 275, 277, 347, 372, 375, and/or 465; said position number corresponding to the amino acid number of unmodified hyperactive Piggybac of SEQ ID NO: 9.

In some embodiment, the modified hyperactive PiggyBac comprises one or more amino acid mutations to decrease DNA binding activity selected among R275, N347, R372, K375, R376, E377, and E380, said position number corresponding to the amino acid number of unmodified hyperactive Piggybac of SEQ ID NO:9.

In some embodiment, the modified hyperactive PiggyBac comprises one or more amino acid mutations to decrease DNA binding activity selected among R372, K375, R376, E377, and E380, said position number corresponding to the amino acid number of unmodified hyperactive Piggybac of SEQ ID NO:9, preferably selected among the amino acid substitutions R372A, K375A, R376A, E377A, and/or E380A.

In some embodiment, the modified hyperactive PiggyBac comprises one or more amino acid mutations to decrease DNA binding activity selected among N347, R372, and K375, said position number corresponding to the amino acid number of unmodified hyperactive Piggybac of SEQ ID NO:9, preferably selected among the amino acid substitutions N347S, N347A, R372A, K375A, more preferably selected among the amino acid substitutions N347S, N347A.

In some embodiment, the modified hyperactive Piggybac comprises one or more amino acid mutations to increase excision activity, as defined above; and one or more amino acid mutations to decrease DNA binding activity, as defined above.

In some embodiment, the modified hyperactive Piggybac includes at least one amino acid substitution to increase excision activity at position D450, and at least two amino acid substitutions to decrease DNA binding activity at positions N347, R372 and K375, preferably said modified transposase of hyperactive Piggybac includes the double mutations N347S and D450N or triple mutations D450N, R372A and K375A, said position number corresponding to the amino acid number of unmodified hyperactive Piggybac of SEQ ID NO:9. In a more preferred embodiment, the modified transposase of hyperactive Piggybac includes the double mutations N347S and D450N, said position number corresponding to the amino acid number of unmodified hyperactive Piggybac of SEQ ID NO:9.

In some embodiment, the modified hyperactive Piggybac as disclosed in the previous embodiments further comprises at least one mutation in the region defined by the amino acid position numbers [158-169], for example A166S; and/or at least one mutation at position Y527, R518, K525, N463.

Typically, said modified hyperactive Piggybac comprises an amino acid sequence having at least 85%, at least 90%, at least 95% identity, or 100% identity to modified hyperactive Piggybac of SEQ ID NO: 1.

In some embodiments, said modified hyperactive Piggybac is a variant of the hyperactive Piggybac of SEQ ID NO:9 with one or more amino acid substitutions, typically with no more than 1, 2, 3, 4, 5, 6, 7, 8, 9 or 10 amino acid substitutions as compared to SEQ ID NO:9.

In some embodiments, said modified hyperactive Piggybac further comprises one or more of the following amino acid mutations at positions 34, 43, 117, 202, 230, 245, 268, 275, 277, 287, 290, 315, 325, 341, 346, 347, 350, 351, 356, 357, 388, 409, 411, 412, 432, 447, 460, 461, 465, 517, 560, 564, 571, 573, 576, 586, 587, 589, 592, and/or 594, the position number corresponding to the amino acid number of the hyperactive PiggyBac sequence (SEQ ID NO: 9).

In some embodiments, said modified hyperactive PiggyBac comprises the following mutations or combination of mutations: V34M, T43I, Y177H, R202K, S230N, R245A, D268N, K287A, K290A, K287A/K290A, R315A, G325A, R341A, D346N, N347A, N347S, T350A, S351E, S351P, S351A, K356E, N357A, R388A, K409A, A411T, K412A, K432A, D447A, D447N, D450N, R460A, K461A, W465A, S517A, T560A, S564P, S571N, S573A, K576A, H586A, I587A, M589V, S592G, or F594L, D450N/R372A/K375A, R275A/R277A, K409A/K412A, R460A/K461A, R275A/R277A/N347S/K375A/T560A/S573A/M589V/S592G and R245A/R275A/R277A/R372A/W465A.

In some embodiments, said modified hyperactive PiggyBac comprises the following amino acid substitution or combination of amino acid substitutions: R372A/K375A/D450N, R372A/K375A/R376A/D450N, K375A/R376A/E377A/E380A/D450N, R372A/K375A/R376A/E377A/E380A/D450N, M194V, M194V/R372A/K375A, S351A/R372A/K375A/R388A/D450N/W465A/S573A/M589V/S592G/F594L, R245A/R275A/R277A/R372A/W465A/M589V, R275A/325A/R372A/T560A, N347A/D450N, N347S/D450N/T560A/S573A/F594L, R202K/R275A/N347S/R372A/D450N/T560A/F594L,R275A/N347S/K375A/D450N/S 592G, R275A/N347S/R372A/D450N/T560A/F594L, R275A/R277A/N347S/R372A/D450N/T560A/S564P/F594L, R245A/N347S/R372A/D450N/T560A/S564P/S573A/S592G, R277A/G325A/N347A/K375A/D450N/T560A/S564P/S573A/S592G/F594L, V34M/R275A/G325A/N347S/S351A/R372A/K375A/D450N/T560A/S564P, G325A/N347S/K375A/D450N/S573A/M589V/S592G, S230N/R277A/N347S/K375A/D450N, T43I/R372A/K375A/A411T/D450N, G325A/N347S/S351A/K375A/D450N/S573A/M589V/S592G, Y177H/R275A/G325A/K375A/D450N/T560A/S564P/S592G, the position number corresponding to the amino acid number of the hyperactive PiggyBac sequence (SEQ ID NO: 9).

Very preferred modified hyperactive PiggyBac transposases for use according to the present disclosure include modified hyperactive PiggyBac comprising the following combination of amino acid substitutions: R372A/K375A/D450N, S351A/R372A/K375A/R388A/D450N/W465A/S573A/M589V/S592G/F594L, R245A/R275A/R277A/R372A/W465A/M589V, N347A/D450N, N347S/D450N/T560A/S573A/F594L, R202K/R275A/N347S/R372A/D450N/T560A/F594L,R275A/N347S/K375A/D450N/S 592G, R275A/N347S/R372A/D450N/T560A/F594L, R275A/R277A/N347S/R372A/D450N/T560A/S564P/F594L, R245A/N347S/R372A/D450N/T560A/S564P/S573A/S592G, R277A/G325A/N347A/K375A/D450N/T560A/S564P/S573A/S592G/F594L, V34M/R275A/G325A/N347S/S351A/R372A/K375A/D450N/T560A/S564P, G325A/N347S/K375A/D450N/S573A/M589V/S592G, S230N/R277A/N347S/K375A/D450N, T43I/R372A/K375A/A411T/D450N, G325A/N347S/S351A/K375A/D450N/S573A/M589V/S592G, Y177H/R275A/G325A/K375A/D450N/T560A/S564P/S592G and R275A/325A/R372A/T560A, the position number corresponding to the amino acid number of the hyperactive PiggyBac sequence (SEQ ID NO: 9).

In some embodiments, said modified hyperactive PiggyBac comprises the following amino acid substitution or combination of amino acid substitutions: R245A/R275A/R277A/R372A/W465A/M589V, R275A/325A/R372A/T560A, N347A/D450N, N347S/D450N/T560A/S573A/F594L, R202K/R275A/N347S/R372A/D450N/T560A/F594L, R275A/N347S/K375A/D450N/S592G, R275A/N347S/R372A/D450N/T560A/F594L, R275A/R277A/N347S/R372A/D450N/T560A/S564P/F594L, R245A/N347S/R372A/D450N/T560A/S564P/S573A/S592G, R277A/G325A/N347A/K375A/D450N/T560A/S564P/S573A/S592G/F594L, G325A/N347S/K375A/D450N/S573A/M589V/S592G, S230N/R277A/N347S/K375A/D450N, G325A/N347S/S351A/K375A/D450N/S573A/M589V/S592G, Y177H/R275A/G325A/K375A/D450N/T560A/S564P/S592G, the position number corresponding to the amino acid number of the hyperactive PiggyBac sequence (SEQ ID NO: 9).

In a more preferred embodiment, said modified hyperactive PiggyBac comprising the following combination of amino acid substitutions: N347A/D450N, N347S/D450N/T560A/S573A/F594L, R202K/R275A/N347S/R372A/D450N/T560A/F594L, R275A/N347S/K375A/D450N/S592G, R275A/N347S/R372A/D450N/T560A/F594L, R275A/R277A/N347S/R372A/D450N/T560A/S564P/F594L, R245A/N347S/R372A/D450N/T560A/S564P/S573A/S592G, R277A/G325A/N347A/K375A/D450N/T560A/S564P/S573A/S592G/F594L, G325A/N347S/K375A/D450N/S573A/M589V/S592G, S230N/R277A/N347S/K375A/D450N, G325A/N347S/S351A/K375A/D450N/S573A/M589V/S592G, Y177H/R275A/G325A/K375A/D450N/T560A/S564P/S592G, the position number corresponding to the amino acid number of the hyperactive PiggyBac sequence (SEQ ID NO: 9).

In some embodiments, said modified transposase has an amino acid sequence selected among any of SEQ ID NO: 1-8, 10-18 and 135-149.

In some embodiments, said modified transposase has an amino acid sequence selected among any of SEQ ID NO: 1-8 and 10-18.

In some embodiments, said modified transposase has an amino acid sequence selected among any of SEQ ID NO: 90-99.

In some embodiments, said modified transposase has an amino acid sequence selected among any of SEQ ID NO: 135-149. In some embodiments, said modified transposase has an amino acid sequence selected among any of SEQ ID NO: 135-140. In some embodiments, said modified transposase has an amino acid sequence selected among any of SEQ ID NO: 141-149.

In some embodiments, the modified transposase can comprise one or more mutations relative to hyPB that are involved in the conserved catalytic triad, e.g., at amino acid 268 and/or 346 (e.g., D268N and/or D346N) corresponding to the amino acid numbering of SEQ ID NO: 9 or SEQ ID NO: 11.

In some embodiments, the modified transposase can comprise one or more mutations relative to hyPB that are critical for excision, e.g., at amino acid 287, 287/290 and/or 460/461 (e.g., K287A, K287A/K290A, and/or R460A/K461A) corresponding to the amino acid numbering of SEQ ID NO: 9 or SEQ ID NO: 12.

In some embodiments, the modified transposase can comprise one or more mutations relative to hyPB that are involved in target joining, e.g., at amino acid 351, 356, and/or 379 (e.g., S351E, S351P, S351A, and/or K356E) corresponding to the amino acid numbering of SEQ ID NO: 9 or SEQ ID NO: 13.

In some embodiments, the modified transposase can comprise one or more mutations relative to hyPB that are critical for integration, e.g., at amino acid 560, 564, 571, 573, 589, 592, and/or 594 (e.g., T560A, S564P, S571N, S573A, M589V, 5592G, and/or F594L) corresponding to the amino acid numbering of SEQ ID NO: 9 or SEQ ID NO: 14.

In some embodiments, the modified transposase can comprise one or more mutations relative to hyPB that are involved in alignment, e.g., at amino acid 325, 347, 350, 357 and/or 465 (e.g., G325A, N347A, N347S, T350A and/or W465A) corresponding to the amino acid numbering of SEQ ID NO: 9 or SEQ ID NO: 15.

In some embodiments, the modified transposase can comprise one or more mutations relative to hyPB that are well conserved, e.g., at amino acid 576 and/or 587 (e.g., K576A and/or I587A) corresponding to the amino acid numbering of SEQ ID NO: 9 or SEQ ID NO: 16.

In some embodiments, the modified transposase can comprise one or more mutations relative to hyPB that are involved in Zn2+ binding, e.g., 586 (e.g., H586A) corresponding to the amino acid numbering of SEQ ID NO: 9 or SEQ ID NO: 17.

In some embodiments, the programmable transposase can comprise one or more mutations relative to hyPB that are involved in integration e.g., 315, 341, 372, and/or 375 (e.g., R315A, R341A, R372A, and/or K375A) corresponding to the amino acid numbering of SEQ ID NO: 9 or SEQ ID NO: 18.

In some embodiments, the modified hyperactive PiggyBac comprises an amino acid sequence at least 85%, at least 90%, at least 95%, at least 96%, at least 97%, at least 98%, or at least 99% identical to the sequence set forth in SEQ ID NO: 9. In some embodiments, the modified hyperactive PiggyBac is selected for its high specificity of DNA integration into a genome compared to hyperactive PiggyBac. In some embodiments, the modified hyperactive PiggyBac comprises an amino acid sequence having one or more of the modifications disclosed herein relative to SEQ ID NO: 9, 10, 11, 12, 13, 14, 15, 16, 17, or 18, and retains at least 80%, at least 85%, at least 90%, at least 95%, at least 96%, at least 97%, at least 98%, or at least 99% identical to the sequence set forth in SEQ ID NO: 1, 2, 3, 4, 5, 6, 7, 8, 9, 10, 11, 12, 13, 14, 15, 16, 17, or 18, respectively.

In some embodiments, the hyperactive PiggyBac transposase is encoded by a nucleic acid sequence having at least 85%, 90%, 95%, 96%, 97%, 98%, 99%, or 100% sequence identity to SEQ ID NO: 67. In some embodiments, the SB100 transposase is encoded by a nucleic acid sequence having at least 85%, 90%, 95%, 96%, 97%, 98%, 99%, or 100% sequence identity to SEQ ID NO: 68.

In some embodiments, the SB100 transposase comprises an amino acid sequence having at least 85%, 90%, 95%, 96%, 97%, 98%, 99%, or 100% sequence identity to SEQ ID NO: 73.

In some embodiments, the modified transposase is a modified Sleeping Beauty transposase comprising one or more mutations. In some embodiments, the one or more mutations in Hyper Active Sleeping Beauty Transposase or SB100 corresponds to: L25F, R36A, I42K, G59D, I212K, N245S, K252A and Q271L of SEQ ID NO: 9 or SEQ ID NO: 73.

In certain embodiments, the modified transposase is not a Himar1C9 mutant.

Certain aspects of the disclosure are directed to a vector or a plasmid (e.g., an expression vector or a packaging vector) comprising a nucleic acid construct comprising a transposase or a modified transposase of the disclosure suitable for expression in a host cell, e.g., mammalian cells, yeast cells, insect cells, plant cells, fungal cells, or algal cells. In some embodiments, the modified transposase is expressed as a fusion protein with a Cas9. In some embodiments, the modified transposase is co-expressed with a Cas9 from separate vectors, but delivered to the same cell. In some embodiments, the modified transposase or the fusion protein comprising the same is packaged in a lentivirus particle for delivery to a cell.

As shown in the examples, newly developed hyperactive PiggyBac transposase mutations library have been used to identify modified hyperactive PiggyBac which perform specific targeted transpositions. Modified hyperactive PiggyBac with positive targeted transposition were identified using such library.

In some embodiments, the modified hyperactive PiggyBac transposase can comprise a mutation of one or more of amino acids selected from amino acid: 245, 275, 277, 325, 347, 351, 372, 375, 388, 450, 465, 560, 564, 573, 589, 592, 594 corresponding to the amino acid numbering of SEQ ID NO: 9.

In some embodiments, the modified hyperactive PiggyBac transposase mutation can comprise one or more of the amino acid modifications selected from: R245A, R275A, R277A, R275A/R277A, G325A, N347A, N347S, S351E, S351P, S351A, R372A, K375A, R388A, D450N, W465A, T560A, S564P, S573A, M589V, S592G, or F594L corresponding to the amino acid numbering of SEQ ID NO: 9.

In an embodiment, the modified hyperactive PiggyBac transposase comprises the amino acid modification D450N corresponding to the amino acid numbering of SEQ ID NO: 9.

In an embodiment, the modified hyperactive PiggyBac transposase correspond to SEQ ID NO:1 and comprises the amino acid modifications R372A, K375A and D450.

In an embodiment, the modified hyperactive PiggyBac transposase comprises the amino acid modifications R245A and D450, corresponding to the amino acid numbering of SEQ ID NO: 9.

In an embodiment, the modified hyperactive PiggyBac transposase comprises the amino acid modifications R245A, G325A, and S573P, corresponding to the amino acid numbering of SEQ ID NO: 9.

In an embodiment, the modified hyperactive PiggyBac transposase comprises the amino acid modifications R245A, G325A, D450 and S573P, corresponding to the amino acid numbering of SEQ ID NO: 9.

In an embodiment, the modified hyperactive PiggyBac transposase comprises the amino acid modification N347S or N347A, corresponding to the amino acid numbering of SEQ ID NO: 9.

In an embodiment, the modified hyperactive PiggyBac transposase comprises the amino acid modifications N347S and D450N, corresponding to the amino acid numbering of SEQ ID NO: 9.

In another, the modified hyperactive PiggyBac transposase comprises the amino acid modifications N347A and D450N, corresponding to the amino acid numbering of SEQ ID NO: 9. In one embodiment, this modified hyperactive PiggyBac transposase comprises the amino acid sequence of SEQ ID NO: 137.

As said before, herein provided are modified hyperactive PiggyBac transposases which can be fused to the elements disclosed herein but can also be used alone or in combination with different elements. Said transposases have been generated by the inventors. Thus, modified hyperactive PiggyBac transposases are provided which comprises the amino acid sequence SEQ ID NO: 9, wherein:
amino acid at position 34 is V or M,
amino acid at position 43 is T or I,
amino acid at position 177 is Y or H,
amino acid at position 202 is R or K,
amino acid at position 230 is S or N,
amino acid at position 245 is A,
amino acid at position 268 is D or N,
amino acid at position 277 is R or A,
amino acid at position 275 is R or A,
amino acid at position 277 is R or A,
amino acid at position 325 is A or G,
amino acid at position 347 is S, or A,
amino acid at position 351 is E, P or A,
amino acid at position 372 is R or A,
amino acid at position 375 is K or A,
amino acid at position 388 is R or A,
amino acid at position 409 is K or A,
amino acid at position 411 is A or T,
amino acid at position 412 is K or A,
amino acid at position 450 is D or N,
amino acid at position 460 is R or A,
amino acid at position 465 is W or A,
amino acid at position 517 is S or A,
amino acid at position 560 is T or A,
amino acid at position 564 is P or S,
amino acid at position 571 is S or N,
amino acid at position 573 is S or A,
amino acid at position 576 is K or A,
amino acid at position 586 is H or A,
amino acid at position 587 is I or A,
amino acid at position 589 is M or V,
amino acid at position 592 is G or S, and/or,
amino acid at position 594 is L or F.

The present disclosure also relates to the modified hyperactive PiggyBac transposases provided herein for use as medicaments, particularly in gene therapy, ex vivo or in vivo.

In one embodiment, the first protein comprising or consisting of the site-specific DNA binding protein capable of binding and cleaving a target nucleic acid sequence (as described above), and the second protein comprising or consisting of a transposase (as described above), are fused together to form a fusion protein, either directly or indirectly via a linker.

Any embodiments relating to the site-specific DNA binding protein on one hand, and to the transposase on the other hand, apply mutatis mutandis in the case of the fusion protein described herein.

Hence, in one embodiment, the fusion protein comprises or consists of:
(i) a first protein comprising or consisting of an RNA-guided DNA nuclease, a zinc finger protein or a transcription activator like effector nuclease, as described above, and
(ii) a second protein comprising or consisting of a transposase, said transposase being a modified hyperactive PiggyBac comprising one or more amino acid mutations as compared to hyperactive PiggyBac of SEQ ID NO: 9, as described above.

In one embodiment, the fusion protein comprises or consists of:
(i) a first protein comprising or consisting of an RNA-guided DNA nuclease or zinc finger protein, as described above, and
(ii) a second protein comprising or consisting of a transposase, said transposase being a modified hyperactive PiggyBac comprising one or more amino acid mutations as compared to hyperactive PiggyBac of SEQ ID NO: 9, as described above.

In one embodiment, the fusion protein comprises or consists of:
(i) a first protein comprising or consisting of an RNA-guided DNA nuclease, as described above, and
(ii) a second protein comprising or consisting of a transposase, said transposase being a modified hyperactive PiggyBac comprising one or more amino acid mutations as compared to hyperactive PiggyBac of SEQ ID NO: 9, as described above.

In one embodiment, the fusion protein comprises or consists of:
(i) a first protein comprising or consisting of a Cas9 protein or a variant thereof, as described above, and
(ii) a second protein comprising or consisting of a transposase, said transposase being a modified hyperactive PiggyBac comprising one or more amino acid mutations as compared to hyperactive PiggyBac of SEQ ID NO: 9, as described above.

In one embodiment, the first protein and the second protein can be oriented in the fusion protein in either order.

In one embodiment, the fusion protein comprises or consists of the first protein fused at the C-terminal end of the second protein, either directly or indirectly via a linker. In other words, the fusion protein comprises or consists of, from N- to C-terminal: (i) the second protein (i.e., the transposase); (ii) optionally, a linker; and (iii) the first protein (i.e., the site-specific DNA binding protein, preferably the RNA-guided DNA nuclease; more preferably the Cas9 protein or variant thereof).

In one embodiment, the fusion protein comprises or consists of the first protein fused at the N-terminal end of the second protein, either directly or indirectly via a linker. In other words, the fusion protein comprises or consists of, from N- to C-terminal: (i) the first protein (i.e., the site-specific DNA binding protein, preferably the RNA-guided DNA nuclease; more preferably the Cas9 protein or variant thereof); (ii) optionally, a linker; and (iii) the second protein (i.e., the transposase).

In one embodiment, the fusion protein comprises a linker.

Suitable examples of linkers include peptidic linkers, between the first protein and the second protein (in any order).

In one embodiment, the peptidic linker is selected from the group comprising or consisting of (GGS)ₙ, (GGGGS)ₙ with SEQ ID NO: 133, (G)ₙ, (EAAAK)ₙ with SEQ ID NO: 134, XTEN linkers, and (XP)ₙ motif, and combinations of any of any of these, wherein n is independently an integer between 1 and 50.

In one embodiment, the linker is 12- to 24-amino acid long, or is encoded by a nucleic acid sequence that is 36- to 72-nucleotide long.

In one embodiment, the linker is a XTEN linker or a (GGS)ₙlinker.

In one embodiment, the linker is selected among the linkers shown in Table 1.

TABLE 1
Linkers

| | Nucleic acid sequence | Amino acid sequence |
|---|---|---|
| Linker | (SEQ ID NO) | (SEQ ID NO) |
| GGSx3 | ggtggatctggcggtggatctggtggcggt | GGSGGGSGGG |
| | (SEQ ID NO: 48) | (SEQ ID NO: 49) |
| GGS4x | ggagggagtggtgggtccggtggtagtggcggatcc | GGSGGSGGSGGS |
| | (SEQ ID NO: 50) | (SEQ ID NO: 51) |
| GGS5x | ggaggctccggtgggtctggtgggagcggtggtagtgg | GGSGGSGGSGGSGGS |
| | cggatcc | (SEQ ID NO: 53) |
| | (SEQ ID NO: 52) | |
| GGS6x | ggaggcagtggtgggagcggtggttccgggggtagtg | GGSGGSGGSGGSGGSGGS |
| | gtggttccgggggatcc | (SEQ ID NO: 55) |
| | (SEQ ID NO: 54) | |
| GGS7x | ggaggttctggaggctccggtgggtccgggggaagtg | GGSGGSGGSGGSGGSGGS |
| | gggggtcaggcggatcaggaggatcc | GGS |
| | (SEQ ID NO: 56) | (SEQ ID NO: 57) |
| GGS8x | ggaggtagcggaggttccggagggagcggcgggagt | GGSGGSGGSGGSGGSGGS |
| | gggggaagcgggggaagtggaggatccgggggagg | GGS |
| | atcc | (SEQ ID NO: 59) |
| | (SEQ ID NO: 58) | |
| Linker | tccggtagcgaaacaccggggacttcagaatcggccac | SGSETPGTSESATPES |
| XTEN | cccggagtct | (SEQ ID NO: 61) |
| | (SEQ ID NO: 60) | |
| Linker B | ggaagcgccggtagtgcggctgggtctggcgagttc | GSAGSAAGSGEF |
| | (SEQ ID NO: 62) | (SEQ ID NO: 63) |

In one embodiment, the linker comprises an amino acid sequence selected from the group comprising or consisting of SEQ ID NO: 49, SEQ ID NO: 51, SEQ ID NO: 53, SEQ ID NO: 55. SEQ ID NO: 57. SEQ ID NO: 59. SEQ ID NO: 61. SEQ ID NO: 63, or any combination thereof; respectively encoded by the exemplary nucleic acid sequence of SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62.

In one embodiment, the linker comprises or consists of the amino acid sequence of SEQ ID NO: 49; encoded by the exemplary nucleic acid sequence of SEQ ID NO: 48.

Also provided herein are fusion proteins obtained from the expression of any of the nucleic acid constructs provided in this disclosure.

In one embodiment, the fusion protein is a triple fusion protein.

Such triple fusion protein can comprise or consist of:
one first protein (i.e., one site-specific DNA binding protein) and two second protein (i.e., two transposases); or
two first protein (i.e., two site-specific DNA binding proteins) and one second protein (i.e., one transposase).

In one embodiment, the triple fusion comprises or consists of one first protein (i.e., one site-specific DNA binding protein) and two second protein (i.e., two transposases), and the triple fusion comprises from N- to C-terminal:
(i) the site-specific DNA binding protein, (ii) a first transposase; (iii) a second transposase; or
(i) a first transposase; (ii) the site-specific DNA binding protein, (iii) a second transposase; or
(i) a first transposase; (ii) a second transposase, (iii) the site-specific DNA binding protein.

In one embodiment, the first and second transposases are identical. In one embodiment, the first and second transposases are different. For example, the first transposase can be a hyperactive PiggyBac transposase and the second transposase can be a modified hyperactive PiggyBac transposase, chosen among any of the modified hyperactive PiggyBac transposases described herein. Alternatively, both the first and second transposases can be modified hyperactive PiggyBac transposases, but each bearing a different substitution or different combination of substitutions as described herein.

In one embodiment, the first and second transposases are capable of forming a functional dimer.

In one embodiment, the triple fusion comprises or consists of two first protein (i.e., two site-specific DNA binding proteins) and one second protein (i.e., one transposase), and the triple fusion comprises from N- to C-terminal:
(i) a first site-specific DNA binding protein, (ii) a second site-specific DNA binding protein; (iii) the transposase; or
(i) a first site-specific DNA binding protein; (ii) the transposase, (iii) a second site-specific DNA binding protein; or
(i) the transposase; (ii) a first site-specific DNA binding protein, (iii) a second site-specific DNA binding protein.

In one embodiment, the first and second site-specific DNA binding proteins are identical. In one embodiment, the first and second site-specific DNA binding proteins are different. For example, the first site-specific DNA binding protein can be a Cas9 protein and the second site-specific DNA binding protein can be a variant of a Cas9 protein, chosen among any of the Cas9 protein variants described herein. Alternatively, both the first and second site-specific DNA binding proteins can be Cas9 protein variants, but each being a different variant.

In one embodiment, the triple fusion protein optionally comprises a linker between two of its proteins or between the three proteins.

Also disclosed herein is a fusion protein comprising:
(i) the second protein comprising or consisting of a transposase; or a nucleic acid construct encoding said second protein, as described above, and
(ii) an RNA-binding protein capable of binding to at least one specific RNA sequence; or a nucleic acid construct encoding said RNA-binding protein.

In one embodiment, the fusion protein comprises a linker, as described above.

In one embodiment, the second protein comprises or consists of a transposase, said transposase being a hyperactive PiggyBac with SEQ ID NO: 9. In one embodiment, the second protein comprises or consists of a transposase, said transposase being a modified hyperactive PiggyBac comprising one or more amino acid mutations as compared to the hyperactive PiggyBac with SEQ ID NO: 9. In particular, the modified hyperactive PiggyBac can be any of those disclosed herein.

In one embodiment, the transposase/RNA-binding protein fusion can be further fused to the first protein comprising or consisting of the site-specific DNA binding protein, as described above.

In some embodiments, the RNA-binding protein is a MS2 bacteriophage coat protein (MCP) or a fragment thereof.

In some embodiments, the MCP has at least 75%, 80%, 85%, 90%, 95%, 96%, 97%, 98%, 99% or 100% identity with SEQ ID NO: 151 (encoded, e.g., by the nucleic acid sequence with SEQ ID NO: 150).

In some embodiments, the RNA-binding protein is capable of binding to at least one specific RNA sequence, said RNA sequence comprising a tetraloop. The term “tetraloop” is used interchangeably with the terms “stem loop” and “hairpin loop”.

In some embodiments, the at least one tetraloop is a MS2 RNA tetraloop-binding sequence.

In some embodiments, the tetraloop is comprised within a guide RNA (gRNA). In certain embodiments, the gRNA is in a complex with a Cas9 protein, as described above.

In some embodiments, the gRNA comprises at least one MS2 RNA tetraloop-binding sequence. In some embodiments, the gRNA comprises more than one MS2 RNA tetraloop-binding sequences.

In some embodiments, the gRNA comprising the at least one MS2 RNA tetraloop-binding sequence has at least 75%, 80%, 85%, 90%, 95%, 96%, 97%, 98%, 99% or 100% identity with SEQ ID NO: 153 (encoded, e.g., by the DNA sequence with SEQ ID NO: 152).

In some embodiments, the MCP in the fusion protein binds non-covalently to at least one MS2 RNA tetraloop-binding sequence comprised in a gRNA itself non-covalently bound to a Cas9 protein; in particular, the binding of the fusion protein to the Cas9/gRNA complex directs the excision activity of the modified hyperactive PiggyBac transposase towards the site specifically recognized by the Cas9/gRNA complex.

As will be further detailed below, certain aspects of the disclosure are also directed to vectors or plasmids (e.g., expression vectors, packaging vectors, etc.) comprising a nucleic acid construct encoding the fusion protein described herein; said vectors or plasmids being preferably suitable for expression in a host cell, e.g., mammalian cells, yeast cells, insect cells, plant cells, fungal cells, or algal cells.

According to the invention, the composition can comprise the first protein and/or the second protein (or the fusion protein comprising both), either as proteins, as described above; or as nucleic acid constructs encoding these proteins.

Targeted editing of nucleic acid sequences, e.g., the introduction of a specific modification (e.g., insertion of an exogenous nucleic acid) into genomic DNA, is a promising approach for treating human genetic diseases. To this end, the inventors aimed to provide improved nucleic acid constructs for use in genomic editing that are highly efficient at installing a desired modification; minimal off-target activity; and the ability to be programmed to edit precisely a site within the human genome.

Certain aspects of the present application are thus directed to a nucleic acid construct for use in improving site-specific insertion of an exogenous nucleic acid, e.g., a gene of interest (GOI), into a genome. In some embodiments, the GOI is a therapeutic gene, e.g., a gene that encodes a therapeutic protein. Examples of a therapeutic genes of interest include CFTR gene (Cystic fibrosis transmembrane conductance regulator) to treat Cystic Fibrosis disease; SMN1 gene (Survival motor neuron 1) to treat Spinal muscular atrophy (SMA); LRP5 gene (LDL receptor related protein 5) variant G171V to prevent osteoporosis and bone fractures; and APP gene (amyloid beta precursor protein) variant A673T to reduce Alzheimer's predisposition.

In some embodiments, the exogenous nucleic acid for insertion (e.g., the GOI) can be up to about 10 kb, up to about 15 kb, up to about 20 kb in length, up to about 25 kb in length, up to about 30 kb in length, up to about 35 kb in length, or up to about 40 kb in length.

In some embodiments, the exogenous nucleic acid for insertion can be up to 10 kb, up to 15 kb, up to 20 kb in length, up to 25 kb in length, up to 30 kb in length, up to 35 kb in length, or up to 40 kb in length, e.g., about 1 kb to about 40 kb, about 1 kb to about 39 kb, about 1 to about 38 kb, about 1 kb to about 37 kb, about 1 kb to about 36 kb, or about 1 kb to about 35 kb, for example and more preferably between 5 and 25 kb, typically between 8 and 20 kb.

In one embodiment, the composition of the invention comprises or consists of:
a. a nucleic acid construct encoding the first protein described above, comprising or consisting of a site-specific DNA binding protein described above;
b. a nucleic acid construct encoding a second protein, comprising or consisting of a transposase being a modified hyperactive PiggyBac, comprising one or more amino acid mutations as compared to hyperactive PiggyBac of SEQ ID NO: 9, as described above.

In another embodiment, the composition of the invention comprises or consists of a nucleic acid construct encoding the fusion protein described above, comprising or consisting of (i) a first protein comprising or consisting of a site-specific DNA binding protein, and (ii) a second protein comprising or consisting of a transposase being a modified hyperactive PiggyBac, comprising one or more amino acid mutations as compared to hyperactive PiggyBac of SEQ ID NO: 9, as described above.

In one embodiment, the nucleic acid construct encoding the fusion protein further comprises a nucleic acid sequence encoding a linker between the first and the second protein, as described above; or in the case of a triple fusion protein, between two of its proteins or between the three proteins.

According to the disclosure, the first and second proteins, or the fusion protein comprising or consisting of said first and second proteins, enable and/or promote site-specific insertion of an exogenous nucleic acid.

Some embodiments are directed to a plasmid or a vector (such as, e.g., an expression vector) comprising either:
a nucleic acid construct encoding the first protein; or
a nucleic acid construct encoding the second protein; or
a nucleic acid construct encoding the first protein and a nucleic acid construct encoding the second protein; or
a nucleic acid construct encoding the fusion protein or triple fusion protein.

In some embodiments, the plasmid is a packaging plasmid. In some embodiments, the plasmid further comprises a polynucleotide encoding capsid proteins, e.g., gag and pol. In some embodiments, the plasmid is combined with a second plasmid comprising a polynucleotide that encode proteins for a viral envelope (envelope plasmid); and a third plasmid comprising a nucleic acid construct comprising the exogenous nucleic acid transgene, wherein when the combination is introduced into a production cell line (e.g., eukaryotic cells, prokaryotic cells and/or cell lines), a virus particle comprising the nucleic acid constructs encoding the exogenous nucleic acid transgene and the nucleic acid construct encoding either of the first protein, second protein, both first and second proteins or fusion protein, is produced.

In some embodiments, the plasmid is combined with a second plasmid comprising a polynucleotide encoding capsid proteins, e.g., gag and pol (a packaging plasmid, wherein the packaging plasmid lacks a functional integrase), a third plasmid comprising a polynucleotide that encodes proteins for a viral envelope (envelope plasmid) and a fourth plasmid comprising a nucleic acid construct comprising the exogenous nucleic acid transgene, wherein when the combination is introduced into a production cell line (e.g., eukaryotic and prokaryotic cells and/or cell lines), a virus particle comprising the nucleic acid constructs comprising the exogenous nucleic acid transgene and the nucleic acid construct encoding either of the first protein, second protein, both first and second proteins or the fusion protein, is produced.

In one embodiment, the first protein, second protein, both first and second proteins or fusion protein, and/or the exogenous nucleic acid transgene, are delivered to a cell using a lentivirus particle.

In one embodiment, the nucleic acid construct comprises a first polynucleotide sequence encoding the first protein comprising or consisting of site-specific DNA binding protein engineered to bind a target nucleic acid sequence, a second polynucleotide sequence encoding the second protein comprising or consisting of a transposase that enables insertion of the exogenous nucleic acid transgene into the genome, and optionally, a third polynucleotide sequence comprising a nucleic acid sequence encoding a linker between the first and second polynucleotides. In some embodiments, the first protein is a zinc finger protein or a Cas9 protein or variant thereof, as described above; and/or the second protein is a modified hyperactive PiggyBac transposase, as described above.

Examples of suitable linkers to produce a fusion protein have been described hereabove.

In some embodiments, a linker is not needed because the first protein is expressed from a separate plasmid from the second protein.

In one embodiment, instead of using a linker, the first and/or the second polynucleotide sequences comprise nucleic acids encoding the first and second protein, respectively, and further comprise additional nucleotides in at least one of their ends that make the function of linker.

In one embodiment, the nucleic acid construct is in DNA or RNA form.

Also provided herein, are vectors comprising any of the nucleic acid constructs provided in this disclosure. Particularly, the vectors are suitable for expression in mammalian cells, yeast cells, insect cells, plant cells, fungal cells, or algal cells. Also provided herein, are host cells comprising any of the nucleic acid constructs or vectors provided in this disclosure.

In some embodiments, the nucleic acid construct of the disclosure is expressed in a host cell. Suitable host cells include but not limited to eukaryotic and prokaryotic cells and/or cell lines. Non-limiting examples of such host cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NS0, SP2/0-Ag14, HeLa, HEK293 (e.g., HEK293-F, HEK293-H, HEK293-T), and perC6 cells as well as insect cells such as Spodoptera fugiperda (Sf), or fungal cells such as Saccharomyces, Pichia and Schizosaccharomyces.

In some embodiments, the host cell is from a microorganism. Microorganisms which are useful for certain methods disclosed herein include, for example, bacteria (e.g., E. coli), yeast (e.g., Saccharomyces cerevisiae), and plants. The host cell can be prokaryotic or eukaryotic. In some embodiments, the host cell is eukaryotic. Suitable eukaryotic host cells include, but are not limited to, yeast cells, insect cells, plant cells, fungal cells, and algal cells.

In some embodiments, the host cell is a competent host cell. In some embodiments, the host cell is naturally competent. In some embodiments, the host cells are made competent, e.g., by a process that uses calcium chloride and heat shock. The cells used can be any cell competent, particularly eukaryotic cells, in particular mammalian, e.g. human or animal. They can be somatic or embryonic stem or differentiated. In some aspects, the cells include 293T cells, fibroblast cells, hepatocytes, muscle cells (skeletal, cardiac, smooth, blood vessel, etc.), nerve cells (neurons, glial cells, astrocytes) of epithelial cells, renal, ocular etc. It may also include, insect, plant cells, yeast, or prokaryotic cells. Additionally, primary cells may be isolated and used ex vivo for reintroduction into the subject to be treated following treatment with the nucleases (e.g. ZFNs or TALENs) or nuclease systems (e.g. CRISPR/Cas). Suitable primary cells include peripheral blood mononuclear cells (PBMC), and other blood cell subsets such as, but not limited to, T-lymphocytes such as CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In some embodiments, the host cell is transfected with a plasmid comprising a nucleic acid construct disclosed herein. In some embodiments, the plasmid comprising the nucleic acid construct is a packaging plasmid. In some embodiments, the plasmid comprising the nucleic acid construct further comprises a polynucleotide encoding capsid proteins, e.g., gag and pol. In some embodiments, the host cell is transfected with (i) the plasmid comprising the nucleic acid construct is combined in the host cell with (ii) a plasmid comprising a polynucleotide that encode proteins for a viral envelope (envelope plasmid); and (iii) a plasmid comprising an exogenous nucleic acid sequence (e.g., a GOI), wherein a virus particle comprising the exogenous nucleic acid, e.g., GOI, and the first and second proteins (either separately or as part of the fusion protein described above), is produced.

In some embodiments, the host cell is transfected with (i) the plasmid comprising the nucleic acid construct is combined with (ii) a plasmid comprising the nucleic acid construct further comprises a polynucleotide encoding capsid proteins, e.g., gag and pol (a packaging plasmid, wherein the packaging plasmid lacks a functional integrase); (iii) a plasmid comprising a polynucleotide that encode proteins for a viral envelope (envelope plasmid) and (iv) a plasmid comprising an exogenous nucleic acid sequence (e.g., a GOI), wherein a virus particle comprising the exogenous nucleic acid, e.g., GOI, and the first and second proteins (either separately or as part of the fusion protein described above), is produced.

In further embodiments, a vector, e.g., a lentiviral vector according to the disclosure, can be used for delivering the first and second proteins (either separately or as part of the fusion protein described above) encoded by a nucleic acid construct of the disclosure and an exogenous nucleic acid to an organism, e.g., a mammal, and more particularly to a mammalian target cell of interest. The lentiviral vectors comprising the first and second proteins (either separately or as part of the fusion protein described above) are able to transduce various cell types such as, for example, liver cells (e.g. hepatocytes), muscle cells, brain cells, kidney cells, retinal cells, and hematopoietic cells. In some embodiments, the target cells of the present disclosure are “non-dividing” cells. These cells include cells such as neuronal cells that do not normally divide. However, it is not intended that the present disclosure be limited to non-dividing cells (including, but not limited to muscle cells, white blood cells, spleen cells, liver cells, eye cells, epithelial cells, etc.).

In certain embodiments, a packaged first and second proteins (either separately or as part of the fusion protein described above) is administered to an organism, e.g., for gene editing of the organism's DNA. In some embodiments, the organism is a human. In some embodiments, the organism is a non-human mammal. In some embodiments, the organism is a non-human primate. In some embodiments, the organism is a rodent. In some embodiments, the organism is a sheep, a goat, a cattle, a cat, or a dog. In some embodiments, the organism is a vertebrate, an amphibian, a reptile, a fish, an insect, a fly, or a nematode. In some embodiments, the organism is a research animal. In some embodiments, the organism is genetically engineered, e.g., a genetically engineered non-human subject. The organism may be of either sex and at any stage of development. Methods for inserting a nucleic acid, for example exogenous nucleic acid, into a genome have been described. See, e.g., Yusa et al. PNAS 4(108):1531-1536 (2011); Feng et al. Nuc. Acid Res. 4(38):1204-1216 (2009); Kettlun et al. Amer. Soc. Gene and Cell Ther. 9(19):1636-1644 (2011); Skipper et al. 20(92):1-23 (2013); Li et al. PNAS 25:E2279-E2287 (2013); Mátés et al. Nature Genetics 41(6):753-761 (2009); Mali et al. Nat. Methods 10(10):957-963; Vargas et al. J. Trans. Med. 14(288):1-15 (2016); Gersbach et al. Acc. Chem. Res. 47:2309-2318 (2014); Chandrasegaran et al. Cell Gene Ther. Ins. 3(1):33-41 (2017); Wilson et al. 649:353-363 (2010); Zhao Zhang, et al. Mol Ther Nucleic Acids. 9: 230-241 (2017); Naldini L. EMBO Mol Med. 11(3) (2019); and Naldini L, et al. Hum Gene Ther. 27(10):727-728 (2016), each of which is incorporated herein by reference.

The present disclosure provides a nucleic acid construct encoding the first and second proteins (either separately or as part of the fusion protein described above), for insertion of a nucleic acid (typically exogenous nucleic acid) into a specific site of a genome. The present invention also provides the first and second proteins (either separately or as part of the fusion protein described above), for insertion of exogenous nucleic acid into a specific site of the genome. In some embodiments the exogenous nucleic acid for insertion can be up to up to 5 kb in length, up to 10 kb in length, up to 15 kb in length, 20 kb in length, up to 25 kb in length, up to 30 kb in length, up to 35 kb in length, or up to 40 kb in length, and in particular for long nucleic acids, for example between 5 kb and 25 kb, typically between 8 kb and 20 kb.

In another embodiment, methods for site-specific nucleic acid insertion into the genome are provided.

Hence, the present disclosure relates to a method for site specific integration of an exogenous nucleic acid sequence into the genome of a cell, the method comprising delivering to the cell, a composition comprising
(i) the first and second proteins (either separately or as part of the fusion protein described above) as disclosed herein, or a nucleic acid construct as disclosed herein,
(ii) an exogenous nucleic acid to be integrated into the genome of the cell, and
(iii) a guide RNA for determining site-specific integration of said exogenous nucleic acid into the genome of the cell.
wherein binding of said the first and second proteins (either separately or as part of the fusion protein described above) to the specific genomic DNA sequence in the genome of the cell results in cleavage of the genome and site-specific integration of said exogenous nucleic acid sequence into the genome of the cell as determined by the guide RNA.

In specific embodiments of said method, said exogenous nucleic acid is a nucleic acid fragment of a size of at least 5 kb, at least 6 kb, at least 7 kb, at least 8 kb, at least 9 kb, typically comprised between 5 and 25 kb, preferably between 8 and 20 kb.

In specific embodiments of said method, said exogenous nucleic acid is a therapeutic transgene to be inserted in a genome of a subject in need thereof to correct the deficiency of a genetic disorder.

In specific embodiments of said method, said composition is delivered in vitro or ex vivo, typically in a mammalian cell, preferably a human cell, and more preferably in a human cell which have been obtained from a human subject suffering from a genetic disorder.

In specific embodiments of said method, said composition is delivered in vivo into a mammal, for example a human subject in need thereof, typically for therapeutic treatment of a genetic disorder.

In some embodiments, the methods comprise contacting a target DNA with any of the fusion proteins comprising a Cas9 and a transposase described herein. For example, in some embodiments, the method comprises contacting a DNA with a fusion protein that comprises two linked polypeptides: (i) a Cas9; and (ii) a transposase, wherein the active Cas9 binds a gRNA that hybridizes to a region of the DNA, e.g., a genomic DNA.

In some embodiments, the methods comprise contacting a target DNA with any of the fusion proteins comprising a ZFP and an integrase described herein. For example, in some embodiments, the method comprises contacting a DNA with a fusion protein that comprises two linked polypeptides: (i) ZFP; and (ii) an integrase, wherein the active ZFP hybridizes to a region of the DNA, e.g., a genomic DNA.

In some embodiments, the first and second proteins (either separately or as part of the fusion protein described above) are delivered to an organism and/or a cell comprising the target DNA, e.g., genomic DNA, using a viral vector, e.g., a lentiviral particle.

Methods for lentiviral packaging have been described. See, Grandchamp et al. 9(6):1-13 (2014); Voelkel et al. 107(17):7805-7810 (2010); Tan et al. 80(4)1939-1948; Li et al. 9(8):1-9 (2014); Mátés et al. Nature Genetics 41(6):753-761 (2009); and Robert H Kutner1, et al. NATURE PROTOCOLS 4(4):495 (2009), each of which is incorporated herein by reference.

Typically, lentiviral delivery systems use a split system with different lentiviral genes on separate plasmids being used to produce a complete virus that does not contain the genetic components needed to cause the viral disease. For example, one plasmid (an envelope plasmid) can encode the proteins for the viral envelope (env); another plasmid (a packaging plasmid) can encode capsid proteins (e.g., gag and pol) and the enzymes like reverse transcriptase and/or integrase; and a further plasmid comprising the gene of interest (GOI) flanked by long-terminal repeats (for genome integration) and a psi-sequence (which displays a signal to package the gene into the virus) (a transfer plasmid). If these plasmids are simultaneously introduced into a cell, viruses will be produced containing the gene of interest without the viral genes that are needed to cause disease.

In certain aspects of the disclosure, the lentiviral vector (or particle) of the invention is obtainable by a split system, e.g., a transcomplementation system (vector/packaging system), by transfecting in vitro a permissive cell (such as 293T cells) with a plasmid containing certain components of the lentiviral vector genome, and at least one other plasmid providing, in trans, the gag, pol and env sequences encoding the polypeptides GAG, POL and the envelope protein(s), or for a portion of these polypeptides sufficient to enable formation of retroviral particles.

As an example, host cells are transfected with a) packaging plasmid, comprising a lentiviral gag and pol sequence, b) a second plasmid (envelope expression plasmid or pseudotyping env plasmid) comprising a gene encoding an envelope protein(s) (such as VSV-G), c) a plasmid vector comprising between 5′ and 3′ LTR sequences, a psi encapsidation sequence, and a transgene, and d) a plasmid vector comprising a nucleic acid construct encoding the first and second proteins (either separately or as part of the fusion protein described above) disclosed herein. In some embodiments, the nucleic acid construct encoding the first and second proteins (either separately or as part of the fusion protein described above) disclosed herein is on the packaging plasmid instead of a separate plasmid. Nucleic acids encoding gag, pol and env cDNA can be advantageously prepared according to conventional techniques, from viral gene sequences available in the prior art and databases.

In some embodiments, a lentiviral vector comprises a nucleic acid construct as described herein. In some embodiments, a lentiviral vector comprises the first and second proteins (either separately or as part of the fusion protein described above) as described herein.

The promoters used in the plasmids can be identical or different. In some embodiments, in the plasmid transcomplementation system, the envelope plasmid and the plasmid vector, respectively, to promote the expression of gag and pol of the coat protein, the mRNA of the vector genome and the transgene are promoters which can be identical or different. Such promoters can be chosen advantageously from ubiquitous promoters or specific, for example, from viral promoters CMV, TK, RSV LTR promoter and the RNA polymerase III promoter such as U6 or H1 or promoters of helper viruses encoding env, gag and pol (i.e. adenoviral, baculoviral, herpes viruses).

For the production of the lentiviral vector of the disclosure, the plasmids described herein can be introduced into host cells and the viruses are produced and harvested. Suitable cells include but not limited to eukaryotic and prokaryotic cells and/or cell lines. Non-limiting examples of such cells or cell lines generated from such cells include, e.g., COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NS0, SP2/0-Ag14, HeLa, HEK293 (e.g., HEK293-F, HEK293-H, HEK293-T), and perC6 cells as well as insect cells such as Spodoptera fugiperda (Sf), or fungal cells such as Saccharomyces, Pichia and Schizosaccharomyces.

Once host cells are transfected with the plasmids and a lentiviral vector (or particles) of the disclosure is produced, the lentiviral vectors (or particles) of the disclosure can be purified from the supernatant of the cells. Purification of the lentiviral vector to enhance the concentration can be accomplished by any suitable method, such as by density gradient purification (e.g., cesium chloride (CsCl)), by chromatography techniques (e.g., column or batch chromatography), or by ultracentrifugation. For example, the vector of the invention can be subjected to two or three CsCl density gradient purification steps. The vector, is desirably purified from infected cells using a method that comprises lysing cells, applying the lysate to a chromatography resin, eluting the virus from the chromatography resin, and collecting a fraction containing the lentiviral vector of the disclosure.

Methods of delivery of lentiviral vectors have been described. See, e.g., Vargas et al. J. Trans. Med. 14(288):1-15 (2016); Mali et al. Nat. Methods 10(10):957-963; Mátés et al. Nature Genetics 41(6):753-761 (2009); Skipper et al. 20(92):1-23 (2013).

Lentiviral vectors comprising the first and second proteins (either separately or as part of the fusion protein described above) or a nucleic acid construct coding therefor can be administered to a subject by any route. In some embodiments, a lentiviral vector of the disclosure can be delivered to cells of a subject either in vivo or ex vivo.

In some embodiments, the lentiviral vector of the disclosure can be delivered in vivo. In some embodiments, a lentiviral vector comprising the first and second proteins (either separately or as part of the fusion protein described above) encoded by a nucleic acid construct of the disclosure can be used to deliver a gene of interest and/or to target a genetic defect in a subject's DNA. In some embodiments, the lentiviral vector is administered to the subject parenterally, preferably intravascularly (including intravenously). When administered parenterally, it is preferred that the vectors be given in a pharmaceutical vehicle suitable for injection such as a sterile aqueous solution or dispersion.

In some embodiments, the lentiviral vector of the disclosure can be used ex vivo.

In some embodiments, a lentiviral vector comprising the first and second proteins (either separately or as part of the fusion protein described above) encoded by a nucleic acid construct of the disclosure can be used to deliver a gene of interest and/or target a genetic defect in a subject's DNA. In some embodiments, cells are removed from a subject and lentiviral vector comprising the first and second proteins (either separately or as part of the fusion protein described above) encoded by a nucleic acid construct of the disclosure is administered to the cells ex vivo to modify the DNA of the cells. The cells carrying the modified DNA are then expanded and reinfused back into the subject. In certain embodiments, a lentiviral vector comprising the first and second proteins (either separately or as part of the fusion protein described above) encoded by a nucleic acid construct of the disclosure can be used for Chimeric Antigen Receptor (CAR) T-cell therapy to genetically modify a patient's autologous T-cells to express a CAR specific for a tumor antigen. In a further embodiment, the modified CAR-T cells are expanded ex vivo and re-infusion back to the patient. In some embodiments, the altered T cells more specifically target cancer cells. Unlike antibody therapies, CAR-T cells are able to replicate in vivo resulting in long-term persistence.

Following administration of a lentiviral vector of the disclosure or cells modified ex vivo using a lentiviral vector of the disclosure, the subject can be monitored to detect the expression of the transgene. Dose and duration of treatment is determined individually depending on the condition or disease to be treated. A variety of conditions or diseases can be treated based on the gene expression produced by administration of the gene of interest in the vector of the present invention. The dosage of vector delivered using the method of the invention will vary depending on the desired response by the host and the vector used.

In some gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest.

Certain aspects of the disclosure are directed to a method of inserting an exogenous nucleic acid sequence into genomic DNA of an organism, comprising: identifying the specific genomic DNA sequence in the genome of the organism; administering a lentiviral particle comprising the nucleic acid construct of the disclosure to the organism to bind to the specific genomic DNA sequence and insert the exogenous nucleic acid into the genomic DNA; wherein the exogenous nucleic acid becomes integrated at the specific genomic DNA sequence.

Certain aspects of the disclosure are directed to a method for controlled, site-specific integration of a single copy or multiple copies of an exogenous nucleic acid sequence into a cell, the method comprising: a) delivering the nucleic acid construct, the vector, or the first and second proteins (either separately or as part of the fusion protein described above) of the disclosure to the cell, and b) delivering the exogenous nucleic acid to the cell; wherein binding of the first and second proteins (either separately or as part of the fusion protein described above) to the specific genomic DNA sequence in the genome of the cell, results in cleavage of the genome and integration of one or more copies of the exogenous nucleic acid into the genome of the cell. In some aspects, the delivery to the cell is by means of a lentiviral particle.

Several strategies can be used to test for integrations sites, and to screen for the best machinery for directed integration.

For analysis of the modified transposons disclosed herein, a reporter cell line with a promoter, half of the coding sequence of the GFP and a splice site donor downstream of the targeted insertion site in the genome can be used. For example, the lentiviral payload can have a fusion integrase variant followed by the inverted splice site acceptor and the other half of the GPF. The expression of GFP will occur when direct insertion happens and splicing of the GFP containing mRNA generated from the insertion site and integrated payload originates the full GFP CDS.

VPR transcomplementation systems can also be used for screening and comparing integration mutants. The transcomplementation system can be used for targeted insertion of the lentiviral payload containing a fusion integrase variant that, when expressed and loaded in the particle promote its own integration will be loaded in the viral particle using a VPR fusion. This will complement in trans the integration defective IN coded in the packaging vector used for particle production. Other methods that can be used for integration mapping including IC, or FISH probes. Targeted insertion can also be screened by TCRa or RFP targeted disruption, or GFP activation by targeted splice site integration.

For the FISH approach to co-staining of the insertion and target region in the chromatin, a Fluorescence in situ hybridization to localize the gene of interest transposon in the Hek293T genome can be performed. Hek293T can be transfected with 1) GOI-transposon 2) Programmable transposase and 3) gRNA to PPP1R12. Probes are designed to target the PPP1R12 gene, CD46 gene (as negative control) and GOI, and can be synthesized with Nick Translation Mix (Sigma) from PCR amplified DNA.

In some embodiments, the first and second proteins (either separately or as part of the fusion protein described above) comprising a modified transposase as disclosed herein improve the specificity of insertion of the exogenous nucleic acid into the genome compared to a wild-type transposase (or a fusion protein containing the corresponding wildtype transposase), e.g., as determined by a Genetrap assay. In some embodiments, HEK293T cells, or any other permissible cells, are transfected or transduced with lentiviral particles with the following plasmids or payloads: (i) a plasmid comprising a gRNA that targets a specific region of DNA, (ii) a plasmid comprising the nucleic acid construct of the disclosure encoding the first and second proteins (either separately or as part of the fusion protein described above) with the second protein being a modified transposase, and (iii) a genetrap plasmid comprising a nucleic acid sequence encoding a reporter protein, e.g., GFP, that lacks a promoter. In some embodiments, the genetrap plasmid further comprises a transposon with inverted repeats.

In some embodiments, the percent of cells containing the GFP insertion can be determined by flow cytometry. In some embodiments, the first and second proteins (either separately or as part of the fusion protein described above) with the second protein being a modified transposase increase the percent of cells containing insertion of GFP by at least 5%, at least 10%, at least 15%, at least 20%, at least 25%, or at least 30% compared to the corresponding wildtype protein. In some embodiments, the first and second proteins (either separately or as part of the fusion protein described above) with the second protein being a modified transposase increase the percent of cells containing insertion of GFP by about 15-30%.

In some embodiments, the percent of insertions at the targeted site and percent of coverage at the target site (number of reads per insertion site) can be determined by genomic DNA extraction and targeted sequencing with oligonucleotides specific for viral LTRs. In some embodiments, the first and second proteins (either separately or as part of the fusion protein described above) with the second protein being a modified transposase increase the percent of insertions at the targeted site by at least 10-fold, at least 20-fold, at least 30-fold, at least 40-fold, at least 50-fold, at least 60-fold, at least 70-fold, at least 80-fold, at least 90-fold, or at least 100-fold compared to the corresponding wildtype protein. In some embodiments, the percent of insertions at the targeted site is increased by about 10-100 fold. In some embodiments, the first and second proteins (either separately or as part of the fusion protein described above) with the second protein being a modified transposase increase the percent of coverage at the target site (number of reads per insertion site) by at least 10-fold, at least 20-fold, at least 30-fold, at least 40-fold, at least 50-fold, at least 60-fold, at least 70-fold, at least 80-fold, at least 90-fold, at least 100-fold, at least 110-fold, at least 120-fold, at least 130-fold, at least 140-fold, at least 150-fold, at least 160-fold, at least 170-fold, at least 180-fold, at least 190-fold, or at least 200-fold compared to the corresponding wildtype protein. In some embodiments, the percent of coverage at the target site (number of reads per insertion site) by at least 100-fold.

In some embodiments, the percent of insertions at the targeted site and percent of coverage at the target site (number of reads per insertion site) can be determined by genomic DNA extraction and targeted sequencing with oligonucleotides specific for viral inserted LTR.

Possible applications of lentiviral vectors comprising the first and second proteins (either separately or as part of the fusion protein described above) of the disclosure include gene therapy, i.e., the gene transfer in any mammal cell, in particular in human cells. It may be dividing cells or quiescent cells, cells belonging to the central organs or peripheral organs such as the liver, pancreas, muscle, heart, etc. Gene therapy may allow the expression of proteins, e.g. neurotrophic factors, enzymes, transcription factors, receptors, etc. Lentiviral vectors according to the invention may also particularly suitable for research purposes.

In some embodiments, a nucleic acid constructs, the first and second proteins (either separately or as part of the fusion protein described above), and/or a lentiviral vector of the disclosure is administered to a subject to treat a disease. In some embodiments, the disease is a genetic disorder that can benefit from gene therapy.

In some embodiments, the first and second proteins (either separately or as part of the fusion protein described above), or the nucleic acid constructs coding therefor, or the kit or composition as disclosed hereafter or the lentiviral vectors comprising the first and second proteins (either separately or as part of the fusion protein described above) or nucleic acid constructs according to the disclosure can be used as a medicament.

The lentiviral vector according to the disclosure may be particularly suitable for treating a genetic disease in a subject.

The present invention also relates to a composition comprising
(i) a RNA guided nuclease or zinc finger nuclease,
(ii) a transposase,
(iii) a guide RNA, and
(iv) a nucleic acid or gene of interest, for example an exogenous nucleic acid for insertion in a genome,
wherein said transposase is a modified hyperactive Piggybac, comprising one or more amino acid mutations as compared to hyperactive Piggybac of SEQ ID NO:9.

In one embodiment, the modified hyperactive PiggyBac mutation comprises the amino acid substitution R372A/K375A/D450N.

In a preferred embodiment, the modified hyperactive PiggyBac mutation does not comprise the amino acid substitution R372A/K375A/D450N.

In some embodiments, the modified hyperactive PiggyBac mutation comprises the following amino acid substitution or combination of amino acid substitution of S351A/R372A/K375A/R388A/D450N/W465A/S573A/M589V/S592G/F594L, R245A/R275A/R277A/R372A/W465A/M589V, R275A/325A/R372A/T560A, N347A/D450N, N347S/D450N/T560A/S573A/F594L, R202K/R275A/N347S/R372A/D450N/T560A/F594L, R275A/N347S/K375A/D450N/S592G, R275A/N347S/R372A/D450N/T560A/F594L, R275A/R277A/N347S/R372A/D450N/T560A/S564P/F594L, R245A/N347S/R372A/D450N/T560A/S564P/S573A/S592G, R277A/G325A/N347A/K375A/D450N/T560A/S564P/S573A/S592G/F594L, V34M/R275A/G325A/N347S/S351A/R372A/K375A/D450N/T560A/S564P, G325A/N347S/K375A/D450N/S573A/M589V/S592G, S230N/R277A/N347S/K375A/D450N, T43I/R372A/K375A/A411T/D450N, G325A/N347S/S351A/K375A/D450N/S573A/M589V/S592G, Y177H/R275A/G325A/K375A/D450N/T560A/S564P/S592G, the position number corresponding to the amino acid number of the hyperactive PiggyBac sequence (SEQ ID NO: 9).

In a preferred embodiment, the modified hyperactive PiggyBac mutation comprises the following amino acid substitution or combination of amino acid substitution of R245A/R275A/R277A/R372A/W465A/M589V, R275A/325A/R372A/T560A, N347A/D450N, N347S/D450N/T560A/S573A/F594L, R202K/R275A/N347S/R372A/D450N/T560A/F594L, R275A/N347S/K375A/D450N/S592G, R275A/N347S/R372A/D450N/T560A/F594L, R275A/R277A/N347S/R372A/D450N/T560A/S564P/F594L, R245A/N347S/R372A/D450N/T560A/S564P/S573A/S592G, R277A/G325A/N347A/K375A/D450N/T560A/S564P/S573A/S592G/F594L, G325A/N347S/K375A/D450N/S573A/M589V/S592G, S230N/R277A/N347S/K375A/D450N, G325A/N347S/S351A/K375A/D450N/S573A/M589V/S592G, Y177H/R275A/G325A/K375A/D450N/T560A/S564P/S592G, the position number corresponding to the amino acid number of the hyperactive PiggyBac sequence (SEQ ID NO: 9).

In some embodiments the RNA guided nuclease is a Cas9 protein. In some embodiments the RNA guided nuclease is a SpCas9 protein. In some embodiments the RNA guided nuclease is a SaCas9 protein.

The present invention also relates to a composition comprising nucleic acids encoding:
(i) a RNA guided nuclease or zinc finger nuclease, as described hereinabove,
(ii) a transposase, as described hereinabove,
(iii) a guide RNA, and
(iv) a nucleic acid or gene of interest, for example an exogenous nucleic acid for insertion in a genome.

In some embodiments, the nucleic acids of the composition are expressed in a cell through a suitable expression vector. As used herein, the term “expression vector” refers to a vector comprising a polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

In a preferred embodiment, the two nucleic acids are co-expressed in the same cell or cell population. In some embodiments, the two nucleic acids are co-expressed concomitantly. In another embodiment, the nucleic acid encoding an RNA guided nuclease or zinc finger nuclease is expressed first. In another embodiment, the nucleic acid encoding a transposase is expressed first.

The invention further relates to a composition comprising
(i) a fusion protein comprising a RNA-guided nuclease and a transposase as disclosed herein, or a nucleic acid encoding thereof,
(ii) a transposase as disclosed herein, or a nucleic acid encoding thereof,
(iii) a guide RNA, and
(iv) a nucleic acid or gene of interest, for example an exogenous nucleic acid for insertion in a genome.

The invention further relates to a composition comprising
(i) a first fusion protein comprising a RNA-guided nuclease and a transposase as disclosed herein, or a nucleic acid encoding thereof,
(ii) a second fusion protein comprising a RNA binding protein engineered to bind to at least one specific RNA sequence, and a transposase as disclosed herein, or a nucleic acid encoding thereof,
(iii) a guide RNA, and
(iv) a nucleic acid or gene of interest, for example an exogenous nucleic acid for insertion in a genome.

The present disclosure also provides compositions for practicing the disclosed methods as described herein. In some embodiments, a composition comprises a nucleic acid construct or a vector as defined in this disclosure, and a polynucleotide sequence encoding an exogenous nucleic acid for insertion in a genome, contained in in or bound to a packaging vector.

The present disclosure further relates to a composition comprising
(i) a fusion protein comprising a RNA-guided nuclease and a transposase as disclosed herein or a nucleic acid encoding said fusion protein,
(ii) a guide RNA, and
(iii) a nucleic acid or gene of interest, for example an exogenous nucleic acid for insertion in a genome.

In specific embodiments, said nucleic acid or gene of interest is a large DNA fragment, typically having a size between 5 kb and 25 kb, and more preferably between 8 kb and 20 kb.

Also provided by the present disclosure are kits for practicing the disclosed methods, as described herein. The kit can contain the nucleic acid constructs or fusion proteins as described herein. In some aspects, the kit can contain the lentiviral particles containing the nucleic acid constructs or fusion proteins as described herein.

The subject kit can further include instructions for using the components of the kit to practice the subject methods. The instructions for practicing the subject methods are generally recorded on a suitable recording medium. For example, the instructions can be printed on a substrate, such as paper or plastic, etc. As such, the instructions can be present in the kit as a package insert, in the labeling of the container of the kit or components thereof (i.e., associated with the packaging or subpackaging), etc. In other embodiments, the instructions are present as an electronic storage data file present on a suitable computer readable storage medium. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

The disclosure typically relates to a kit, comprising
a first composition including
(i) a first fusion protein as defined herein, or a nucleic acid encoding said first fusion protein, and wherein said first fusion protein comprises an amino acid sequence of a first guided RNA nickase Cas9, typically SpCas9 nickase of SEQ ID NO:70 fused to a modified hyperactive Piggybac, and,
(ii) a first guided RNA nucleic acid,
a second composition including
(iii) a second fusion protein as defined herein, or a nucleic acid encoding said second fusion protein, and wherein said second fusion protein comprises an amino sequence of a second guided RNA nickase Cas9, typically SaCas9 nickase of SEQ ID NO:76 fused to a modified hyperactive Piggybac,
(iv) a second guided RNA nucleic acid, optionally, an nucleic acid for insertion in a genome, for example a nucleic acid having a size between 5 kb and 25 kb, and more preferably between 8 kb and 20 kb.
   wherein said first and second fusion protein are capable of forming heterodimerization and making double cuts determined by said first and second guided RNA at adjacent sites of a genomic DNA region, and optionally inserting said nucleic acid between the adjacent sites.

In specific embodiments, the composition or kit comprises exogenous nucleic acid in a minicircle, a plasmid or a viral vector, in particular in non-integrating viral vector, for example or non-integrating lentiviral vector.

In specific embodiments, the composition or kit as disclosed herein is comprised in a nanoparticle.

In specific embodiments, said composition is a nucleic acid composition comprising
(i) a nucleic acid construct encoding the fusion protein as disclosed herein,
(ii) a guide RNA, and
(iii) a nucleic acid or gene of interest, for example an exogenous nucleic acid for insertion in a genome.

In specific embodiments, said kit comprising
a first composition including
(i) a nucleic acid construct encoding a first fusion protein as disclosed herein, and wherein said first fusion protein comprises an amino acid sequence of a first guided RNA nickase Cas9, typically SpCas9 nickase of SEQ ID NO:70 fused to a modified hyperactive Piggybac, and,
(ii) a first guided RNA nucleic acid,
a second composition including
(iii) a nucleic acid construct encoding said second fusion protein as disclosed herein, and wherein said second fusion protein comprises an amino sequence of a second guided RNA nickase Cas9, typically SaCas9 nickase of SEQ ID NO:76 fused to a modified hyperactive Piggybac, and,
(iv) a second guided RNA nucleic acid,

In specific embodiments, said kit or composition is for use as a drug, in particular in treating disorders in human, for example for treating genetic deficiencies in a human subject in need thereof.

In some embodiments, the nucleic acid construct is in form of RNA, DNA or protein, and the polynucleotide sequence encoding the exogenous nucleic acid is in form of RNA or DNA, depending on the method of delivery. Particularly, the polynucleotide sequence encoding the exogenous nucleic acid is in form of RNA.

In some embodiments, the composition or kit is viral-free and the packaging vector is a nanoparticle e.g. a polymeric or lipidic nanoparticle. The packaging vector can also be a carrier which is bound to the elements of the composition. In some embodiments, the composition is contained in a viral vector, particularly a lentiviral particle.

In some embodiments, the composition or kit comprises (a) the nucleic acid construct described herein (e.g. comprising Cas9 and a transposase) in form of RNA, (b) a guide RNA if needed (e.g. as separate lineal single strand RNA molecule), and (c) a polynucleotide comprising the exogenous gene for insertion in DNA form (e.g. in a vector), contained in in or bound to a packaging vector.

In some embodiments, the composition comprises (a) the fusion protein described herein (e.g. comprising Cas9 and a transposase) in form of protein, (b) a guide RNA if needed (e.g. as separate lineal single strand RNA molecule), wherein the fusion protein and the guide RNA form a ribonucleic protein complex (RNP), and (c) a polynucleotide comprising the exogenous gene for insertion in DNA form (e.g. in a vector), contained in in or bound to a packaging vector.

In some embodiments, the composition comprises (a) the nucleic acid construct described herein (e.g. comprising Cas9 and a transposase) in form of DNA, (b) a guide RNA if needed (e.g. as separate lineal RNA molecule or as DNA in a vector), and (c) a polynucleotide comprising the exogenous gene for insertion in DNA form (e.g. in a vector), contained in in or bound to a packaging vector.

In some embodiments, the composition comprises (a) the fusion protein described herein (e.g. comprising Cas9 and an integrase) in form of protein, (b) a guide RNA if needed (e.g. as separate RNA molecule complexing with the fusion protein), and (c) a polynucleotide comprising the exogenous gene for insertion, contained in in or bound to a packaging vector. In a particular embodiment, the packaging vector is a lentiviral particle. In some embodiments, the (a) fusion protein is bound to the lentiviral capside by means of gag-pol or VPR (Viral Protein R). In some embodiments, the (c) polynucleotide is in form of RNA as payload of the integrase.

In a particular embodiment, when ZFP is used, (b) the guide RNA can not be needed.

The invention further relates to a composition comprising
(i) a fusion protein comprising a RNA binding protein engineered to bind to at least one specific RNA sequence, a DNA binding protein enabling the insertion of an exogenous nucleic acid into the genome, and a linker connecting the first and second protein,
(ii) a Cas9 protein, and
(iii) a guide RNA comprising said at least one specific RNA sequence for fusion protein binding,
(iv) a nucleic acid or gene of interest, for example an exogenous nucleic acid for insertion in a genome
wherein the DNA binding protein is a modified transposase of this disclosure, typically a modified hyperactive Piggybac comprising one or more amino acid mutations to increase excision activity as compared to unmodified hyperactive Piggybac, and one or more amino acid mutations to decrease DNA binding activity as compared to unmodified hyperactive Piggybac according to SEQ ID NO: 9,

In some embodiments, the RNA binding protein is the MS2 bacteriophage coat protein (MCP).

In some embodiments, the at least one RNA sequence recognized by the MCP of the fusion protein is a tetraloop. As used herein, the term “tetraloop” is used interchangeably with the terms “stem loop” and “hairpin loop”. In some embodiments, the at least one RNA tetraloop is a MS2 RNA tetraloop binding sequence.

In some embodiments the guide RNA comprises at least one MS2 RNA tetraloop binding sequence. In some embodiments, the gRNA comprises more than one MS2 RNA tetraloop binding sequences. As used herein, the term “more than one” means 2, 3, 4, 5, 6, 7, 8, 9, 10, 11 or more.

Various embodiments are described in the claims. Some additional embodiments are disclosed hereafter:
E1. A fusion protein comprising
   (i) a first protein consisting of an RNA guided nuclease or zinc finger nuclease,
   (ii) a second protein consisting of a transposase, and
   (iii) optionally, a linker connecting the first and second protein,
   wherein said transposase is a modified hyperactive Piggybac, comprising one or more amino acid mutations as compared to hyperactive Piggybac of SEQ ID NO:9,
   and wherein said first protein is fused at the C-terminal end of the second protein directly, or indirectly via a linker.
E2. The fusion protein of Embodiment 1, wherein said transposase is a modified hyperactive Piggybac, comprising one or more amino acid mutations to increase excision activity as compared to unmodified hyperactive Piggybac, and one or more amino acid mutations to decrease DNA binding activity as compared to unmodified hyperactive Piggybac,
E3. The fusion protein of Embodiment 2, wherein said one or more amino acid mutations to increase excision activity are selected among the amino acid mutations within the region defined by the amino acid position numbers [194-200], [214-222], [434-442] or [446-456], for example amino acid substitution at the position D198, D201, R202, M212, or S213; said position number corresponding to the amino acid number of unmodified hyperactive Piggybac of SEQ ID NO:9.
E4. The fusion protein of any one of Embodiments 1-3, wherein said one or more amino acid mutations are selected among the amino acid substitutions which increase excision activity at position of M194 or D450, said position number corresponding to the amino acid number of unmodified hyperactive Piggybac of SEQ ID NO:9, preferably selected among the amino acid substitutions M194V and/or D450N.
E5. The fusion protein of any one of Embodiments 1 to 4, wherein said one or more amino acid mutations are selected among the amino acid substitutions which decrease DNA binding activity at position R372, K375, R376, E377, and/or E380, said position number corresponding to the amino acid number of unmodified hyperactive Piggybac of SEQ ID NO:9, preferably selected among the amino acid substitutions R372A, K375A, R376A, E377A, and/or E380A.
E6. The fusion protein of any one of Embodiments 1 to 5, wherein the modified hyperactive Piggybac includes at least one amino acid substitution to increase excision activity at position D450, and at least two amino acid substitutions to decrease DNA binding activity at position R372 and K375, preferably said modified transposase of hyperactive Piggybac includes the triple mutations D450N, R372A and K375A, said position number corresponding to the amino acid number of unmodified hyperactive Piggybac of SEQ ID NO:9.
E7. The fusion protein of any one of Embodiments 1 to 6, wherein the modified hyperactive Piggybac further comprises at least one mutation in the region defined by the amino acid position numbers [158-169], for example A166S; and/or at least one mutation at position Y527, R518, K525, N463.
E8. The fusion protein of any one of Embodiments 1 to 7, wherein said modified hyperactive Piggybac comprises an amino acid sequence having at least 85%, at least 90%, at least 95% identity, or 100% identity to the modified hyperactive Piggybac of SEQ ID NO:1.
E9. The fusion protein of any one of Embodiments 1 to 8, wherein said modified hyperactive Piggybac is a variant of the hyperactive Piggybac of SEQ ID NO:1 with one or more amino acid substitutions, typically with no more than 1, 2, 3, 4, 5, 6, 7, 8, 9 or 10 amino acid substitutions.
E10. The fusion protein of any one of Embodiments 1 to 9, wherein said modified hyperactive Piggybac further comprises one or more amino acid substitutions at the position number 245, 268, 275, 277, 287, 290, 315, 325, 341, 346, 347, 350, 351, 356, 357, 388, 409, 412, 432, 447, 460, 461, 465, 517, 560, 564, 571, 573, 576, 586, 587, 589, 592, and/or 594, the position number corresponding to the amino acid number of the hyperactive PiggyBac sequence (SEQ ID NO: 9).
E11. The fusion protein of Embodiment 10, wherein the modified hyperactive PiggyBac mutation comprises the following amino acid substitution or combination of amino acid substitution of R245A, D268N, R275A, R277A, K287A, K290A, K287A/K290A, R315A, G325A, R341A, D346N, N347A, N347S, T350A, S351E, S351P, S351A, K356E, N357A, R388A, K409A, K412A, K432A, D447A, D447N, D450N, R460A, K461A, W465A, S517A, T560A, S564P, S571N, S573A, K576A, H586A, I587A, M589V, S592G, or F594L, D450N/R372A/K375A, R275A/R277A, K409A/K412A, R460A/K461A, R275A/R277A/N347S/K375A/T560A/S573A/M589V/S592G and R245A/R275A/R277A/R372A/W465A, the position number corresponding to the amino acid number of the hyperactive PiggyBac sequence (SEQ ID NO: 9).
E12. The fusion protein of Embodiment 10, wherein the modified hyperactive PiggyBac mutation comprises the following amino acid substitution or combination of amino acid substitution of R372A/K375A/D450N, R372A/K375A/R376A/D450N, K375A/R376A/E377A/E380A/D450N, R372A/K375A/R376A/E377A/E380A/D450N, M194V, R376A, E377A, E380A M194V/R372A/K375A, S351A/R372A/K375A/R388A/D450N/W465A/S573A/M589V/S592G/F594L, R245A/R275A/R277A/R372A/W465A/M589V, R275A/325A/R372A/T560A, N347A/D450N, N347S/D450N/T560A/S573A/F594L, R202K/R275A/N347S/R372A/D450N/T560A/F594L,R275A/N347S/K375A/D4 50N/S592G, R275A/N347S/R372A/D450N/T560A/F594L, R275A/R277A/N347S/R372A/D450N/T560A/S564P/F594L; the position number corresponding to the amino acid number of the hyperactive PiggyBac sequence (SEQ ID NO: 9) typically said modified transposase has an amino acid sequence selected among any of SEQ ID NO: 1-8 and 10-18.
E13. The fusion protein of any one of Embodiments 1-12, wherein the linker is a peptidic linker which comprises a XTEN sequence or a GGS sequence, preferably a XTEN sequence.
E14. The fusion protein of any one of Embodiments 1-13, wherein the linker is a peptidic linker having between 3 to 50 amino acids in length, typically selected among any of SEQ ID NO: 49, 51, 53, 55, 57, 59, 61.
E15. The fusion protein of any one of Embodiments 1-14, wherein said first protein is a Cas9 protein comprising an active DNA cleavage domain and a guide RNA binding domain.
E16. The fusion protein of any one of Embodiments 1-15, wherein said first protein is a nuclease protein comprising an active DNA cleavage domain and a guide RNA binding domain and having at least 80%, 90%, 95%, 99% or at least 100% identity to a Streptococcus pyogenes Cas9 of SEQ ID NO:31, SaCas9 of SEQ ID NO:72, Cpf1 of SEQ ID NO:74, CjCas9 of SEQ ID NO:29, SpCas9 nickase of SEQ ID NO:70, CasX of SEQ ID NO:75, or SaCas9 nickase of SEQ ID NO:76.
E17. The fusion protein of any one of Embodiments 1-16, wherein said first protein is a Cas9 protein selected from the group consisting of a SaCas9 of SEQ ID NO:72 or Streptococcus pyogenes Cas9 of SEQ ID NO:31.
E18. The fusion protein of any one of Embodiments 1 to 17, which is a triple fusion protein comprising
   (i) a first protein consisting of an RNA guided nuclease or nickase,
   (ii) a second protein consisting of a first transposase,
   (iii) a third protein consisting of a second transposase and,
   (iv) optionally, peptidic linkers between the first and second protein and the second and the third protein,
   and wherein the first and second transposases have identical or different sequences of modified Piggybac transposase, for example as defined in any one of Embodiments 3-12.
E19. The fusion protein of any one of Embodiments 1 to 14, wherein said first protein is a zinc finger protein of SEQ ID NO:33.
E20. A composition comprising
   (i) a fusion protein of any of Embodiments 1-19, or a nucleic acid encoding said fusion protein,
   (ii) a guide RNA, and
   (iii) an exogenous nucleic acid for insertion in a genome.
E21. The composition of Embodiment 20, wherein said exogenous nucleic acid is a large DNA fragment, typically having a size between 5 kb and 25 kb, and more preferably between 8 kb and 20 kb.
E22. A kit, comprising
   (i) a first composition including
      a first fusion protein as defined in any one of Embodiments 1 and 18 or a nucleic acid encoding said first fusion protein, and wherein said first fusion protein comprises an amino acid sequence of a first guided RNA nickase Cas9, typically SpCas9 nickase of SEQ ID NO:70 fused to a modified hyperactive Piggybac,
      a first guided RNA nucleic acid,
   (ii) a second composition including
      a second fusion protein as defined in any one of Embodiments 1 to 18, or a nucleic acid encoding said second fusion protein, and wherein said second fusion protein comprises an amino sequence of a second guided RNA nickase Cas9, typically SaCas9 nickase of SEQ ID NO:76 fused to a modified hyperactive Piggybac,
      a second guided RNA nucleic acid,
   (iii) optionally, an exogenous nucleic acid for insertion in a genome, for example an exogenous nucleic acid having a size between 5 kb and 25 kb, and more preferably between 8 kb and 20 kb.
   wherein said first and second fusion proteins are capable of forming heterodimerization and making double cuts determined by said first and second guided RNA at adjacent sites of a genomic DNA region, and optionally inserting said exogenous nucleic acid between the adjacent sites.
E23. The composition or kit of Embodiments 20-22, wherein said exogenous nucleic acid is comprised in a minicircle, a plasmid or a viral vector, in particular non-integrating viral vector, for example or non-integrating lentiviral vector.
E24. The composition or kit of Embodiments 20-23, wherein the compositions are comprised in a nanoparticle.
E25. A modified hyperactive Piggybac transposase, comprising at least one amino acid mutation to increase excision activity as compared to unmodified hyperactive Piggybac, and/or at least one amino acid mutation to decrease DNA binding activity as compared to unmodified hyperactive Piggybac, wherein said at least one mutation to increase excision activity is an amino acid substitution of M at position 194, typically M194V and/or wherein at least one amino acid mutation to decrease DNA binding activity are selected among the amino acid substitutions at positions R376, E377, and E380, typically R376A, E377A, and/or E380A.
E26. A modified hyperactive Piggybac transposase, which comprises the following combination of mutations R372A/K375A/D450N, R372A/K375A/R376A/D450N, K375A/R376A/E377A/E380A/D450N, R372A/K375A/R376A/E377A/E380A/D450N, M194V, R376A, E377A, E380A, M194V/R372A/K375A, S351A/R372A/K375A/R388A/D450N/W465A/S573A/M589V/S592G/F594L, R245A/R275A/R277A/R372A/W465A/M589V, R275A/325A/R372A/T560A, N347A/D450N, N347S/D450N/T560A/S573A/F594L, R202K/R275A/N347S/R372A/D450N/T560A/F594L,R275A/N347S/K375A/D4 50N/S592G, R275A/N347S/R372A/D450N/T560A/F594L, R275A/R277A/N347S/R372A/D450N/T560A/S564P/F594L as compared to unmodified hyperactive Piggybac of SEQ ID NO:9.
E27. The modified hyperactive Piggybac transposase of Embodiment 25 or 26, further comprising one or more amino acid mutations selected from the group consisting: amino acid at position 245 is A, amino acid at position 275 is R or A, amino acid at position 277 is R or A, amino acid at position 325 is A or G, amino acid at position 347 is N or A, amino acid at position 351 is E, P or A, amino acid at position 372 is R, amino acid at position 375 is A, amino acid at position 450 is D or N, amino acid at position 465 is W or A, amino acid at position 560 is T or A, amino acid at position 564 is P or S, amino acid at position 573 is S or A, amino acid at position 592 is G or S, and amino acid at position 594 is L or F.
E28. The modified hyperactive PiggyBac transposase of Embodiment 25 or 26, which comprises 1, 2, 3, 4, 5, 6, 7, 8, 9 or 10 additional mutations as compared to unmodified hyperactive PiggyBac of SEQ ID NO: 9, wherein the modified hyperactive PiggyBac shows decreased DNA binding activity and/or increased excision activity compared to hyperactive PiggyBac of SEQ ID NO:9.
E29. A nucleic acid encoding the fusion protein of any one of Embodiments 1 to 18, typically a messenger RNA (mRNA).
E30. The nucleic acid encoding the fusion protein of Embodiment 28, which comprises a sequence selected from the group consisting of SEQ ID NO: 110-112 or their corresponding mRNA sequence.
E31. A nucleic acid encoding the modified hyperactive Piggybac of any one of Embodiments 25-28.
E32. An expression vector comprising the nucleic acid of any one of Embodiments 29-31.
E33. A host cell comprising the nucleic acid of any one of Embodiments 29-31, or the expression vector of Embodiment 32.
E34. A method for site specific integration of an exogenous nucleic acid sequence into the genome of a cell, the method comprising delivering to the cell, a composition comprising
   (i) a fusion protein of any one of Embodiments 1 to 18, or a nucleic acid of any one of Embodiments 29-31,
   (ii) an exogenous nucleic acid to be integrated into the genome of the cell, and
   (iii) a guide RNA for determining site-specific integration of said exogenous nucleic acid into the genome of the cell.
   wherein binding of said fusion protein to the specific genomic DNA sequence in the genome of the cell results in cleavage of the genome and site-specific integration of said exogenous nucleic acid sequence into the genome of the cell as determined by the guide RNA.
E35. The method of Embodiment 34, wherein said exogenous nucleic acid is a nucleic acid fragment of a size of at least 5 kb, at least 6 kb, at least 7 kb, at least 8 kb, at least 9 kb, typically comprised between 5 and 25 kb, preferably between 8 and 20 kb.
E36. The method of Embodiment 34 or 35, wherein said exogenous nucleic acid is a therapeutic transgene to be inserted in a genome of a subject in need thereof to correct the deficiency of a genetic disorder.
E37. The method of any one of Embodiments 34-36, wherein said composition is delivered in vitro or ex vivo, typically in a mammalian cell, preferably a human cell, and more preferably in a human cell which have been obtained from a human subject suffering from a genetic disorder.
E38. The method of any one of Embodiment 34-37, wherein said composition is delivered in vivo into a mammal, for example a human subject in need thereof, typically for therapeutic treatment of a genetic disorder.
E39. A method of inserting an exogenous nucleic acid sequence into genomic DNA of an organism, comprising: administering one or more compositions or kits as defined in any one of Embodiment 20-24, to the organism such that the fusion protein comprised in said one or more compositions or kits bind to a specific genomic DNA sequence and enables the insertion of the exogenous nucleic acid comprised in said composition into the genomic DNA;
   wherein the exogenous nucleic acid becomes integrated at a specific site of the genome of a cell of said organism, for example, a non-human organism or a human subject in need thereof.

### Examples

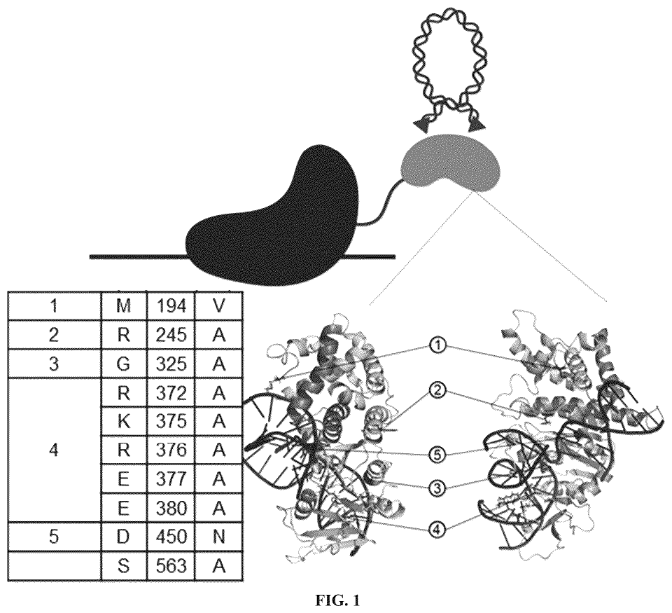

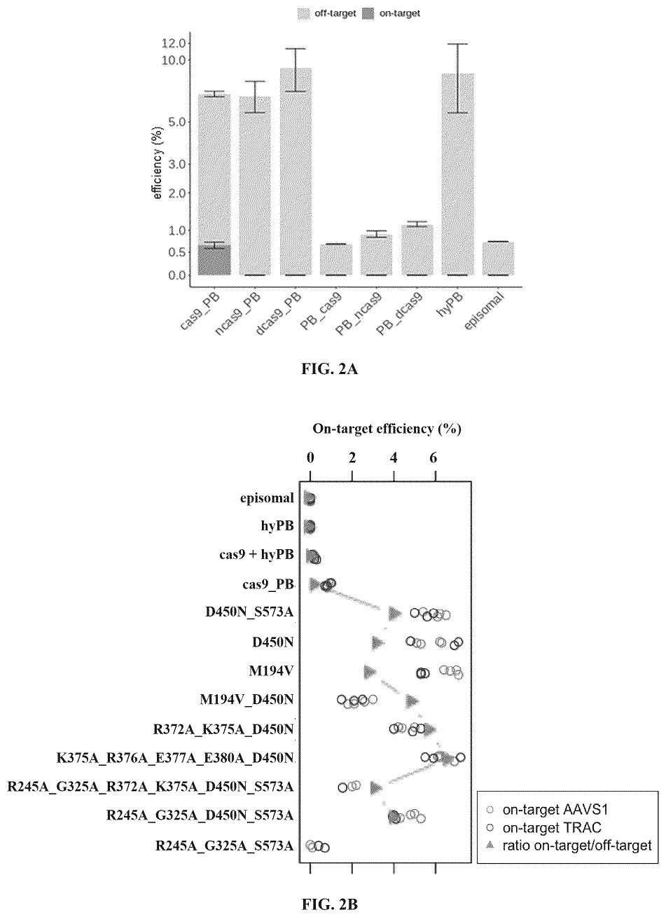

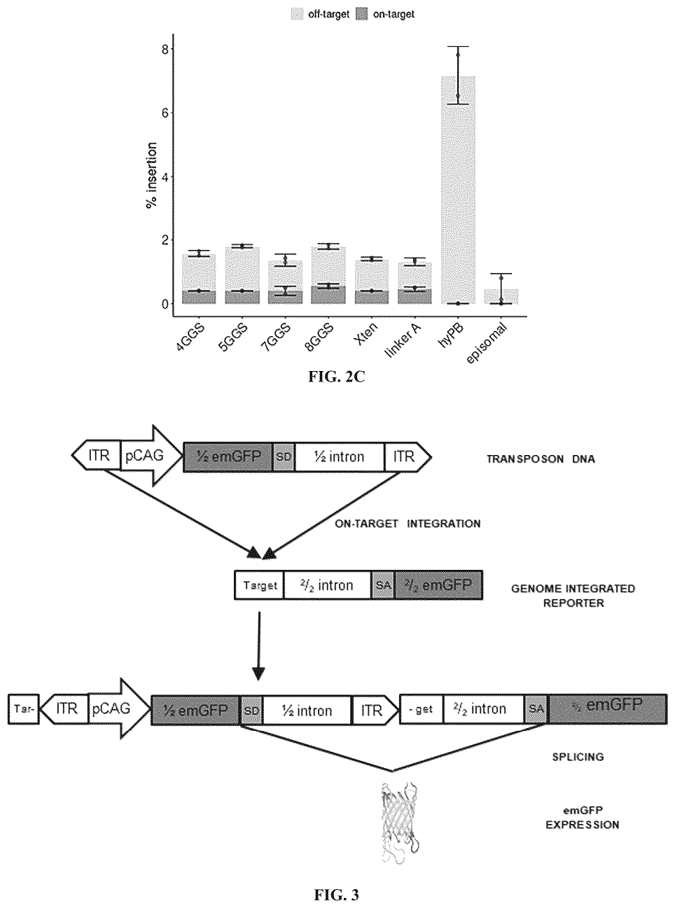

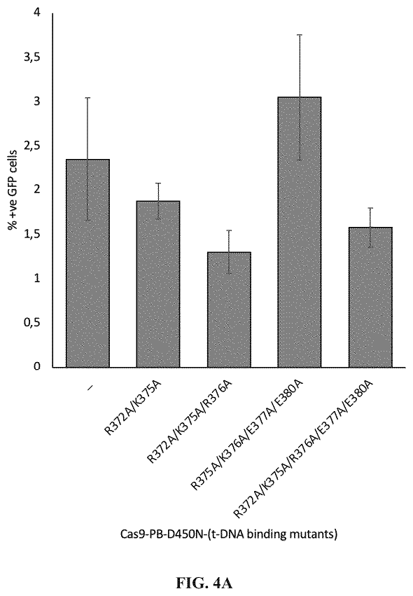

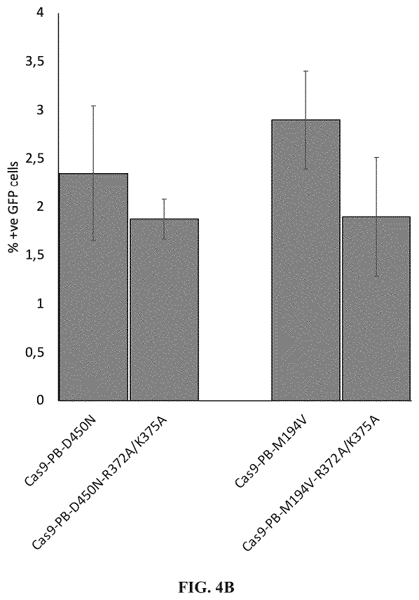

To achieve a programmable transposase, our principle of design attempted to combine the precise genome-targeting of CRISPR systems with PB variants that exhibit enhanced insertion and excision activity and lower target DNA binding activity. We started by fusing a nuclease SpCas9 with a PB transposase (FIG. 1). We constructed a diverse library of PB and SpCas9 variants (FIG. 2a, b, c) where we tested a library of PB variants, 3 SpCas9 variants (nuclease SpCas9 (cas9), dead SpCas9 (dcas9), nickase SpCas9 (ncas9)), and 6 linkers (4×GGS, 5×GGS, 7×GGS, 8×GGS, XTEN, FOKI).

Different parts of the PB transposase were diversified. Special emphasis focused on the catalytic core domain which is formed by a catalytic triad of 3 aspartate residues (D268, D346, D447) surrounded by 15 arginine and lysine residues likely involved in transposase-target DNA interactions14. In addition to the hyperactive versions explored in the past9 we diversified additional residues that may influence PB excision and integration activities15 (SEQ 1-N). In order to isolate the best performing mutants, we developed a sensitive reporter system for targeted gene insertion, based on Emerald GFP (emGFP) reconstitution upon on-target integration. A promoterless C-terminal (C-t) half of emGFP preceded by a splicing acceptor was inserted in the genome of Hek293T cells to build a reporter cell line (dubbed as Hershey). A complementary ‘insertion-trap reporter’ was constructed encoding the N-terminal (N-t) first half of a emGFP with an upstream promoter followed by a splice donor and all flanked by the PB inverted repeats.

Specific gRNA-guided cas9 directed PB to insert the N-t half adjacent to the C-t half, which upon splicing of the resulting transcript leads to production of green fluorescence (FIG. 3). A library of cas9-PB chimeric proteins were assembled and transfected with the ‘insertion-trap reporter’ and guide-RNA (gRNA) to the cells containing the Hershey reporter. The variants presenting higher programmable insertion were tested separately using the same reporter cell line (FIG. 2a, 2b; 4a, 4b). This assay tested on-target activity (emGFP positive cells) and total transposition activity (RFP positive cells) using a dual transposon containing a N-t half emGFP and a full RFP sequence upstream. On-target to off-target ratio was calculated by dividing the percentage of emGFP positive cells (on-target activity) to the total percentage of RFP positive cells (total insertion activity).

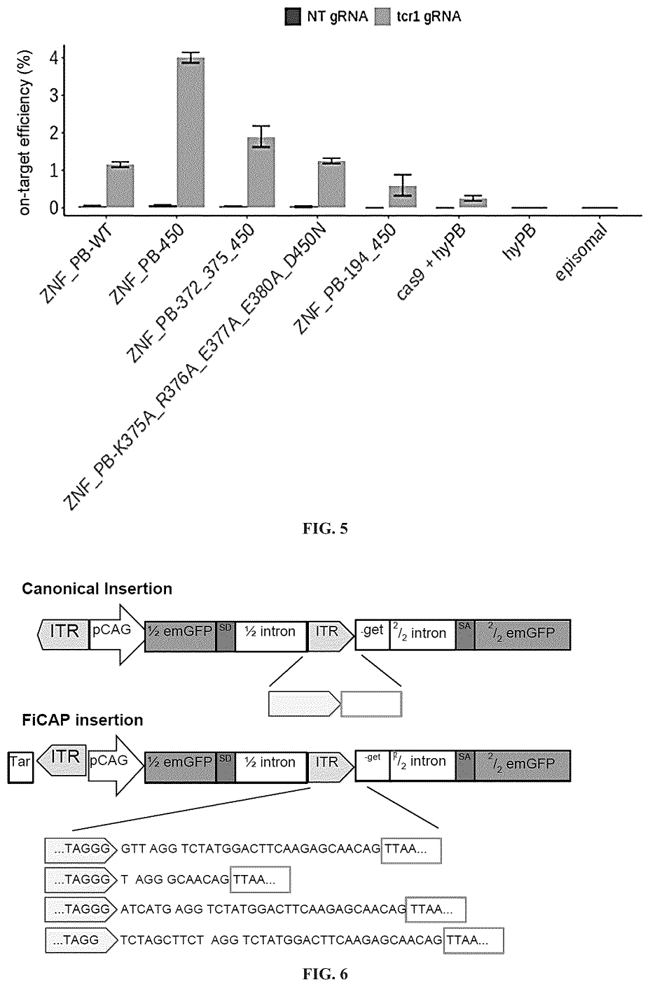

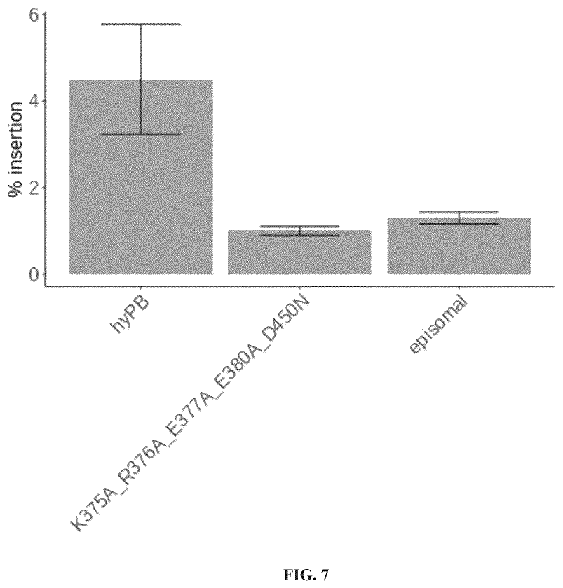

We tested various combinations of cas9 variants fused to PB variants for on-target and off-target transposition in the Hershey cell line. We observed the highest levels of programmable insertion in Nter-cas9-PB-Cter fusions containing i) cas9 with intact nuclease activity (FIG. 2A) and ii) PB variants with increased excision activity and iii) decreased t-DNA binding activity (FIG. 2B; 4A, 4B). We observed that all three parameters are important for achieving precise and efficient targeted insertion. First, the requirement for cas9 nuclease activity was demonstrated by the significantly lower efficiency of targeted insertion by ncas9 (D10A) and dcas9 (D10A and H840A) fused to PB (FIG. 2A). To further explore the role of the double-strand break (DSB) activity of cas9 in facilitating targeted integration, we uncoupled on-site targeting and DSB activity by using a Zinc finger-PB fusion for directed localization of the transposon and complemented it with on-site DSB by an independent Cas9 nuclease. Znf-PB fusion exhibited no or very low targeted insertion activity that was rescued when combined with introducing DSBs near the Znf binding site with gRNA guided-cas9 (FIG. 5). These results are consistent with a mechanism where DSB generation by cas9 in the vicinity of PB facilitates the insertional activity of PB and bypasses its requirement for the canonical TTAA at the insertion site. Indeed, our analysis of the cas9-PB insertion sites showed that Inverted terminal repeat (ITR) sequences get disrupted with the presence of small indels near the targeting site (FIG. 6). An important consequence of this disruption is the irreversibility of the PB-mediated integration mechanism. Mobilization of transposons with disrupted ITRs or TTAA is either eliminated or reduced16. This mechanism likely contributes to the efficiency of programmable insertion by cas9-PB, and the coupling of ‘find’ and ‘cut’ activity of cas9 with ‘pasting’ activity of modified PB contributes high levels of precision. Second, high excision PB mutants appear to contribute to higher programmable insertion (D450N, M194V; FIG. 2B, 4A, 4B). This enhanced excision may be a result of increased donor DNA substrate primed for integration. Furthermore, the destruction of the preferred substrate of excision may prevent enough excision even from PB mutants with higher excision activity (FIG. 6). Third, PB mutants with reduced t-DNA binding resulted in the lowest off-target levels (FIG. 2B, 4A, 4B). This result is consistent with a decrease in the intrinsic non-specific DNA binding by PB that is complemented by the sequence-specific DNA binding of cas9 in the cas9-PB fusion which inactivates PB solo insertion activity (FIG. 7).

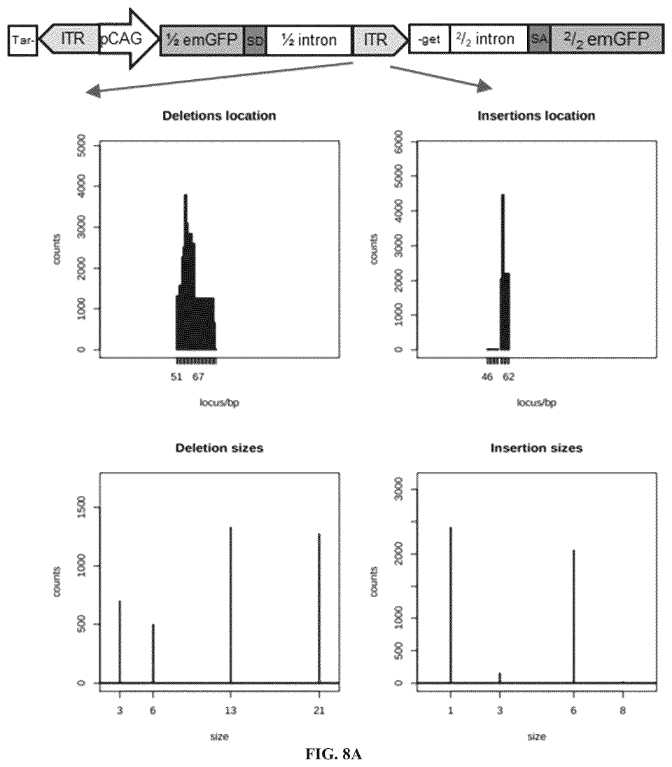

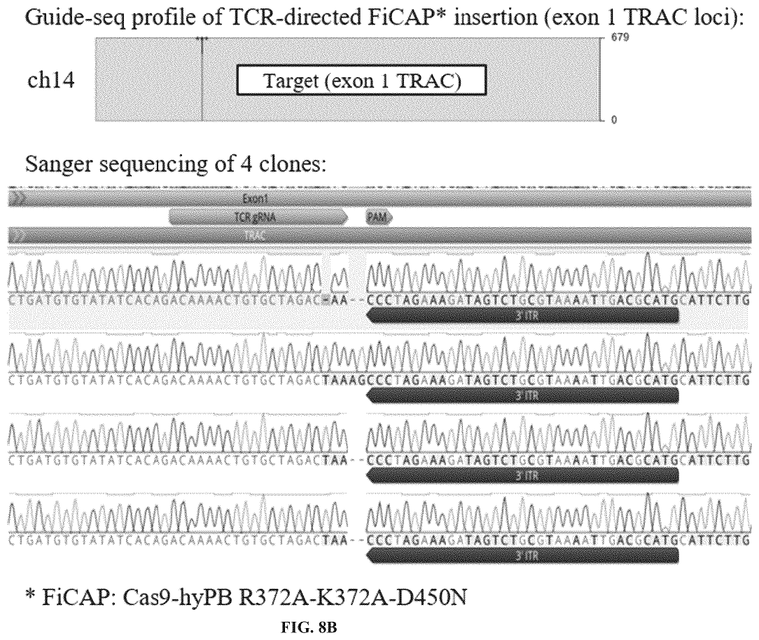

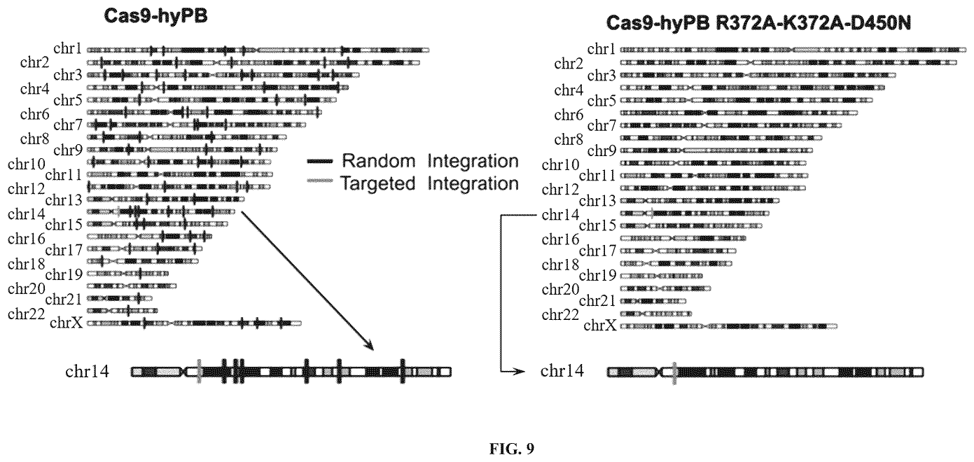

Using a genome guide approach, we were able to characterize programmable transposase insertion sites and off-target levels using a modified version of a Guide-seq17 based protocol (FIG. 8A). None of the on-target insertions detected happened at TTAA sites, further demonstrating integration on DSB sites generated by cas9 and resulting in the loss of preferred excision substrate (FIG. 8B). Additionally we run Guide-seq analysis on cells modified with programmable transposase technology targeting the TRAC loci. We detected all insertions on-target with sensitivity down to 1-10% (FIG. 9).

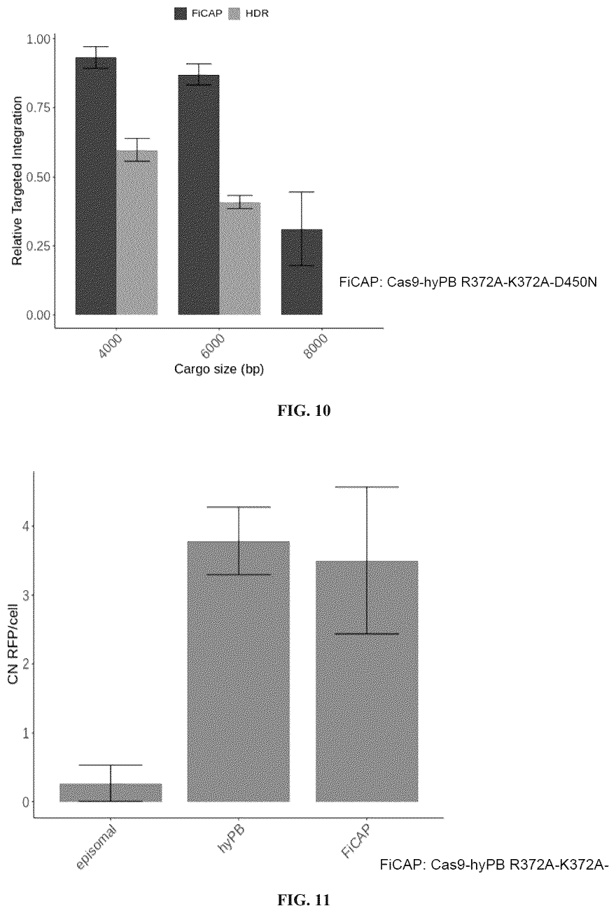

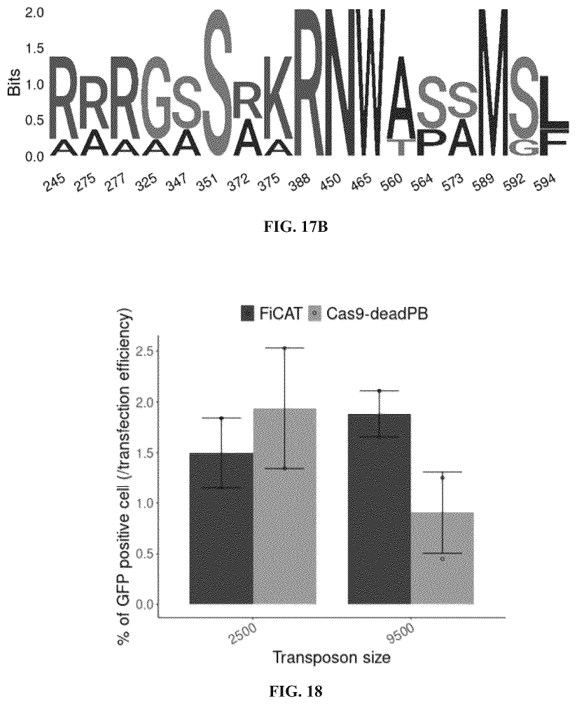

We have benchmarked programmable transposase technology with current methods for precise gene delivery such as cas9 based HDR (FIG. 10) programmable transposase shows higher efficiencies, a gap which widens in large payloads. The best mutants achieved insertions (up to 8 kb) with 2-fold more efficiency than HDR and high accuracy. We also compared programmable transposase with a HITI variant in which we fused Cas9 to a catalytic dead version of PB, which may help in recruiting DNA to the insertion site as it has been recently suggested by a similar approach using the DNA binding domain of the SB100 transposase. Programmable transposase presents twofold higher efficiency compared to alternative aided HITI methods (FIG. 18). With the aim to demonstrate programmable transposase activity in in vivo mouse models, we built mRNA versions of programmable transposase. We delivered programmable transposase to mice liver targeting Rosa26 genomic safe harbour using in vivo JetPEI reagent and observed high copy number of transgene compared to an endogenous gene (FIG. 11) and maintained transgene expression overtime (FIG. 20).

To sum up, we have coupled CRISPR molecular recognition and cleavage with DNA cut-and-paste activity of a modified PB to generate an efficient tool to perform precise and efficient gene delivery. This technology scales very well with payload size. We demonstrated its efficacy in Hek293T and mice liver. We envision programmable transposase technology as a generalized platform for therapeutic gene delivery for advanced therapies and other applications.

Results of Cas Variants Fused to hyPB

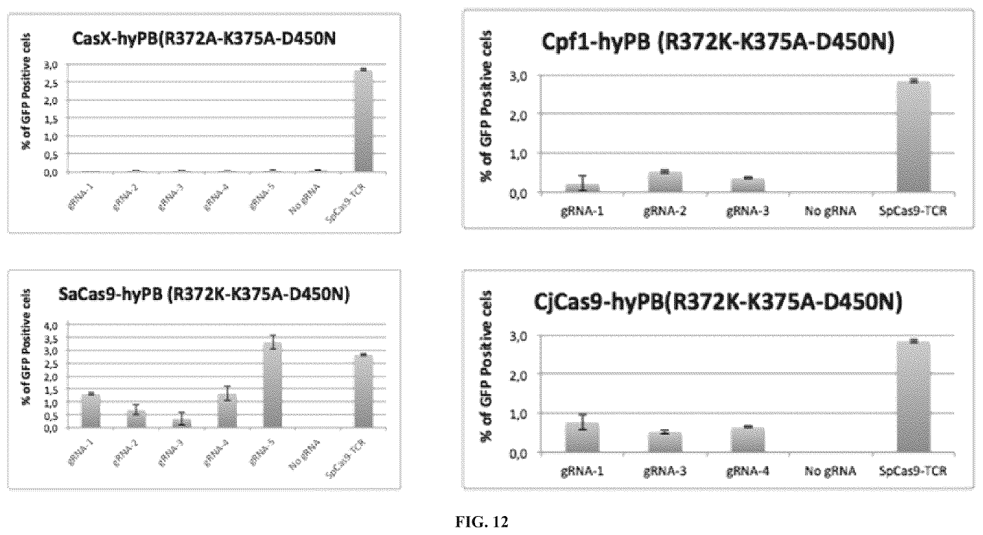

To further characterize the capacity of engineered hyPB to perform programmable transposition, we substituted the SpCas9 mudile of the programmable transposase tested, which other Cas proteins form different organisms with nuclease activity (namely SaCas9 of SEQ ID NO:72, cpf1 of SEQ ID NO:74, CasX of SEQ ID NO:75, and CjCas9 of SEQ ID NO:29). Specific gRNA targeting the region upstream of the split GFP reporter were designed and cloned for Hershey cell line transfection. (see Table 2 below) Targeted transposition was measured by means of GFP expression (FIG. 12).

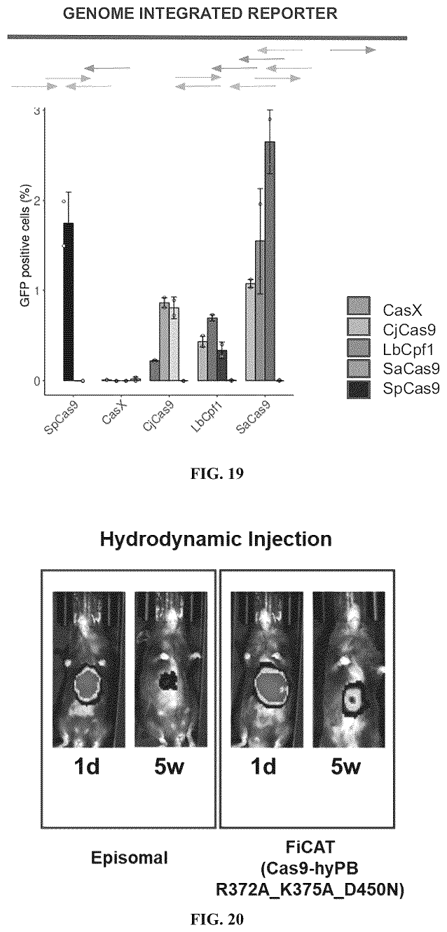

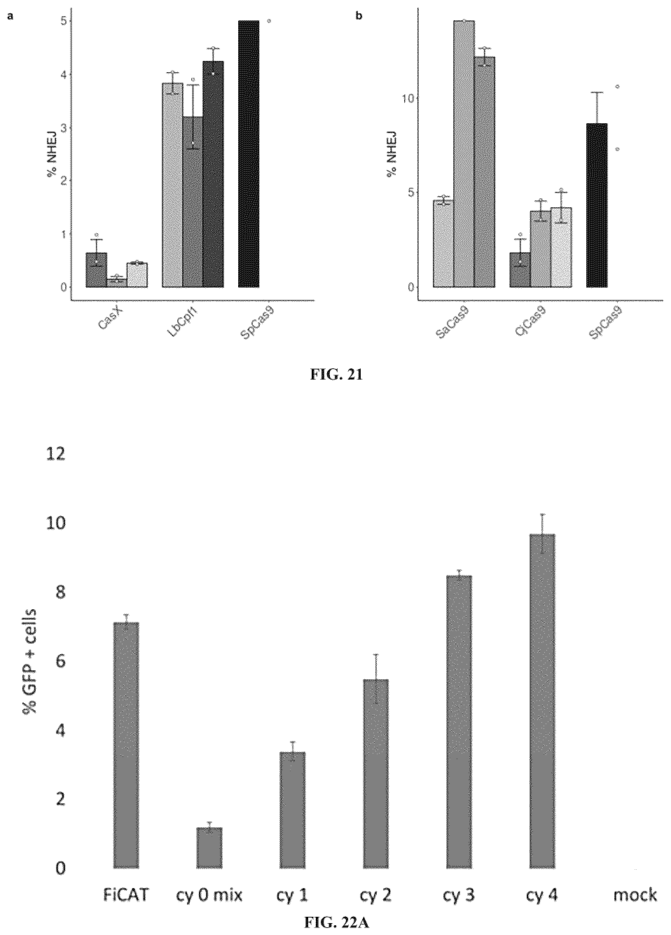

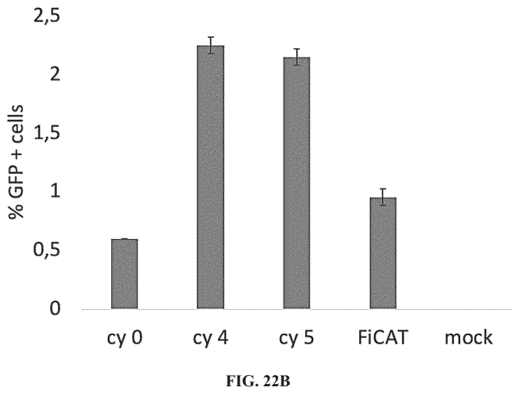

These results were confirmed in another line of experiments: we obtained good programmable insertion activity for CjCas9 and LbCpf1, while CasX did not achieve any programmable integration in our assay. Notably, SaCas9 had the highest levels of programmable insertion among the Cas proteins tested, with similar levels to SpCas9 fused to modified hyPB (FIG. 19). Indels were determined for the different Cas proteins used and the three different gRNA designed for each protein by Ilumina NGS (FIG. 21), shown for normalization purpose.

These positive results validate the engineered hyPB for programmable transposition to be useful for any sequence specific nuclease module.

Additional Results of Cas9 Fused to a Dimer of PB Mutants

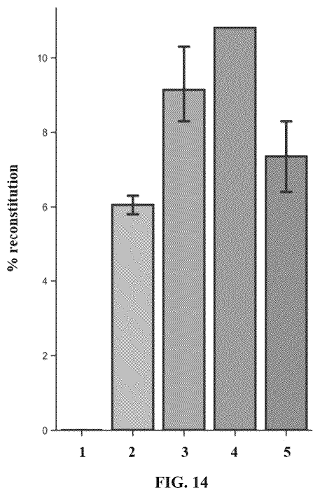

Given the nature of PB acting as dimers when performing transposition, we attempted to generate a fusion protein of Cas9 and hyPB R372A-K375A-D450N mutant. We compared the on target activity of these fusion to Cas9-PB mutants alone. We observed a better performance of the configuration Cas9-PB-PB; while the configuration PB-Cas9-PB did not outperform the Cas9-PB monomeric fusion (FIG. 14). For the dimeric fusion to Cas9 we used a recorded version of hyPB R372A-K375A-D450N mutant to facilitate cloning and expression.

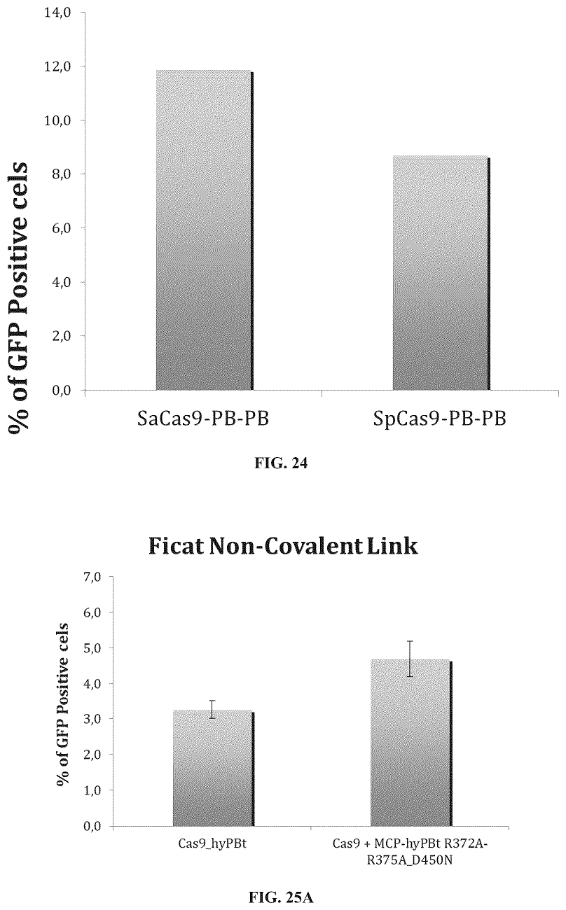

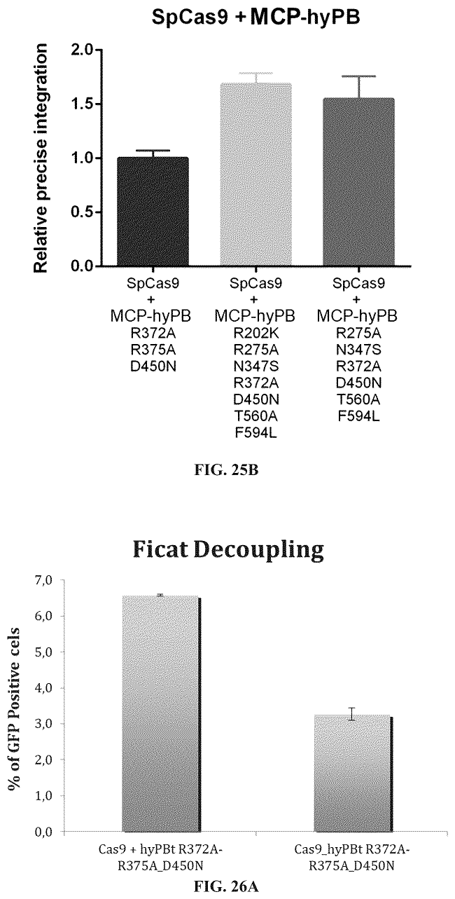

Interestingly, if the Cas9 fused to the dimeric hyPB R372A-K375A-D450N is the SaCas9 instead of SpCas9, the activity is further increased (FIG. 24). The increased performance of SaCas9 over SpCas9 with the dimeric hyPB is consistent with the results obtained with monomeric hyPB (FIGS. 12 and 19).

Results of ZNF-PB Mutants Rescue

We wanted to further explore the role of the double-strand break (DSB) activity induced by close by (4 nucleotides) target sites of two gRNAs promoting single stranded cuts by SpCas9 nickase variant (D10A) in facilitating targeted integration, while lowering off target activity by means of non-inducing DSB in off target sites. We used a Zinc finger-PB fusion for directed localization of the transposon, by fusion with D450N mutant and R372A-K375A-D450N mutant and complemented it with two on-site single stranded brakes by an independent nickase Cas9, or single DSB by Cas9 nuclease.

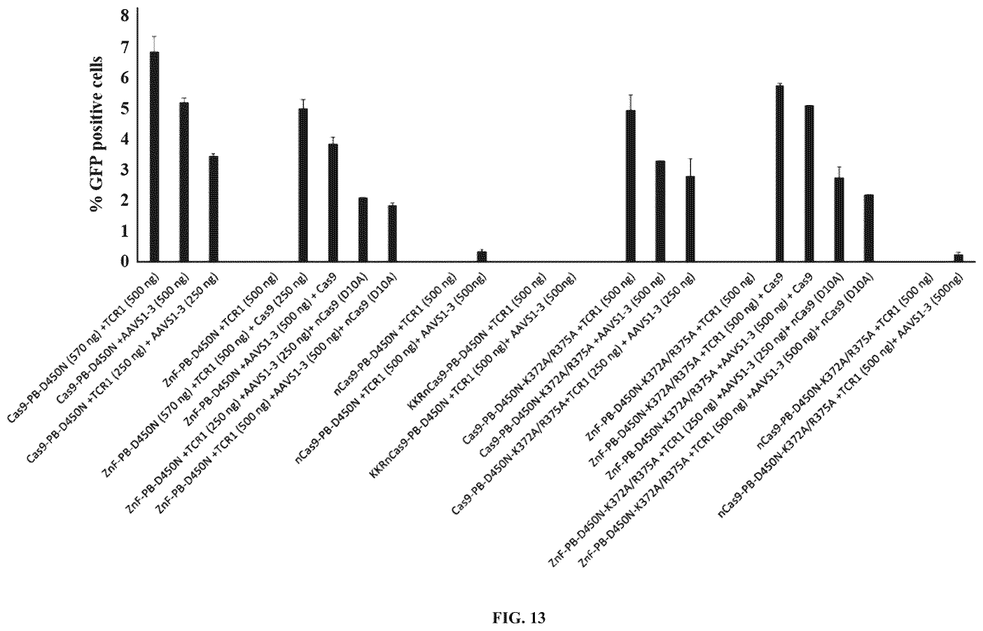

Znf-PB fusion exhibited no or very low targeted insertion activity that was rescued when combined with introducing DSBs near the Znf binding site with a single or dual gRNA guided-cas9 either nuclease or nickase (FIG. 13).

Results of Cas9-hyPB Mutant Variants

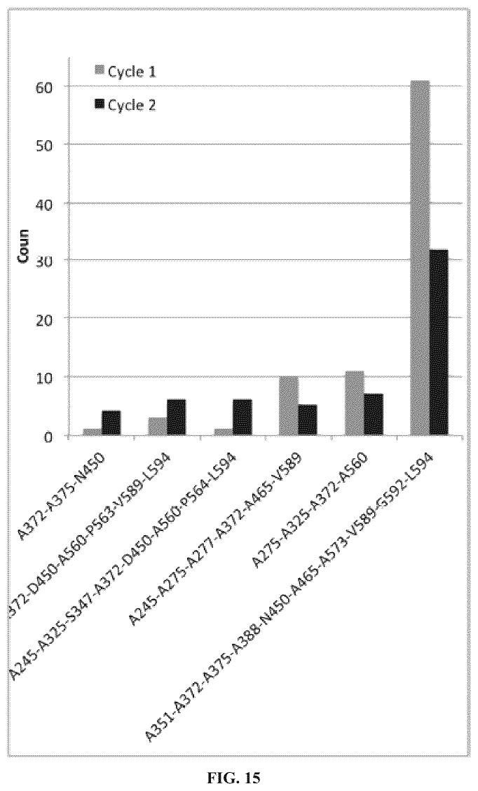

To further explore mutant combinations that could do programmable transposition with better efficiencies, several cycles of selection of cells were performed where GFP was reconstituted by programmable insertion of the split GFP reporter system. Interestingly we observed several combinations that out-performed the Cas9-hyPB R372A-K375A-D450N (FIG. 15). Especially worth of mention is variant of hyPB fused to Cas9 that are mutated on hyPB at AA: A351-A372-A375-A388-N450-A465-A573-V589-G592-L594 (also identified as SEQ ID NO:2), several fold enrich in the positive cells population compared to R372A-K375A-D450N (SEQ ID NO:1); and also A245-A275-A277-A372-A465-V589 (SEQ ID NO:3) and A275-A325-A372-A560 (SEQ ID NO:4) to a lesser extent.

In another line of experiments, PiggyBac DNA library was produced by Twist Bioscience, cloned in fusion with cas9 into a lentiviral vector and transformed into stb4 competent cells, ensuring ×100 variant complexity. Plasmids were purified by maxiprep and cotransfected with lentivirus packaging plasmids into Hek293T cells. Lentivirus was used to infect ½ GFP reporter cell line. Infected cells were transfected with the ½ GFP transposon and gRNA targeting AAVS1 sequence. GFP positive cells were selected by flow cytometry sorting and genomic DNA was extracted. PB was amplified from the extracted gDNA, recloned into lentiviral vector to restart a new cycle. Best performing programmable transposase variants were selected and transfected individually with AAVS1 gRNA and MC ½ GFP.

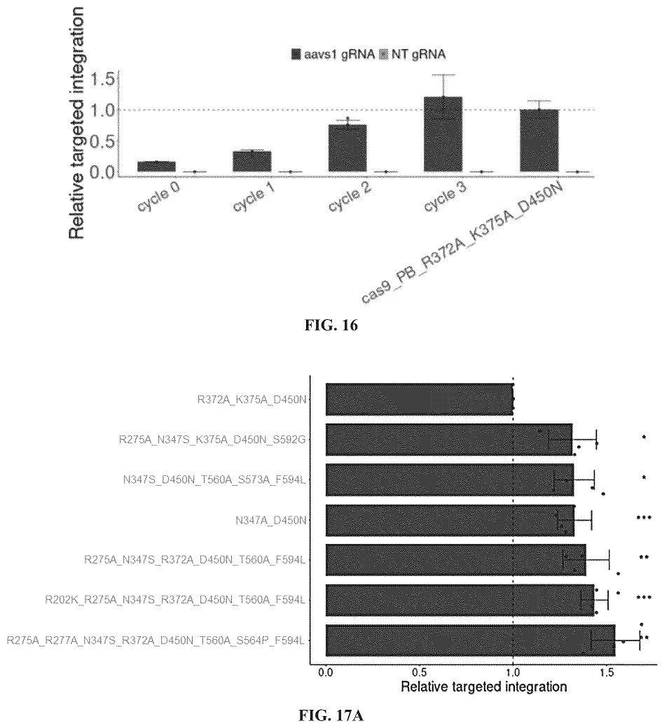

First, a random selection of 96 variants was performed and best performing variants were screened separately (FIG. 16). A summary of best PB amino acid variants for high on-target insertion confirms the importance of mutations D450N, R372A and K375A; but highlights other important residues which contribute to increased targeted efficiency (FIG. 17B). The six PB variants with best on-target efficiencies were selected (FIG. 17A). The individual on-target activities were significantly improved compared to FiCAT (Cas9-hyPB R372A-K375A-D450N) with the following variants: N347A-D450N; N347S-D450N-T560A-S573A-F594L; R202K-R275A-N347S-R372A-D450N-T560A-F594L; R275A-N347S-K375A-D450N-S592G; R275A-N347S-R372A-D450N-T560A-F594L; and R275A-R277A-N347S-R372A-D450N-T560A-S564P-F594L (two-sided t-test).

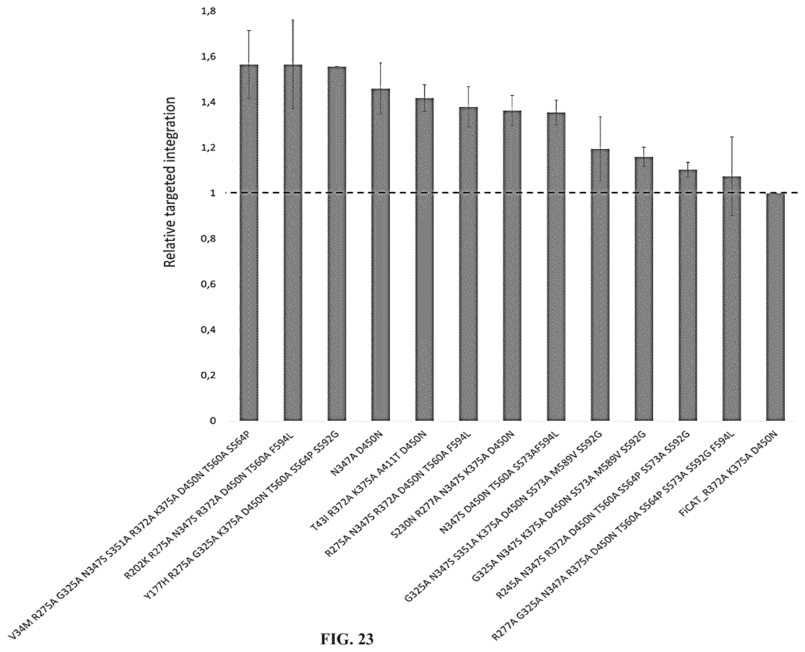

This experiment was repeated and confirmed (FIG. 22A). We also produced lentiviruses expressing bulk variants of each cycle and infected reporter cell line correcting its titer by the PB variants CN, demonstrating a similar increase of on-target efficiency over cycles (FIG. 22B). Single mutants were isolated from bulk variants after 4 and 5 cycles of cas9_PB library enrichment. Mutants were tested separately by transfecting on-target reporter cell line with FiCAT mutant, gRNA tcr1 and ½ GFP MC transposon. Best FiCAT mutants are shown in comparison with FiCAT R372A_K375A_D450N (FIG. 23). The individual on-target activities were significantly improved compared to FiCAT (Cas9-hyPB R372A-K375A-D450N) with the following variants: R202K-R275A-N347S-R372A-D450N-T560A-F594L; R245A-N347S-R372A-D450N-T560A-S564P-S573A-S592G; R275A-N347S-R372A-D450N-T560A-F594L; N347A-D450N; R277A-G325A-N347A-K375A-D450N-T560A-S564P-S573A-S592G-F594L; N347S-D450N-T560A-S573A-F594L; V34M-R275A-G325A-N347S-S351A-R372A-K375A-D450N-T560A-S564P; G325A-N347S-K375A-D450N-S573A-M589V-S592G; S230N-R277A-N347S-K375A-D450N; T43I-R372A-K375A-A411T-D450N; G325A-N347S-S351A-K375A-D450N-S573A-M589V-S592G; Y177H-R275A-G325A-K375A-D450N-T560A-S564P-S592G.

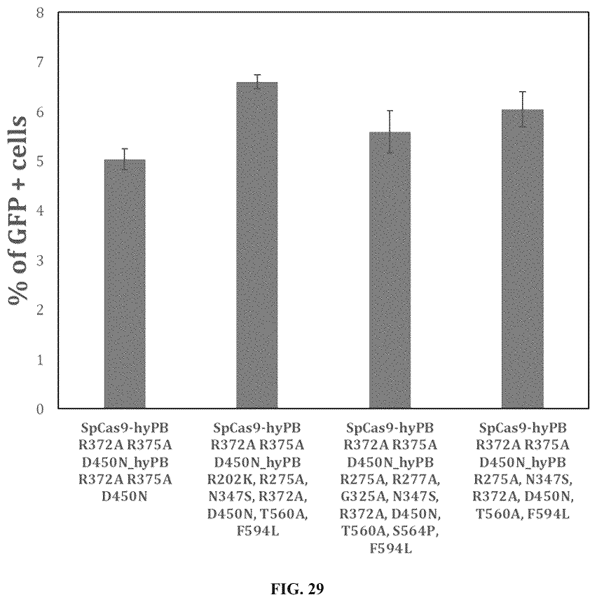

The superiority of mutants R202K-R275A-N347S-R372A-D450N-T560A-F594L, R275A-R277A-N347S-R372A-D450N-T560A-S564P-F594L and R275A-N347S-R372A-D450N-T560A-F594L compared to the mutant R372A-K375A-D450N was further demonstrated in triple fusion proteins comprising a SpCas9 and two hyPB (FIG. 29).

Results of Cas9 and hyPB Non-Covalent Linking

In addition to the covalent binding of Cas9 with hyPB R372A-K375A-D450N through a linker, we user the MS2-MCP system to link Cas9 and the fusion protein consisting of MCP protein and hyPB R372A-K375A-D450N through a modified gRNA containing a tetraloop of MS2 sequence binding the MCP protein.

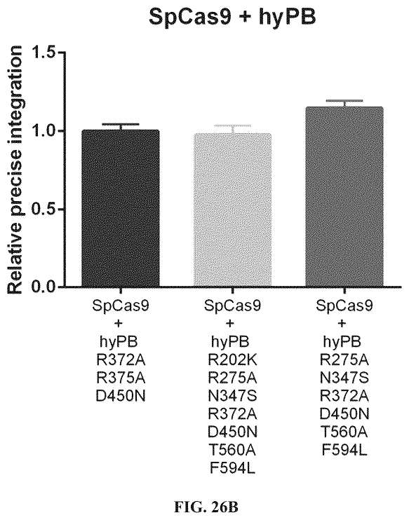

The combination of MCP-hyPB R372A-K375A-D450N fusion protein with Cas9 had an increased programmable insertion activity compared to Cas9-hyPB R372A-K375A-D450N fusion protein (FIG. 25A). In addition, we fused the MCP protein to other mutants of hyPB to perform programmable transposition in combination with SpCas9. Both variants used (R202K-R275A-N347S-R372A-D450N-T560A-F594L, and R275A-N347S-R372A-D450N-T560A-F594L) outperformed R372A-K375A-D450N (FIG. 25B).

Results of Cas9 and hyPB Decoupling for Programmable Transposition

We also tried the performance of SpCas9 with hyPB R372A-K375A-D450N without a linker, nor the MS2-MCP system. We co-expressed in the same cells SpCas9 and hyPB R372A-K375A-D450N, and an increased programmable insertion activity was registered compared to Cas9-hyPB R372A-K375A-D450N fusion protein (FIG. 26A). We extended the number of hyPB mutant variant tested not fused to Cas9, but being expressed at the same time, and acting together to achieve the activity of programmable transposition (FIG. 26B).

Results of Co-Expression of Cas9-hyPB and MCP-hyPB Fusion Proteins

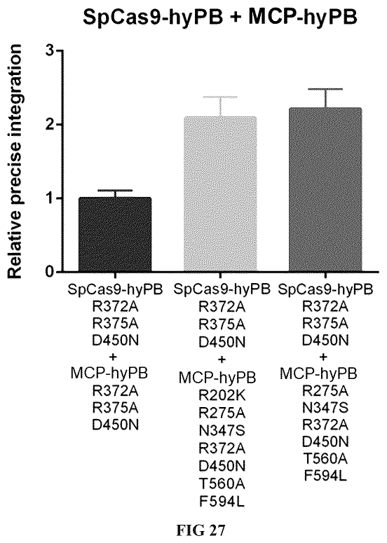

We co-transfected hyPB R372A-K375A-D450N mutant fused to MCP protein, and hyPB mutants fused to SpCas9, in order to obtain a dimeric version of the fusion with one of the monomers being non-covalently linked. Several hyPB mutants fused to SpCas9 were compared for specific target integration (FIG. 27).

Results of Co-Expression of Cas9-hyPB and hyPB Variants

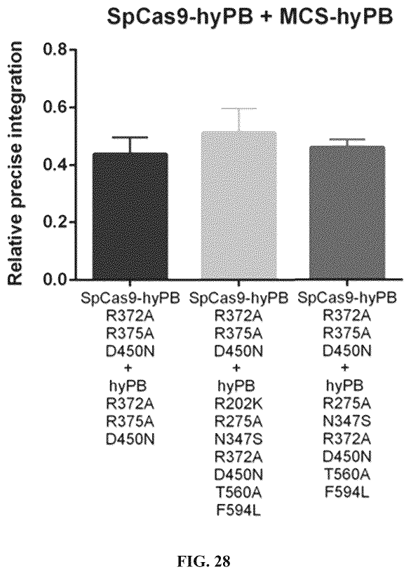

In a similar manner, we co-transfected the SpCas9 hyPB R372A-K375A-D450N fusion protein and hyPB mutants independently, in order to obtain a dimeric version of the fusion with one of the monomers not linked (FIG. 28).

Methods

Cloning and plasmids. RFP transposon PB512-B for random insertion monitoring was purchased from System Biosciences Inc. hyPB vector was obtained from Wellcome Trust Sanger Institute (pCMV_hyPBase)9. Plasmid vector pCRTM-Blunt TI-TOPO® was from Invitrogen and cas9, ncas9 and SP-dcas9-VPR were obtained from Addgene (Addgene plasmid #41815, #41816, #63798). Finally, SB100X and pT4-HB were a kind gift from Dr. Zsuzsana Zizsvak. gRNAs were produced using The Zero Blunt TOPO PCR cloning kit (Invitrogen). with a gblock gene fragment (Integrated DNA Technologies) containing U6 promoter, 20 nt target site, gRNA scaffold and terminator. gRNA TRAC was designed and validated in the lab and gRNA aavs1 3 sequence was previously described18.

Nuclease, nickase and dead cas9 fusions to hyPB and PB RFP ½ emGFP SMN1 transposon were performed by Golden Gate assembly using BspQI enzyme and standard methods.

Different mutations were introduced into hyPB sequence fused to cas9 (cas9_PB plasmid) by site directed mutagenesis following Quickchange Lightning mutagenesis kit's instructions (Agilent). Primers were designed with QuickChange Primer Design to achieve following mutations to the hyPB sequence: M194V, R245A, G325A, R372A, K375A, R376A, E377A, E380A, D450N, S564P (referring to SEQ ID NO:90-99). All plasmids are available upon request. PB ½ emGFP SMN1 was obtained by introducing the first half of emGFP sequence and SMN1 intron 6 sequence into piggyBac acceptor vector. pT4 SMN1 2/2 emGFP was obtained by adding a second half SMN1 intron 6 and partial emGFP in SB100X transposon vector. emGFP sequences containing SMN1 were obtained from DYP004reporter19, a kind gift from Sri Kosuri.

Transposon and HDR templates of different sizes were generated by cloning a partial cDNA (NC_000006.12) fragment upstream of the split emGFP reporter system

Cell culture, transfection and electroporation. Hek293T cell line (Thermo Fisher Scientific) and C2C12 cell line (ATCC) were cultured at 37° C. in a 5% CO2 incubator with Dulbecco's modified eagle medium (DMEM), supplemented with high glucose (Gibco, Therm Fisher), 10% Fetal Bovine Serum (FBS), 2 mM glutamine and 100 U penicillin/0.1 mg/mL streptomycin. Jurkat cell line was cultured at 37° C. in a 5% CO2 incubator with Roswell Park Memorial Institute 1640 medium (RPMI) supplemented with Glutamax and HEPES (Gibco, Thermo Fisher) and 10% FBS. This cell line was a gift from Manel Juan (Hospital Clinic, Barcelona). Hek293T cell's transfection experiments were performed using lipofectamine 3000 reagent following manufacturer's instructions or Polyethyleneimine (PEI, Thermo Fisher Scientific) at 1:3 DNA-PEI ratio in OptiMem. Cells were seeded the day before to achieve 70% confluency on transfection day (usually 290.000 cells in adherent p12 well plate). Plasmid DNA ratio was 1 transposase: 2.5 gRNA: 2.5 transposon or 1 Cas9: 2.5 gRNA: 2.5 HDR template using either 0.076 pmols programmable transposase or Cas9 for p12 well plate.

emGFP splicing based reconstitution Assay. Hek293T cell line containing pT4 SMN1 2/2 emGFP was generated by PEI mediated transfection of SB100X and pT4 SMN1 2/2 emGFP DNA constructs, followed by single clone expansion and PCR genotyping (Supplementary Table 3). A positive clone was selected and expanded and used for subsequent assays. For emGFP reconstitution assay, programmable transposase, gRNA and transposon plasmids were transfected in a 1 programmable transposase: 2.5 gRNA: 2.5 transposon ratio using 0,076 pmol programmable transposase or hyPB and 0.19 pmols transposon and gRNA for a 12 wells plate. On-target insertion was measured 5 days post-transfection by emGFP fluorescence. Off-target transposition was measured 15 days post-transfection by RFP fluorescence. On target insertion was measured by cotransfecting PB variant, gRNA and PB % SMN1 RFP emGFP (insert definitive construct name) expression plasmids and determining emGFP and RFP fluorescence after 14 days (set average n of days for episomal decay) by emGFP expression measured at (BD LSR Fortessa; BD Biosciences. Blue 488 nm laser with 530/30 filter and Yellow Green 561 nm laser with 610/20 filter)

Junction PCRs for insertion site sequencing Junction PCR was performed on emGFP sorted cells with BD FACSAria (Biosciences). Selected cells had on-target insertion of PB ½ emGFP SMN1 transposon targeting TRAC target site on reporter cell line. Genomic DNA was extracted using DNeasy Blood and tissue kit (Qiagen). Primers were designed by the 3′ ITR of the transposon (forward) and targeting the intron of the 2/2 emGFP of the reporter cell line or the endogenous T cell receptor (TRAC) (reverse) (Supplementary Table 4).

Bioinformatics analysis of Guide-SEQ experiment. Illumina reads were clustered with usearch20 and mapped to the reference using bwa-mem21. For on-target insertion characterization, reads covering 5′ and 3′ junctions from the target insertion site were selected with Python scripting and Samtools22. Number of indels was obtained with CRISPR-GA23. For on-target and off-target experiments, clustered reads that mapped against the vector were selected and mapped against the reference genome using bwa-mem. Significance of the insertion peaks was assessed with macs224 algorithm.

Guide-seq library prep adapted to targeted insertion. An adapted Guide-seq¹⁵ protocol implementation was performed by extracting genomic DNA using DNeasy Blood and tissue kit (Qiagen) and fragmented to 500 bp fragments using Q800R3 Sonicator. End repair, A-tailing, and ligation of Y-adapter were performed using KAPA Hyper Prep Kit (KR0961-v5.16) and 3 ug of fragmented genomic DNA, followed by AMPure XP SPRI bead purification at 1× ratio. After adapter ligation, each sample was split in two and amplified with GSP5′ or GSP3′ to capture 5′ and 3′ junctions, respectively. To capture 5′ and 3′ transposon-genome junctions, two nested PCRs were performed using KAPA HiFi DNA Polymerase following manufacturer protocol: PCR1 with P5_1 and PB_5_GSP1 or PB_3_GSP1 in a 25 ul final volume; and PCR2 with P5_2 PB_5_GSP2 or PB_3_GSP2 in a 25 ul final volume. 5′ and 3′ PCR products were purified with AMPure XP SPRI bead purification at 1× ratio, mixed in equimolar ratio and sequenced with Illumina Miseq Reagent Kit V2-500 cycle (2×250 bp paired end). 3 ul of 100 μM custom primers index 1 and Read 2 were added to the sequencing reaction.

in vivo targeted insertion to mice liver. Animal experimentation procedures were approved by the Animal Experimentation Ethic Committee of Barcelona Biomedical Research Park. C57BL/6J, 8-10 weeks old were used for this study. The animals were purchased from Jackson Laboratories, male and female were used without distinction. programmable transposase mRNA was produced with RiboMAX Large Scale RNA Production Systems-T7 (Promega) following manufacturer's instructions. Rosa26 gRNA25 was purchased from IDT. programmable transposase mRNA, gRNA targeting Rosa26 and PB512-B transposon were injected via retro-orbital in a 1:1:2.5 ratio. A total of 60 ug of nucleic acids were complexed to; In vivo-JetPEI (Polyplus transfection) at NP ratio 7. Animals were euthanized 10 days after-injection and liver was isolated and homogenized. Genomic DNA was extracted from liver samples with DNeasy Blood and tissue kit (Qiagen) Transposon relative Copy number to Tfrc endogenous gene was obtained by qPCR (primers listed in Supplementary Table 1). Imaging of luciferase expression was performed at different timepoints after FiCAT-gRNA-transposon or transposon control administration with IVIS spectrum imaging system (Caliper Life Sciences). Images were taken 5 min after intraperitoneal injection of D-Luciferin potassium salt (Gold Biotechnology) according to the manufacturer's instructions.

PB structural modelling. A 3D structure of the Trichoplusia ni piggyBac transposase protein was obtained by Robetta Web protein structure prediction server (http://robetta.bakerlab.org). The core domain (131-550aa) was predicted by Rosetta Comparative Modelling method that is based on Monte Carlo algorithm with embedded Cartesian-space minimization and all-atom optimization26. The tertiary structure fold was analysed and validated with SPServer and ProSa-Web knowledge-based methods (Supplementary FIG. 2). Secondary structure was analysed with PSIPRED and HHPred machine-learning based methods. PB's core was then modelled for refinements with PyMOL by comparative protein modelling methods. The refinement process was guided by the superimposition of the piggyBac model with Cryo-EM HIV-1 Strand Transfer Complex Intasome (PDB ID: 5U1C) consisting of the HIV integrase tetramer bound to viral DNA and target host DNA and X-ray diffraction Tn5 transposase complex structure (PDB ID: 1MUS27). Strand-transferring DNA and donor DNA were extrapolated from the superimpositions of HIV-1 Intasome and Tn5 respectively. The nucleotides in the interface in contact with the protein were analyzed with X3DNA as double-strand DNA. We used statistical potentials to score the interaction between protein and DNA and generate a theoretical PWM28. The theoretic PWM is obtained by testing all potential double-strand DNA sequences in the interface, ranking them with the statistical potentials and selecting the top to make a multiple sequence alignment. During the submission of this manuscript a cryo-EM structure became available, which shows important agreement with modelling performed29. Cryo-EM structure of piggyBac transposase strand transfer complex (PDB ID: 6X67) confirmed the general fold of the model and the domains we hypothesized were responsible for the contact with donor and target DNA.

TABLE 2
Cas variants gRNA

| Cas | | |
|---|---|---|
| Variant | Sequence | PAM |
| SaCas9 | TATGTACACTTCTGACCCAC (SEQ ID NO: 113) | TGGAAT |
| | GCCTTTAAGCTTGATATCCA (SEQ ID NO: 114) | TGGAAT |
| | GTATCACAATTCCAGTGGGT (SEQ ID NO: 115) | CAGAA |
| | GGACAGGATCGGCATAACCG (SEQ ID NO: 116) | GTGAAT |
| | GTGCTCGGGGCCACTAGGGA (SEQ ID NO: 117) | CAGGAT |
| Cpf1 | ACTTATAATTCACTGTATCA (SEQ ID NO: 118) | TTTC |
| | AGCTTGATATCCATGGAATT (SEQ ID NO: 119) | TTTA |
| | TGCTCGGGGCCACTAGGGAC (SEQ ID NO: 120) | TTTG |
| | CTTTTGTAAAACTTTATGGT (SEQ ID NO: 121) | TTTA |
| | CAAAAGTAAATAGCCCGGCT (SEQ ID NO: 122) | TTTA |
| CjCas9 | GCCGATCCTGTCCCTAGTGGCC (SEQ ID NO: 123) | CCGAGCAC |
| | ACAATTCCAGTGGGTCAGAAGT | GTACATAC |
| | (SEQ ID NO: 124) | |
| | ACACTTCTGACCCACTGGAAT (SEQ ID NO: 125) | TGTGATAC |
| | GAATTCCATGGATATCAAGCTT (SEQ ID NO: 126) | AAAGGCA |
| | | C |
| | AATTCCAGTGGGTCAGAAGTGT | ACATACAC |
| | (SEQ ID NO: 127) | |
| CasX | TCAAGCGCGTGTATGTACAC (SEQ ID NO: 128) | TTCT |
| | GGATCGGCATAACCGGTGAA (SEQ ID NO: 129) | TTCC |
| | TAGACATGAGGTCTATGGAC (SEQ ID NO: 130) | TTCA |
| | TAAGCTTGATATCCATGGAA (SEQ ID NO: 131) | TTCA |
| | TATAATTCACTGTATCACAA (SEQ ID NO: 132) | TTCC |

### REFERENCES

1. Porteus, M. H. & Carroll, D. Nat. Biotechnol. 23, 967-973 (2005).
2. Sander, J. D. & Joung, J. K. Nat. Biotechnol. 32, 347-355 (2014).
3. Rees, H. A. & Liu, D. R. Nat. Rev. Genet. doi:10.1038/s41576-018-0059-1
4. Anzalone, A. V. et al. Nature 576, 149-157 (2019).
5. He, X. et al. Nucleic Acids Res. 44, e85 (2016).
6. Suzuki, K. et al. Nature 540, 144-149 (2016).
7. Klompe, S. E., Vo, P. L. H., Halpin-Healy, T. S. & Sternberg, S. H. Nature 1 (2019).
8. Strecker, J. et al. Science (2019).doi:10.1126/science.aax9181
9. Yusa, K., Zhou, L., Li, M. A., Bradley, A. & Craig, N. L. Proc. Natl. Acad. Sci. U.S.A 108, 1531-1536 (2011).
10. Hew, B. E., Sato, R., Mauro, D., Stoytchev, I. & Owens, J. B. Synth. Biol. 4, ysz018 (2019).
11. Kovač, A. et al. Elife 9, (2020).
12. Loperfido, M. et al. Nucleic Acids Research 44, 744-760 (2016).
13. Passos, D. O. et al. Science 355, 89-92 (2017).
14. Li, X. et al. doi:10.1073/pnas.1305987110
15. Morellet, N. et al. Nucleic Acids Res. 46, 2660-2677 (2018).
16. Li, M. A. et al. Mol. Cell. Biol. 33, 1317-1330 (2013).
17. Tsai, S. Q. et al. Nat. Biotechnol. 33, 187-197 (2015).
18. Mali, P. et al. Science 339, 823-826 (2013).
19. Cheung, R. et al. Molecular Cell 73, 183-194.e8 (2019).
20. Edgar, R. C. Bioinformatics 26, 2460-2461 (2010).
21. Li, H. arXiv [q-bio.GN](2013).at <http://arxiv.org/abs/1303.3997>
22. Li, H. et al. Bioinformatics 25, 2078-2079 (2009).
23. Guell, M., Yang, L. & Church, G. M. Bioinformatics 30, 2968-2970 (2014).
24. Gaspar, J. M. 496521 (2018).doi:10.1101/496521
25. Chu, V. T. et al. BMC Biotechnol. 16, 4 (2016).
26. Fu, D. Y. (2018) at <https://etd.library.vanderbilt.edu/available/etd-08012018-164524/unrestricted/DarwinYFu_Thesis_Submit.pdf>
27. Steiniger-White, M., Rayment, I. & Reznikoff, W. S. Curr. Opin. Struct. Biol. 14, 50-57 (2004).
28. Meseguer, A. et al. NAR Genom Bioinform 2, (2020).
29. Chen, Q. et al. Nat. Commun. 11, 3446 (2020).

1. A composition comprising
(i) a first protein comprising or consisting of a site-specific DNA binding protein capable of binding and cleaving a target nucleic acid sequence; or a nucleic acid construct encoding said first protein; and
(ii) a second protein comprising or consisting of a transposase; or a nucleic acid construct encoding said second protein;
wherein said transposase is a modified hyperactive PiggyBac, comprising one or more amino acid mutations as compared to hyperactive PiggyBac of SEQ ID NO: 9.

2. The composition according to claim 1, wherein the first protein and the second protein are fused together to form a fusion protein.

3. The composition according to claim 1, wherein the first protein is fused to the C-terminal end of the second protein.

4. The composition according to claim 1, wherein said transposase is a modified hyperactive PiggyBac, comprising one or more amino acid mutations to increase excision activity as compared to unmodified hyperactive PiggyBac, and/or one or more amino acid mutations to decrease DNA binding activity as compared to unmodified hyperactive PiggyBac.

5. The composition according to claim 1, wherein said one or more amino acid mutations do not consist of R372A, K375A, and D450N.

6. The composition according to claim 1, wherein said one or more amino acid mutations are selected among the amino acid substitutions which increase excision activity at position of M194, D450, T560, 5564 S573, S592 or F594, said position number corresponding to the amino acid number of unmodified hyperactive PiggyBac of SEQ ID NO: 9.

7. The composition according to claim 1, wherein said one or more amino acid mutations are selected among the amino acid substitutions which increase excision activity at position of M194 or D450, said position number corresponding to the amino acid number of unmodified hyperactive PiggyBac of SEQ ID NO: 9.

8. The composition according to claim 1, wherein said one or more amino acid mutations are selected among the amino acid substitutions which decrease DNA binding activity at position R275, R277, R347, R372, K375, R376, E377, and/or E380, said position number corresponding to the amino acid number of unmodified hyperactive PiggyBac of SEQ ID NO: 9.

9. The composition according to claim 1, wherein said one or more amino acid mutations are selected among the amino acid substitutions which decrease DNA binding activity at position R372, K375, R376, E377, and/or E380, said position number corresponding to the amino acid number of unmodified hyperactive PiggyBac of SEQ ID NO: 9.

10. The composition according to claim 1, wherein the modified hyperactive PiggyBac includes the double mutations N347S and D450N, said position number corresponding to the amino acid number of unmodified hyperactive PiggyBac of SEQ ID NO: 9.

11. The composition according to claim 1, wherein the modified hyperactive PiggyBac mutation comprises one of the following amino acid substitution or combination of amino acid substitutions: R372A/K375A/R376A/D450N, K375A/R376A/E377A/E380A/D450N, R372A/K375A/R376A/E377A/E380A/D450N, M194V, R376A, E377A, E380A, M194V/R372A/K375A, S351A/R372A/K375A/R388A/D450N/W465A/S573A/M589V/S592G/F594L, R245A/R275A/R277A/R372A/W465A/M589V, R275A/325A/R372A/T560A, N347A/D450N, N347S/D450N/T560A/S573A/F594L, R202K/R275A/N347S/R372A/D450N/T560A/F594L, R275A/N347S/K375A/D450N/S592G, R275A/N347S/R372A/D450N/T560A/F594L, R275A/R277A/N347S/R372A/D450N/T560A/S564P/F594L, R245A/N347S/R372A/D450N/T560A/S564P/S573A/S592G, R277A/G325A/N347A/K375A/D450N/T560A/S564P/S573A/S592G/F594L, V34M/R275A/G325A/N347S/S351A/R372A/K375A/D450N/T560A/S564P, G325A/N347S/K375A/D450N/S573A/M589V/S592G, S230N/R277A/N347S/K375A/D450N, T43I/R372A/K375A/A411T/D450N, G325A/N347S/S351A/K375A/D450N/S573A/M589V/S592G, Y177H/R275A/G325A/K375A/D450N/T560A/S564P/S592G; the position number corresponding to the amino acid number of the hyperactive PiggyBac of SEQ ID NO: 9, typically said modified transposase has an amino acid sequence selected among any of SEQ ID NO: 2-8, 10-18 and 135-149.

12. The composition according to claim 1, further comprising a third protein comprising or consisting of a second transposase; or a nucleic acid construct encoding said third protein; wherein said second transposase is either an hyperactive PiggyBac with SEQ ID NO: 9, or a modified hyperactive PiggyBac with comprising one or more amino acid mutations as compared to the hyperactive PiggyBac with SEQ ID NO: 9.

13. The composition according to claim 12, wherein the first, second and third proteins are fused together to form a triple fusion protein.

14. The composition according to claim 1, wherein the first protein comprises or consists of an RNA-guided nuclease or nickase, or a zinc finger nuclease.

15. The composition according to claim 1, wherein said first protein is a nuclease protein comprising an active DNA cleavage domain and a guide RNA binding domain and having at least 80%, 90%, 95%, 99% or at least 100% identity to a Streptococcus pyogenes Cas9 (SpCas9) of SEQ ID NO: 31, Staphylococcus aureus Cas9 (SaCas9) of SEQ ID NO: 72, Cpf1 of SEQ ID NO: 74, Campylobacter jejuni Cas9 (CjCas9) of SEQ ID NO: 29, Streptococcus pyogenes Cas9 nickase (nCas9) of SEQ ID NO: 70, CasX of SEQ ID NO: 75, or Staphylococcus aureus Cas9 nickase of SEQ ID NO: 76.

16. The composition according to claim 1, further comprising a guide RNA, and an exogenous nucleic acid for insertion in a genome.

17. The composition according to of claim 16, wherein the transposase is fused to an RNA-binding protein capable of binding to at least one specific RNA sequence comprised in the guide RNA.

18. The composition according to claim 16, wherein said exogenous nucleic acid is a large DNA fragment, typically having a size between 5 kb and 25 kb.

19. (canceled)

20. A nucleic acid encoding a fusion protein as defined in claim 2, typically a messenger RNA (mRNA).

21. An in vitro method for site specific integration of an exogenous nucleic acid sequence into the genome of a cell, the method comprising delivering to the cell the composition according to claim 1, a guide RNA, and the exogenous nucleic acid.

22. (canceled)


SEQUENCE LISTING (153 sequences)

SEQ ID NO: 1 - protein, 594 aa, Artificial Sequence
  Modified Hyperactive Piggybac
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 2 - protein, 594 aa, Artificial Sequence
  Modified hyperactive PiggyBac
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTAIPLAKNLLQEPYKLTIVGTVASNAREIPEVLKNSRSAPVGTSMFCFDGPLTLVSYKPKPAKMVYLLSSCDEDASINESTGKPQMVMYYNQTKGGVDTLNQMCSVMTCSRKTNRAPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASAACKKCKKVICREHNIDVCQGCL

SEQ ID NO: 3 - protein, 594 aa, Artificial Sequence
  Modified Hyperactive Piggy
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVAKIWDLFIHQCIQNYTPGAHLTIDEQLLGFAGACPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLDQMCSVMTCSRKTNRAPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDVCQSCF

SEQ ID NO: 4 - protein, 594 aa, Artificial Sequence
  Modified hyperactive PiggyBac
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFAGRCPFRVYIPNKPSKYGIKILMMCDSGTKYMINGMPYLGRGTQTNGVPLAEYYVKELSKPVHGSCRNITCDNWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPSKIRRKASASCKKCKKVICREHNIDMCQSCF

SEQ ID NO: 5 - protein, 594 aa, Artificial Sequence
  Modified hyperactive PiggyBac
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTAIPLAKNLLQEPYKLTIVGTVASNAREIPEVLKNSRSAPVGTSMFCFDGPLTLVSYKPKPAKMVYLLSSCDEDASINESTGKPQMVMYYNQTKGGVDTLNQMCSVMTCSRKTNRAPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASAACKKCKKVICREHNIDVCQRCL

SEQ ID NO: 6 - protein, 594 aa, Artificial Sequence
  Modified hyperactive PiggyBac
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFAGACPFRVYIPNKPSKYGIKILMMCDSGTKYMINGMPYLGRGTQTNGVPLGEYYVKELSKPVHGSCRNITCDSWFTSIPLAKNLLQEPYKLTIVGTVRSNAREIPEVLKNSRSAPVGTSMFCFDGPLTLVSYKPKPAKMVYLLSSCDEDASINESTGKPQMVMYYNQTKGGVDTLNQMCSVMTCSRKTNRAPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPSKIRRKASAACKKCKKVICREHNIDVCQSCL

SEQ ID NO: 7 - protein, 594 aa, Artificial Sequence
  Modified hyperactive PiggyBac
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVAKIWDLFIHQCIQNYTPGAHLTIDEQLLGFAGACPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLDQMCSVMTCSRKTNRAPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 8 - protein, 594 aa, Artificial Sequence
  Modified hyperactive PiggyBac
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSAPVGTSMFCFDGPLTLVSYKPKPAKMVYLLSSCDEDASINESTGKPQMVMYYNQTKGGVDTLNQMCSVMTCSRKTNRAPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASAACKKCKKVICREHNIDVCQRCL

SEQ ID NO: 9 - protein, 594 aa, Artificial Sequence
  Hyperactive PiggyBac aa sequence
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 10 - protein, 594 aa, Artificial Sequence
  Modified hyperactive PiggyBac
  SITE 245..245: Wherein amino acid can be Arg, Ala
  SITE 268..268: Wherein amino acid can be Asp, Asn
  SITE 275..275: Wherein amino acid can be Arg, Ala
  SITE 277..277: Wherein amino acid can be Arg, Ala
  SITE 287..287: Wherein amino acid can be Ala, Lys
  SITE 290..290: Wherein amino acid can be Ala, Lys
  SITE 315..315: Wherein amino acid can be Arg, Ala
  SITE 325..325: Wherein amino acid can be Gly, Ala
  SITE 341..341: Wherein amino acid can be Arg, Ala
  SITE 346..346: Wherein amino acid can be Asp, Asn
  SITE 347..347: Wherein amino acid can be Asn, Ala, Ser
  SITE 350..350: Wherein amino acid can be Thr, Ala
  SITE 351..351: Xaa can be Ser, Glu, Pro, Ala
  SITE 356..356: Wherein amino acid can be Lys, Glu
  SITE 357..357: Wherein amino acid can be Asn, Ala
  SITE 372..372: Wherein amino acid can be Arg, Ala
  SITE 375..375: Wherein amino acid can be Lys, Ala
  SITE 388..388: Wherein amino acid can be Arg, Ala
  SITE 409..409: Wherein amino acid can be Lys, Ala
  SITE 412..412: Wherein amino acid can be Lys, Ala
  SITE 432..432: Wherein amino acid can be Lys, Ala
  SITE 460..460: Wherein amino acid can be Arg, Ala
  SITE 461..461: Wherein amino acid can be Ala, Lys
  SITE 465..465: Wherein amino acid can be Trp, Ala
  SITE 560..560: Wherein amino acid can be Thr, Ala
  SITE 564..564: Wherein amino acid can be Ser, Pro
  SITE 571..571: Wherein amino acid can be Asn, Ser
  SITE 573..573: Wherein amino acid can be Ser, Ala
  SITE 576..576: Wherein amino acid can be Lys, Ala
  SITE 586..586: Wherein amino acid can be His, any naturally occuring amino acid
  SITE 587..587: Wherein amino acid can be Ile, Ala
  SITE 589..589: Wherein amino acid can be Met, Val
  SITE 592..592: Wherein amino acid can be Ser, Gly
  SITE 594..594: Wherein amino acid can be Phe, Leu
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVXKIWDLFIHQCIQNYTPGAHLTIXEQLLGFXGXCPFRVYIPNXPSXYGIKILMMCDSGTKYMINGMPYLGXGTQTNGVPLXEYYVKELSKPVHGSCXNITCXXWFXXIPLAXXLLQEPYKLTIVGTVXSNXREIPEVLKNSRSXPVGTSMFCFDGPLTLVSYKPXPAXMVYLLSSCDEDASINESTGXPQMVMYYNQTKGGVDTLDQMCSVMTCSXXTNRXPMALLYGMINIACINSFIIYSHNVSSKGEKVQSRKKFMRNLYMGLTSSFMRKRLEAPTLKRYLRDNISNILPKEVPGTSDDSTEEPVMKKRTYCXYCPXKIRRKAXAXCKXCKKVICREHXXDXCQXCX

SEQ ID NO: 11 - protein, 594 aa, Artificial Sequence
  Modified hyperactive PiggyBac with mutations in the catalytic triad
  SITE 268..268: Xaa can be Asp, Asn
  SITE 346..346: Xaa can be Asp, Asn
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIXEQLLGFRGRCPFRVYIPNKPSKYGIKILMMCDSGTKYMINGMPYLGRGTQTNGVPLGEYYVKELSKPVHGSCRNITCXNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 12 - protein, 594 aa, Artificial Sequence
  Modified hyperactive PiggyBac with mutations in amino acids that are critical for excision
  SITE 287..287: Xaa can be Lys, Ala
  SITE 290..290: Xaa can be Lys, Ala
  SITE 460..460: Xaa can be Arg, Ala
  SITE 461..461: Xaa can be Lys, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNXPSXYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSXXTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 13 - protein, 594 aa, Artificial Sequence
  Modified hyperactive PiggyBac with mutation that are involved in target joining
  SITE 351..351: Xaa can be Ser, Glu, Pro, Ala
  SITE 356..356: Xaa can be Lys, Glu
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTXIPLAXNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 14 - protein, 594 aa, Artificial Sequence
  Modified hyperactive PiggyBac with mutations that are critical for integration
  SITE 560..560: Wherein amino acid can be Thr, Ala,
  SITE 564..564: Wherein amino acid can be Ser, Pro
  SITE 571..571: Wherein amino acid can be Asn, Ser
  SITE 573..573: Wherein amino acid can be Ser, Ala
  SITE 589..589: Wherein amino acid can be Met, Val
  SITE 592..592: Wherein amino acid can be Ser, Gly
  SITE 594..594: Wherein amino acid can be Phe, Leu
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCXYCPXKIRRKAXAXCKKCKKVICREHNIDXCQXCX

SEQ ID NO: 15 - protein, 594 aa, Artificial Sequence
  Modified hyperactive PiggyBac with mutations that are involved in alignment
  SITE 325..325: Xaa can be Gly, Ala
  SITE 347..347: Xaa can be Asn, Ala, Ser
  SITE 350..350: Xaa can be Thr, Ala
  SITE 465..465: Xaa can be Trp, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLXEYYVKELSKPVHGSCRNITCDXWFXSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRXPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 16 - protein, 594 aa, Artificial Sequence
  Modified hyperactive PiggyBac with mutations at well conserved amino acids
  SITE 576..576: Xaa can be lys, Ala
  SITE 587..587: Xaa can be Ile, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKXCKKVICREHNXDMCQSCF

SEQ ID NO: 17 - protein, 594 aa, Artificial Sequence
  Modified hyperactive PiggyBac with mutations involved in Zn2+ binding
  SITE 586..586: Wherein amino acid can be His, any naturally occuring amino acid
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHXIDMCQSCF

SEQ ID NO: 18 - protein, 594 aa, Artificial Sequence
  Modified hyperactive PiggyBac with mutations that are involved in integration
  SITE 315..315: Wherein amino acid can be Arg, Ala
  SITE 341..341: Wherein amino acid can be Arg, Ala
  SITE 372..372: Wherein amino acid can be Arg, Ala
  SITE 375..375: Wherein amino acid can be Lys, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGXGTQTNGVPLGEYYVKELSKPVHGSCXNITCDNWFTSIPLAKNLLQEPYKLTIVGTVXSNXREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 19 - protein, 897 aa, Artificial Sequence
  Cas9 from Corynebacterium ulcerans
MTNAVANHHVLWAKFDNVSEPYPLLAHLLDTATAATCLFNHWLRKGLRDRLSTELGPDAEKILGFVAGIHDLGKANPYFQAQRRNKKEEWITLRDAIQKAGFPLSNGTSALFEETKEKRRHENITLSILGWEITKFLQVKDVWPQLAIIGHHGNFSAPGFLSDEDDLEDIEDIFDDNGWSPTHELLVSSLLQAVGLEKQPEIKHISPASAILISGLVVLADRIASQSEMASDGLQALQKEELFFHQPEKWIANRKAFCREIIENTVGTYHPWESEAAGIRAVLGDYEPRFTQKAALNAGDGLFNVMETTGAGKTEAALLRHVKRKERLLFFLPTQATTNAIMDRIGKIFDGTPNVASLAHGLAVTEDFYAHPILPVQGSSDDANYKDNGGLYPTEFVRSAGTPRLLAPVCVGTIDQALMGALPSKFNHLRLLALANAHVVVDEVHTMDQYQSELMSGLLEWWSATDTPVTLLTATMPAWQREKFHLSYTGKDPHFKGVFPSLEDWSTPSKNTETSQENIPTEAFTIPINIDKIAHNEIVDSHVQWVIEQRKLFPQARIGIICNTVGRAQSIAEALAHESPIVLHSRMTAGHRKEAATKLEQAIGKKGTANATLVIGTQAIEASLDIDLDLLRTELCPAPSLIQRAGRLWRRLDPQREVRVPGMVGKKLTIAVVDSPSTGQTLPYLRSQLYRVESWLKQRDRIEFPADIQDFIDATTPGLQELFQKVSLPEDCGSAEEREALADDYLNEVASWVTKQRQAGTSRIDFAKHGKPRQVLASDCVVEDFLQITSANNLEESATRLIDYPSISAILCDPTGTIPGAWTDSVEKLIAISAKDSESLRRALRASISIPHSKKFLPITSREIPLSEAKTLLSGYSAVHIQPDEYDLQSGLKGPQK

SEQ ID NO: 20 - protein, 876 aa, Artificial Sequence
  Cas9 from Corynebacterium diphtheria
MNPHEELWAKQKGLAKPYPLLAHLLDSAAVAGALWDHWLRQDLRQMFIEELGSNAREIIQFVVGSHDIGKATPLFQYQKAQKGEVWDSIRYAIDRTGRYQKPLPSSYLVKKTSGGPNRHEQWSSFASKNEYLKPSAAAKENWIGLAIGGHHGRFEPVGYGRHQRKAAEDLAKSGWSAAQQDLLRALEKASGITRASLPSELSPELTLVLSGLTILADRISSTESFVITGARMIDDGTLHLATPIDWLKTRKLDSEKHVAKTVGIYHGWNNHESAIHSILKGYDPRPLQTIALQNQVGLLNLMAPTGNGKTEAAILRHSLKENDRLIFLLPTQATSNAIMRRVQGIYSDTPNAAALAHSLASVEDFYQTPLSVFDDHYDPSKEQFESSMSGGLYPSSFVCSGAARLLAPICIGTVDQALATALPGKWIHLRILALANAHIVIDEVHTLDHYQTALLENILPILAKLKTKITFLTATMPSWQRTKLLTAYGGEDLQIPPTVFPAAETVLPGQFNRTLIDSDSTTIDFTMEETSYDHLVESHVKWHQTTRLNAPHARIGLICNTVKRAQEIAAALEKTNDRIVLLHSRMTTEHRRRSAELLESLLGPNGNRKTITVVGTQAIEASLDIDLDILRTELCPAPSLVQRAGRVWRRNDPYRSSRITADHKPISVVFIAEAKDWQVLPYLRAETSRTQRWLEKHNQMFLPQMAQEFIDAATVDLDTATSEMDLDALALMGIHLMKADGAKARIQDVLNSDSKVSDFALLTSKNEIDEAQTRLIEEGTHLRIILGDENESIPGGWKHGLSSLLKLKASDRESLRTALLASIPLLVSEKQKQLLYQHNLVPLSSSKTVLAGFYFLPKAQNFYSKNLGFIWPEEKD

SEQ ID NO: 21 - protein, 773 aa, Artificial Sequence
  Cas9 from Spiroplasma syrphidicola
MNYKKLILGLDLGIASCGWAVTGQMEDGNWVLDDFGVRLFQTPENSKDGTTNAAARRLKRGARRLIKRRKNRIKDLKNLFEKINFINKASLDKYINEHSATNLVEDFNRHELYNPYFLRSIGITEKLTREELVWSLIHIANRRGYKNKFAFDIEGDGKKRETKLDEAISNALISSNLTISQEIVRNKKFRDAKNKKALLVRNKGGKEGENNFQFLFARDDYKKEVDLLLAKQAKFYPELTEEIRAKAADIIFRQRDFEDGPGPKKQELREIYKKENKQFSKNFTQLEGRCTFLRELSVGYKSSILFDLFHIISEVSKISKYIEENDQLAQDIISSFLYNEAGKKGKTLLKEILKKHHINDDIFDTNAYKNIDFKTNYLNLLKEVFGNDVLKNLSLNRLEDNIYHQLGFIIHTNITPERKEKAINQWLLENNIILAKEKLNILLKPNSSISTTVKTSFKWMSIAISNFLKGIPYGKFQAQFIKEDNFKLPESYAKQYQKYLTGEKTFEMFAPIIDPDLWRNPIVFRAINQARKVIKKLFEKYTFIDQINIELTREMGLSFSDRKKVKERQDDSLKENAKAKEFLMANGIIVNDTNVLKYKLWIQQNKKSLYSGKEITIADLGASNVLQIDHIIPYSKLADDSFNNKVLVFSKENQEKGNQFADQYVKSLGTENYNNYKKRVNYLLFQNQINQKKAEYLLCSNQNEEILNDFVSRNLNDTRYITRYVTNWLKAEFELQSRFGLAKPKIMTLNGAITSRFRRTWLRNSPWGLEKKS

SEQ ID NO: 22 - protein, 1380 aa, Artificial Sequence
  Cas9 from Prevotella intermedia
MKRILGLDLGTTSIGWALVNEAENNNEASSIVRLGVRVNPLTVDEKSNFEKGKAITTNADRQLRHGARINLQRYKLRRQNLHDCLQKQGWLGTEAMYEEGKASTFETYKLRAKAAEEEISLHEFARVLFMLNKKRGYKSNRKANNKEDGQLFDGMTIAKKLYEEHLTPAEYSLQLLNKGKKFTQGYYRSDLNAELERIWDEQKKYYPEILTDEFKQQLEGKTKTNTSKIFLAKYGIYSADLKGLDRKFQPLKWRVEALQQQVDKEVLAFVISDLKGQIANTSGLLGAISDRSKELYFNKQTVGQYLWASLEENPHISIKNKPFYRQDYLDEFEKIWETQAAFHKQLTPELKQEIRDIIIFYQRPLKSKKSLISVCELEQRKVKATIDGKEKEITIGPKVAPKSSPVFQEFRIWQNLNNVLLIDNDTNEKRPLDEVERNLLYKELSIKAKLSKTEALKILNKKGKQWDLNYRELEGNRTQAILFDCYNRIITLTGHEECDFKKIKASEIRHYVSTIFKNLGFSTEILDFDPSLKKHELEKQPMYQLWHLLYSYESDNSRTGNESLLRKLETTFGFPEEYATVLCDVVFEEDYGNLSVKAMREILPYLQAGNDYSQACAYAGYNHSRHSLTKEELDQKVYKERLELLPKNSLRNPVVEKILNQMINVINAIIDEYGKPDEIRIEMARELKSSAADRKKTTHAISQGNAENQRIREILEKEFSLSYISRNDIIKYKLYEELEPNYYKTLYSDTYITKDKLFSKDFDIEHIIPKARLFDDSFSNKTLEARNINLEKSNKTAFDFIKEKYGEDGAEAYKKKLDMLLENDAISRPKYNNLLRAEADIPSDFINRDLRNTQYIAKKACEILGELVKTVTPTTGKITNRLREDWQLVDVMKELNFEKYEKLGLTEIVEDRDGRKIKRIKDWTKRNDHRHHAMDALAIAFTKPSFIQYLNNLNARSNKGDSIYAIENKELHYEEGKLRFNAPIPVNEFRAEAKRHLSAILVSIKAKNKVMTQNVNKIKTKHGIIKKIQLTPRGPLHNETIYGTKMRPIIKMVKVGAALDEATINKVSSPAIREALLKRLNEYSGNAKKAFTGKNTLEKNPIYLNAGRTKTVPSLVKTVEWESFHPTRKLIDKDLNVDKVVDKGIREILKARLEEFNGDAKKAFSNLEENPIYLDEAKKIALKRVSIEGVLSAIPLHTLKNQAGKPITGKDGKPVLGNYVQTSNNHHIAFYYDEDGNLQDNAVSFFEAAERKSQGIPVIDKDYNRDKGWRFLFTMKQNEYFVFPNEATGFIPSEVDLTDEANYGIISPNLYRVQKVSRIDKGTSASRDYWFRHHLETILNDDAKLKNLAFKRIRGLLELKDIIKVRINSTGKIVAVGEYD

SEQ ID NO: 23 - protein, 535 aa, Artificial Sequence
  Cas9 from Spiroplasma taiwanense
MWSRKILKAGSRLFDEANLSDKIASKRREQRGRRRNLRRKITWKQDLINLFVKYNFLQKENDFYELDFNFDLLELRKKAINSKIELEQLLIILFNYIKHRGSFNYREDLSELKNISQEELETSSEFKLPVDIQFELKEENNKFREINNEKSLINHEWYVKEINLILDAQIENKLINLDFKKDYLKLFNRKREYYDGPGPKDKNLLNPSKYGWKNQEEFFDRFAGKDTYDSKEQRAPKHSLTSYLFNILNDLNNLSINGDRNQLTYENKKDLINLTLINQKEKAENITLKKIAKYLKINEKNITGYRLKPNSNESIFTVFESANKMRSILVKNNKSIDFICLENIDKIDKIVDILTKYQSIEDKSLKLEELNFDFFDKETCEKLAVISLTGTHALSKKTMSKLIEEMFHDNLNHMEALAKLKIKPDYKLKVDLTNFKTIPILREKINEMYISPVVKRALIESLKIIKELERHFKDFEIKDIVIEMAKKNSAEKKQFISKIQRQNVDLVKKLSNDYSLDENKLNFKMKEKFLLLSEQ

SEQ ID NO: 24 - protein, 1281 aa, Artificial Sequence
  Cas9 from Streptococcus iniae
MRKPYSIGLDIGTNSVGWAVITDDYKVPSKKMRIQGTTDRTSIKKNLIGALLFDNGETAEATRLKRTTRRRYTRRKYRIKELQKIFSSEMNELDIAFFPRLSESFLVSDDKEFENHPIFGNLKDEITYHNDYPTIYHLRQTLADSDQKADLRLIYLALAHIIKFRGHFLIEGNLDSENTDVHVLFLNLVNIYNNLFEEDIVETASIDAEKILTSKTSKSRRLENLIAEIPNQKRNMLFGNLVSLALGLTPNFKTNFELLEDAKLQISKDSYEEDLDNLLAQIGDQYADLFIAAKKLSDAILLSDIITVKGASTKAPLSASMVQRYEEHQQDLALLKNLVKKQIPEKYKEIFDNKEKNGYAGYIDGKTSQEEFYKYIKPILLKLDGTEKLISKLEREDFLRKQRTFDNGSIPHQIHLNELKAIIRRQEKFYPFLKENQKKIEKLFTFKIPYYVGPLANGQSSFAWLKRQSNESITPWNFEEVVDQEASARAFIERMTNFDTYLPEEKVLPKHSPLYEMFMVYNELTKVKYQTEGMKRPVFLSSEDKEEIVNLLFKKERKVTVKQLKEEYFSKMKCFHTVTILGVEDRFNASLGTYHDLLKIFKDKAFLDDEANQDILEEIVWTLTLFEDQAMIERRLVKYADVFEKSVLKKLKKRHYTGWGRLSQKLINGIKDKQTGKTILGFLKDDGVANRNFMQLINDSSLDFAKIIKNEQEKTIKNESLEETIANLAGSPAIKKGILQSIKIVDEIVKIMGQNPDNIVIEMARENQSTMQGIKNSRQRLRKLEEVHKNTGSKILKEYNVSNTQLQSDRLYLYLLQDGKDMYTGKELDYDNLSQYDIDHIIPQSFIKDNSIDNTVLTTQASNRGKSDNVPNIETVNKMKSFWYKQLKSGAISQRKFDHLTKAERGALSDFDKAGFIKRQLVETRQITKHVAQILDSRFNSNLTEDSKSNRNVKIITLKSKMVSDFRKDFGFYKLREVNDYHHAQDAYLNAVVGTALLKKYPKLEAEFVYGDYKHYDLAKLMIQPDSSLGKATTRMFFYSNLMNFFKKEIKLADDTIFTRPQIEVNTETGEIVWDKVKDMQTIRKVMSYPQVNIVMKTEVQTGGFSKESIWPKGDSDKLIARKKSWDPKKYGGFDSPIIAYSVLVVAKIAKGKTQKLKTIKELVGIKIMEQDEFEKDPIAFLEKKGYQDIQTSSIIKLPKYSLFELENGRKRLLASAKELQKGNELALPNKYVKFLYLASHYTKFTGKEEDREKKRSYVESHLYYFDVRLSQVFRVTNVEF

SEQ ID NO: 25 - protein, 1352 aa, Artificial Sequence
  Cas9 from Belliella baltica
MKKILGLDLGTTSIGWAFIKEPEKDVVGSEIVDMGVRIVPLSSDEENDFAKGNTISINADRTLKRGARRNLQRFKQRRNALLEIFKEKKLISTNFKYAEDGPSSTFSTLNLRAKAAKEKIELQDLVKVLLQINKKRGYKSSRKAKSEEDDGSAIDSMGIAKELYENDLTPGQWVYEALQKGRKNVPDFYRSDLQEEFKKIVNYQSEFFPDIFNASFVEDWMGKASTPTKQYFNKKGVQLAENKGKREERRLQEYKWRAEAVNFKIDLSEIALILSQINSQISNSSGYLGAISDRSKELYFKNLTVGQYLYQQIKKNPHTRLKGQVFYRQDYLDEFERIWSVQSSFYPQLNDALKREVRDITIFFQRRLKSQKHLISNCEFEDHHKVVPKSHPVFQEFRIWQNLNNLLLIKKDNLNEKFDLELESKIALANELAFKRELNVKDALKILGLKPNEWEFNFTKIEGNRTNQAFFDAFAKIIELEDGEPIDLGDLKADDILDQFSEAFLRIGIDTELLQVNSDIEGAEYEKQSYIQFWHLLYSSEDDQKLKLNLIRKFGFKPEHAKILASISLQDDHASLSSRAIKKILPHLQSGLIYDKACTYAGYNHSSSFTKDENEKRELRAELELLKKNSLRNPVVEKILNQMINVVNAILKDPELGRPDEIRVEMARELKANAEQRKNMTSNIASATRDHDKYREILKSEFGLKRVTKNDLLRYKLWLETDGISLYTGKPIEASKLFSKEYDIEHIIPKARLFDDSFSNKTICERQLNIDKANVTAFSFLQNKLSADEFEQYQSRVKSLYGKLSKAKIQKLLMANDKIPEDFIARQLQETRYISKKAKEILFEISRRVSVTTGTITDKLREDWGLVEIMKELNWEKYDKLGLTYTIEGKHGERLNKIKDWSKRNDHRHHAMDALTVALTKPAYIQYLNNLNAKGLNNKKGTEVFAIEQKYLKRENGKLCFIPPIENIRSEAKKHLSRILVSYKAKNKVVTINKNKTKSKAGLNEQIALTPRGQLHKETVYGKSFHYSTKFEKIGASFNVQKINTVAKKEEREALLKRLAENGNDPKKAFTGKNTLNKMPIYLDLGKNIKLSEKVKTVVLEQNYTIRKNIDPDLKVDKVIDVGIKRILESRLEEFGGNAKLAFSNLEENPIWLNKEKGISIKRVKISGVSNVESLHVKKDHFGEPILDQEGNEIPVDFVSTGNNHHVAIYEDENGNLQEEVVSFFEAVVRQNQGLPIIKKNHTLGWKFLFTLKQNEYFVFPSDDFVPADVDLMDEQNYHLISPNLFRVQKIARKNYVFNNHLETKAVDNDLLKSKKELSKITYHFYQTPEHLRGIIKIRINHLGKIIQIGEY

SEQ ID NO: 26 - protein, 1509 aa, Artificial Sequence
  Cas9 from Psychroflexus torquisI
MKRILGLDLGTNSIGWSLIEHDFKNKQGQIEGLGVRIIPMSQEILGKFDAGQSISQTADRTKYRGVRRLYQRDNLRRERLHRVLKILDFLPKHYSESIDFQDKVGQFKPKQEVKLNYRKNEKNKHEFVFMNSFIEMVSEFKNAQPELFYNKGNGEETKIPYDWTLYYLRKKALTQQITKEELAWLILNFNQKRGYYQLRGEDIDEDKNKKYMQLKVNNLIDSGAKVKGKVLYNVIFDNGWKYEKQIVNKDEWEGRTKEFIITTKTLKNGNIKRTYKAVDSEIDWAAIKAKTEQDINKANKTVGEYIYESLLDNPSQKIRGKLVKTIERKFYKEEFEKLLSKQIELQPELFNESLYKACIKELYPRNENHQSNNKKQGFEYLFTEDIIFYQRPLKSQKSNISGCQFEHKIYKQKNKKTGKLELIKEPIKTISRSHPLFQEFRIWQWLQNLKIYNKEKIENGKLEDVTTQLLPNNEAYVTLFDFLNTKKELEQKQFIEYFVKKKLIDKKEKEHFRWNFVEDKKYPFSETRAQFLSRLAKVKGIKNTEDFLNKNTQVGSKENSPFIKRIEQLWHIIYSVSDLKEYEKALEKFAEKHNLEKDSFLKNFKKFPPFVSDYASYSKKAISKLLPIMRMGKYWSESAVPTQVKERSLSIMERVKVLPLKEGYSDKDLADLLSRVSDDDIPKQLIKSFISFKDKNPLKGLNTYQANYLVYGRHSETGDIQHWKTPEDIDRYLNNFKQHSLRNPIVEQVVMETLRVVRDIWEHYGNNEKDFFKEIHVELGREMKSPAGKREKLSQRNTENENTNHRIREVLKELMNDASVEGGVRDYSPSQQEILKLYEEGIYQNPNTNYLKVDEDEILKIRKKNNPTQKEIQRYKLWLEQGYISPYTGKIIPLTKLFTHEYQIEHIIPQSRYYDNSLGNKIICESEVNEDKDNKTAYEYLKVEKGSIVFGHKLLNLDEYEAHVNKYFKKNKTKLKNLLSEDIPEGFINRQLNDSRYISKLVKGLLSNIVRENGEQEATSKNLIPVTGVVTSKLKQDWGLNDKWNEIIAPRFKRLNKLTNSNDFGFWDNDINAFRIQVPDSLIKGFSKKRIDHRHHALDALVVACTSRNHTHYLSALNAENKNYSLRDKLVIKNENGDYTKTFQIPWQGFTIEAKNNLEKTVVSFKKNLRVINKTNNKFWSYKDENGNLNLGKDGKPKKKLRKQTKGYNWAIRKPLHKETVSGIYNINAPKNKIATSVRTLLTEIKNEKHLAKITDLRIRETILPNHLKHYLNNKGEANFSEAFSQGGIEDLNKKITTLNEGKKHQPIYRVKIFEVGSKFSISEDENSAKSKKYVEAAKGTNLFFAIYLDEENKKRNYETIPLNEVITHQKQVAGFPKSERLSVQPDSQKGTFLFTLSPNDLVYVPNNEELENRDLFNLGNLNVEQISRIYKFTDSSDKTCNFIPFQVSKLIFNLKKKEQKKLDVDFIIQNEFGLGSPQSKNQKSIDDVMIKEKCIKLKIDRLGNISKA

SEQ ID NO: 27 - protein, 1388 aa, Artificial Sequence
  Cas9 from Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 28 - protein, 1334 aa, Artificial Sequence
  Cas9 from Listeria innocua
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 29 - protein, 984 aa, Artificial Sequence
  Cas9 from Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 30 - protein, 1082 aa, Artificial Sequence
  Cas9 from Neisseria. meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 31 - protein, 1367 aa, Artificial Sequence
  Cas9 from Streptococcus pyogenes
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 32 - DNA, 528 bp, Artificial Sequence
  zfp coding sequence
atggcccaggctgctcttgagcccggagagaaaccctacaagtgcccggagtgcggaaagtccttctctgagcggagtcacctccgagagcaccagcggactcatacgggcgaaaaaccatacaagtgcccagaatgtggtaaatctttttctcgggctgacaacctgactgaacatcagcgcacgcacaccggtgaaaaaccttacaagtgtccagagtgtggcaagagcttttctagtagaaggacctgtcgagcgcatcagcggactcacaccggcgaaaaaccctataagtgtccggaatgtggaaagagctttagccgcaacgacacccttactgaacaccagcgaacacacacgggagaaaaaccatataaatgtccggaatgtggcaaaagttttagtcggagtgataaacttacggagcaccaacggacacacaccggagagaagccatataagtgtcctgaatgtggaaagtccttctcacagcttgctcatctgcgagcacatcagcgcacacacacc

SEQ ID NO: 33 - protein, 176 aa, Artificial Sequence
  ZFP amino acid sequence
MAQAALEPGEKPYKCPECGKSFSERSHLREHQRTHTGEKPYKCPECGKSFSRADNLTEHQRTHTGEKPYKCPECGKSFSSRRTCRAHQRTHTGEKPYKCPECGKSFSRNDTLTEHQRTHTGEKPYKCPECGKSFSRSDKLTEHQRTHTGEKPYKCPECGKSFSQLAHLRAHQRTHT

SEQ ID NO: 34 - DNA, 555 bp, Artificial Sequence
  ZNF-E2C Nucleotide
atggcgcaggcggcgctggaaccgggcgaaaaaccgtataaatgcccggaatgcggcaaaagctttagccgcaaagatagcctggtgcgccatcagcgcacccataccggcgaaaaaccgtataaatgcccggaatgcggcaaaagctttagccagagcggcgatctgcgccgccatcagcgcacccataccggcgaaaaaccgtataaatgcccggaatgcggcaaaagctttagcgattgccgcgatctggcgcgccatcagcgcacccataccggcgaaaaaccgtataaatgcccggaatgcggcaaaagctttagccagagcagccatctggtgcgccatcagcgcacccataccggcgaaaaaccgtataaatgcccggaatgcggcaaaagctttagcgattgccgcgatctggcgcgccatcagcgcacccataccggcgaaaaaccgtataaatgcccggaatgcggcaaaagctttagccgcagcgataaactggtgcgccatcagcgcacccataccggcaaaaaaaccagcggccaggcgggc

SEQ ID NO: 35 - protein, 185 aa, Artificial Sequence
  ZNF-E2C Aminoacid
MAQAALEPGEKPYKCPECGKSFSRKDSLVRHQRTHTGEKPYKCPECGKSFSQSGDLRRHQRTHTGEKPYKCPECGKSFSDCRDLARHQRTHTGEKPYKCPECGKSFSQSSHLVRHQRTHTGEKPYKCPECGKSFSDCRDLARHQRTHTGEKPYKCPECGKSFSRSDKLVRHQRTHTGKKTSGQAG

SEQ ID NO: 36 - DNA, 555 bp, Artificial Sequence
  ZNF-E3 Nucleotide
atggcgcaggcggcgctggaaccgggcgaaaaaccgtataaatgcccggaatgcggcaaaagctttagcgatccgggcgcgctggtgcgccatcagcgcacccataccggcgaaaaaccgtataaatgcccggaatgcggcaaaagctttagccagagcagccatctggtgcgccatcagcgcacccataccggcgaaaaaccgtataaatgcccggaatgcggcaaaagctttagcgattgccgcgatctggcgcgccatcagcgcacccataccggcgaaaaaccgtataaatgcccggaatgcggcaaaagctttagccagagcagccatctggtgcgccatcagcgcacccataccggcgaaaaaccgtataaatgcccggaatgcggcaaaagctttagcgattgccgcgatctggcgcgccatcagcgcacccataccggcgaaaaaccgtataaatgcccggaatgcggcaaaagctttagccagagcagccatctggtgcgccatcagcgcacccataccggcaaaaaaaccagcggccaggcgggc

SEQ ID NO: 37 - protein, 185 aa, Artificial Sequence
  ZNF-E3 Amino acid
MAQAALEPGEKPYKCPECGKSFSDPGALVRHQRTHTGEKPYKCPECGKSFSQSSHLVRHQRTHTGEKPYKCPECGKSFSDCRDLARHQRTHTGEKPYKCPECGKSFSQSSHLVRHQRTHTGEKPYKCPECGKSFSDCRDLARHQRTHTGEKPYKCPECGKSFSQSSHLVRHQRTHTGKKTSGQAG

SEQ ID NO: 38 - DNA, 528 bp, Artificial Sequence
  ZNF-TRCa Nucleotide
atggcgcaggcggctcttgaacccggggagaaaccctataaatgccctgagtgtggcaagagtttttcaaccacaggaaacttgacagtccaccaacggacccacaccggcgagaaaccatacaagtgtccggagtgtggtaagtctttctcaagtcctgccgaccttaccagacatcaacgcacacatacaggtgaaaaaccttacaagtgcccagagtgcggaaaaagtttttcacaatctggcgacctccgcaggcaccagcgcactcacaccggtgaaaaaccatacaagtgtcctgagtgcgggaagagttttagtcaacgagctcatctggagcgacaccaaaggactcatactggggagaaaccgtacaaatgtcccgaatgtgggaagagcttctctaccaagaattcccttacagagcaccagcgcacgcatacgggagagaagccgtataagtgtccggaatgtggcaagagcttttccagaagtgaccaccttacaacccaccagaggacgcacacc

SEQ ID NO: 39 - protein, 176 aa, Artificial Sequence
  ZNF-TRCa Aminoacid
MAQAALEPGEKPYKCPECGKSFSTTGNLTVHQRTHTGEKPYKCPECGKSFSSPADLTRHQRTHTGEKPYKCPECGKSFSQSGDLRRHQRTHTGEKPYKCPECGKSFSQRAHLERHQRTHTGEKPYKCPECGKSFSTKNSLTEHQRTHTGEKPYKCPECGKSFSRSDHLTTHQRTHT

SEQ ID NO: 40 - DNA, 18 bp, Artificial sequence
  AAVS1 site
agacggccgcgtcagagc

SEQ ID NO: 41 - protein, 7 aa, Artificial Sequence
  Zinc Finger 1
ERSHLRE

SEQ ID NO: 42 - protein, 7 aa, Artificial Sequence
  Zinc Finger 2
RADNLTE

SEQ ID NO: 43 - protein, 7 aa, Artificial Sequence
  Zinc Finger 3
SRRTCRA

SEQ ID NO: 44 - protein, 7 aa, Artificial Sequence
  Zinc Finger 4
RNDTLTE

SEQ ID NO: 45 - protein, 7 aa, Artificial Sequence
  Zinc Finger 5
RSDKLTE

SEQ ID NO: 46 - protein, 7 aa, Artificial Sequence
  Zinc Finger 6
QLAHLRA

SEQ ID NO: 47 - DNA, 51 bp, Artificial Sequence
  Nuclear Localization Signal
atggctccaaagaaaaagaggaaagtgggaatccacggagtccccgccgct

SEQ ID NO: 48 - DNA, 30 bp, Artificial Sequence
  GGS linker Nucleic Acid
ggtggatctggcggtggatctggtggcggt

SEQ ID NO: 49 - protein, 10 aa, Artificial Sequence
  GGS linker Amino Acid
GGSGGGSGGG

SEQ ID NO: 50 - DNA, 36 bp, Artificial Sequence
  GGS4x Linker Nucleic Acid
ggagggagtggtgggtccggtggtagtggcggatcc

SEQ ID NO: 51 - protein, 12 aa, Artificial Sequence
  GGS4x Linker Amino Acid
GGSGGSGGSGGS

SEQ ID NO: 52 - DNA, 45 bp, Artificial Sequence
  GGS5x Linker Nucleic Acid
ggaggctccggtgggtctggtgggagcggtggtagtggcggatcc

SEQ ID NO: 53 - protein, 15 aa, Artificial Sequence
  GGS5x Linker Amino Acid
GGSGGSGGSGGSGGS

SEQ ID NO: 54 - DNA, 54 bp, Artificial Sequence
  GGS6x Linker Nucleic Acid
ggaggcagtggtgggagcggtggttccgggggtagtggtggttccgggggatcc

SEQ ID NO: 55 - protein, 18 aa, Artificial Sequence
  GGS6x Linker Amino Acid
GGSGGSGGSGGSGGSGGS

SEQ ID NO: 56 - DNA, 63 bp, Artificial Sequence
  GGS7x Linker Nucleic Acid
ggaggttctggaggctccggtgggtccgggggaagtggggggtcaggcggatcaggaggatcc

SEQ ID NO: 57 - protein, 21 aa, Artificial Sequence
  GGS7x Linker Amino Acid
GGSGGSGGSGGSGGSGGSGGS

SEQ ID NO: 58 - DNA, 75 bp, Artificial Sequence
  GGS8x Linker Nucleic Acid
ggaggtagcggaggttccggagggagcggcgggagtgggggaagcgggggaagtggaggatccgggggaggatcc

SEQ ID NO: 59 - protein, 21 aa, Artificial Sequence
  GGS8x Linker Amino Acid
GGSGGSGGSGGSGGSGGSGGS

SEQ ID NO: 60 - DNA, 48 bp, Artificial Sequence
  Linker XTEN Nucleic Acid
tccggtagcgaaacaccggggacttcagaatcggccaccccggagtct

SEQ ID NO: 61 - protein, 16 aa, Artificial Sequence
  Linker XTEN Amino Acid
SGSETPGTSESATPES

SEQ ID NO: 62 - DNA, 36 bp, Artificial Sequence
  Linker B Nucleic Acid
ggaagcgccggtagtgcggctgggtctggcgagttc

SEQ ID NO: 63 - protein, 12 aa, Artificial Sequence
  Linker B Amino Acid
GSAGSAAGSGEF

SEQ ID NO: 64 - DNA, 4104 bp, Artificial Sequence
  human Cas9 (hCas9) nucleic acid
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 65 - DNA, 4104 bp, Artificial Sequence
  nickase Cas9 (nCas9) nucleic acid
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaacaggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 66 - DNA, 4104 bp, Artificial Sequence
  dead Cas9 (dCas9) nucleic acid
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 67 - DNA, 1782 bp, Artificial Sequence
  hyperactive PiggyBac (PB) transposase nucleic acid
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 68 - DNA, 1020 bp, Artificial Sequence
  hyperactive Sleeping Beauty (SB100) transposase nucleic acid
atgggaaaatcaaaagaaatcagccaagacctcagaaaaagaattgtagacctccacaagtctggttcatccttgggagcaatttccaaacgcctggcggtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccactcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatggacgccgtgcagtatgtggatatattgaagcaacatctcaagacatcagtcaggaagttaaagcttggtcgcaaatgggtcttccaacacgacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaaattattgtgggaagcttgtggaaggctacccgaaacgtttgacccaagttaaacaatttaaaggcaatgctaccaaatac

SEQ ID NO: 69 - protein, 1368 aa, Artificial Sequence [sequence omitted]

SEQ ID NO: 70 - protein, 1368 aa, Artificial Sequence
  nickase Cas9 (nCas9) amino acid
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 71 - protein, 1368 aa, Artificial Sequence
  dead Cas9 (dCas9) amino acid
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 72 - protein, 1069 aa, Staphylococcus aureus
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 73 - protein, 340 aa, Artificial Sequence
  hyperactive Sleeping Beauty (SB100) transposase amino acid
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 74 - protein, 1236 aa, Artificial Sequence
  cpf1
MAPKKKRKVSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 75 - protein, 990 aa, Artificial Sequence
  CasX
MAPKKKRKVSMQEIKRINKIRRRLVKDSNTKKAGKTGPMKTLLVRVMTPDLRERLENLRKKPENIPQPISNTSRANLNKLLTDYTEMKKAILHVYWEEFQKDPVGLMSRVAQPAPKNIDQRKLIPVKDGNERLTSSGFACSQCCQPLYVYKLEQVNDKGKPHTNYFGRCNVSEHERLILLSPHKPEANDELVTYSLGKFGQRALDFYSIHVTRESNHPVKPLEQIGGNSCASGPVGKALSDACMGAVASFLTKYQDIILEHQKVIKKNEKRLANLKDIASANGLAFPKITLPPQPHTKEGIEAYNNVVAQIVIWVNLNLWQKLKIGRDEAKPLQRLKGFPSFPLVERQANEVDWWDMVCNVKKLINEKKEDGKVFWQNLAGYKRQEALLPYLSSEEDRKKGKKFARYQFGDLLLHLEKKHGEDWGKVYDEAWERIDKKVEGLSKHIKLEEERRSEDAQSKAALTDWLRAKASFVIEGLKEADKDEFCRCELKLQKWYGDLRGKPFAIEAENSILDISGFSKQYNCAFIWQKDGVKKLNLYLIINYFKGGKLRFKKIKPEAFEANRFYTVINKKSGEIVPMEVNFNFDDPNLIILPLAFGKRQGREFIWNDLLSLETGSLKLANGRVIEKTLYNRRTRQDEPALFVALTFERREVLDSSNIKPMNLIGIDRGENIPAVIALTDPEGCPLSRFKDSLGNPTHILRIGESYKEKQRTIQAAKEVEQRRAGGYSRKYASKAKNLADDMVRNTARDLLYYAVTQDAMLIFENLSRGFGRQGKRTFMAERQYTRMEDWLTAKLAYEGLPSKTYLSKTLAQYTSKTCSNCGFTITSADYDRVLEKLKKTATGWMTTINGKELKVEGQITYYNRYKRQNVVKDLSVELDRLSEESVNNDISSWTKGRSGEALSLLKKRFSHRPVQEKFVCLNCGFETHADEQAALNIARSWLFLRSQEYKKYQTNKTTGNTDKRAFVETWQSFYRKKLKEVWKPAVTS

SEQ ID NO: 76 - protein, 1069 aa, Staphylococcus aureus
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 77 - DNA, 3207 bp, Staphylococcus aureus
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 78 - DNA, 3708 bp, Artificial Sequence
  cpf1 coding sequence
atggccccaaagaagaagcggaaggtcagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac

SEQ ID NO: 79 - DNA, 2970 bp, Artificial Sequence
  CasX coding sequence
atggccccaaagaagaagcggaaggtcagcatgcaagagatcaagagaatcaacaagatcagaaggagactggtcaaggacagcaacacaaagaaggccggcaagacaggccccatgaaaaccctgctcgtcagagtgatgacccctgacctgagagagcggctggaaaacctgagaaagaagcccgagaacatccctcagcctatcagcaacaccagcagggccaacctgaacaagctgctgaccgactacaccgagatgaagaaagccatcctgcacgtgtactgggaagagttccagaaagaccccgtgggcctgatgagcagagttgctcagcccgctcctaagaacatcgaccagagaaagctgatccccgtgaaggacggcaacgagagactgacctctagcggctttgcctgcagccagtgttgccagcctctgtacgtgtacaagctggaacaagtgaacgacaagggcaagccccacaccaactacttcggcagatgcaacgtgtccgagcacgagaggctgatcctgctgtctcctcacaagcccgaggccaacgatgagctggtcacatacagcctgggcaagttcggacagagagccctggacttctacagcatccacgtgaccagggagagcaatcaccctgtgaagcccctggaacagatcggcggcaatagctgtgcctctggacctgtgggaaaagccctgagcgacgcctgtatgggagccgtggcatccttcctgaccaagtaccaggacatcatcctggaacaccagaaagtgatcaagaagaacgagaaaagactggccaacctcaaggatatcgccagcgctaacggcctggcctttcctaagatcaccctgcctccacagcctcacaccaaagagggcatcgaggcctacaacaacgtggtggcccagatcgtgatttgggtcaacctgaatctgtggcagaagctgaagatcggcagggacgaagccaagccactgcagagactgaagggcttccctagcttccctctggtggaaagacaggccaatgaagtggattggtgggacatggtctgcaacgtgaagaagctgatcaacgagaagaaagaggatggcaaggttttctggcagaacctggccggctacaagagacaagaagccctgctgccttacctgagcagcgaagaggaccggaagaagggcaagaagttcgccagataccagttcggcgacctgctgctgcacctggaaaagaagcacggcgaggactggggcaaagtgtacgatgaggcctgggagagaatcgacaagaaggtggaaggcctgagcaagcacattaagctggaagaggaaagaaggagcgaggacgcccaatctaaagccgctctgaccgattggctgagagccaaggccagctttgtgatcgagggcctgaaagaggccgacaaggacgagttctgcagatgcgagctgaagctgcagaagtggtacggcgatctgagaggcaagcccttcgccattgaggccgagaacagcatcctggacatcagcggcttcagcaagcagtacaactgcgccttcatttggcagaaagacggcgtcaagaaactgaacctgtacctgatcatcaattacttcaaaggcggcaagctgcggttcaagaagatcaaacccgaggccttcgaggctaacagattctacaccgtgatcaacaaaaagtccggcgagatcgtgcccatggaagtgaacttcaacttcgacgaccccaacctgattatcctgcctctggccttcggcaagagacagggcagagagttcatctggaacgatctgctgagcctggaaaccggctctctgaagctggccaatggcagagtgatcgagaaaaccctgtacaacaggagaaccagacaggacgagcctgctctgtttgtggccctgaccttcgagagaagagaggtgctggacagcagcaacatcaagcccatgaacctgatcggcatcgaccggggcgagaatatccctgctgtgatcgccctgacagaccctgaaggatgcccactgagcagattcaaggactccctgggcaaccctacacacatcctgagaatcggcgagagctacaaagagaagcagaggacaatccaggccgccaaagaggtggaacagagaagagccggcggatactctaggaagtacgccagcaaggccaagaatctggccgacgacatggtccgaaacaccgccagagatctgctgtactacgccgtgacacaggacgccatgctgatcttcgagaatctgagcagaggcttcggccggcagggcaagagaacctttatggccgagaggcagtacaccagaatggaagattggctcacagctaaactggcctacgagggactgcccagcaagacctacctgtccaaaacactggcccagtatacctccaagacctgcagcaattgcggcttcaccatcaccagcgccgactacgacagagtgctggaaaagctcaagaaaaccgccaccggctggatgaccaccatcaacggcaaagagctgaaggttgagggccagatcacctactacaacaggtacaagaggcagaacgtcgtgaaggatctgagcgtggaactggacagactgagcgaagagagcgtgaacaacgacatcagcagctggacaaagggcagatcaggcgaggctctgagcctgctgaagaagaggtttagccacagacctgtgcaagagaagttcgtgtgcctgaactgcggcttcgagacacacgccgatgaacaggctgccctgaacattgccagaagctggctgttcctgagaagccaagagtacaagaagtaccagaccaacaagaccaccggcaacaccgacaagagggcctttgtggaaacctggcagagcttctacagaaaaaagctgaaagaagtctggaagcccgccgtgactagt

SEQ ID NO: 80 - DNA, 3213 bp, Staphylococcus aureus
gccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 81 - DNA, 2976 bp, Campylobacter jejuni
atggccccaaagaagaagcggaaggtcgccagaatcctggccttcgacatcggcatcagcagcatcggctgggccttcagcgagaacgacgagctgaaggactgcggcgtgcggatcttcaccaaggtggaaaaccccaagaccggcgagagcctggccctgcccagaaggctggccagaagcgcccggaagagactggccagacggaaggcccggctgaaccacctgaagcacctgatcgccaacgagttcaagctgaactacgaggactaccagagcttcgacgagtccctggccaaggcctacaagggcagcctgatcagcccctacgagctgcggttccgggccctgaacgagctgctgagcaagcaggacttcgccagagtgatcctgcacattgccaagcggagaggctacgacgacatcaagaacagcgacgacaaagagaagggcgccatcctgaaggccatcaagcagaacgaggaaaagctggccaactaccagtccgtgggcgagtacctgtacaaagagtacttccagaagttcaaagagaacagcaaagaattcaccaacgtgcggaacaagaaagaaagctacgagcggtgtatcgcccagagcttcctgaaggatgagctgaagctgatcttcaagaagcagagagagttcggcttcagcttcagcaagaaattcgaggaagaggtgctgagcgtcgccttctacaagagagccctgaaggacttcagccacctcgtgggcaactgcagcttcttcaccgacgagaagagagcccccaagaacagccccctggccttcatgttcgtggccctgacccggatcatcaacctgctgaacaatctgaagaacaccgagggcatcctgtacaccaaggacgacctgaacgccctgctgaatgaggtgctgaagaacggcaccctgacctacaagcagaccaagaagctgctgggcctgagcgacgactacgagtttaagggcgagaagggcacctacttcatcgagttcaagaagtacaaagagttcatcaaggccctgggcgagcacaacctgagccaggacgatctgaatgagatcgccaaggacatcaccctgatcaaggacgagattaagctgaagaaggccctggccaaatacgacctgaatcagaaccagatcgacagcctgagcaagctggaattcaaggatcacctgaacatcagcttcaaggctctgaagctggtcacccccctgatgctggaaggcaagaagtacgacgaggcctgcaacgagctgaacctgaaggtggccatcaacgaggacaagaaggacttcctgcccgccttcaacgaaacctactacaaggacgaagtgaccaaccccgtggtgctgcgggccatcaaagaataccggaaggtgctgaatgccctgctcaagaaatacggcaaggtgcacaagatcaacatcgagctggcccgggaagtgggcaagaaccacagccagcgggccaagatcgagaaagagcagaacgaaaactacaaggccaagaaggacgctgagctggaatgcgagaagctgggactgaagatcaacagcaagaacatcctgaagctgcggctgttcaaagaacagaaagagttctgcgcctacagcggcgagaagatcaagatcagcgatctgcaggacgagaagatgctggaaatcgaccacatctacccctacagccggtccttcgacgacagctacatgaacaaggtgctggtgttcaccaaacagaaccaggaaaaactgaaccagacccccttcgaggccttcggcaacgacagcgccaagtggcagaaaatcgaggtgctggccaagaacctgcccaccaagaaacagaagagaatcctggacaagaattacaaggacaaagagcagaagaacttcaaggaccggaacctgaacgacacccggtatatcgcccggctggtgctgaactacacaaaggactacctggatttcctgcccctgtccgacgacgagaacaccaagctgaacgatacccagaaaggctccaaggtgcacgtggaagccaagagcggcatgctgaccagcgccctgagacacacctggggcttcagcgccaaggatcggaacaaccatctgcaccacgccatcgacgccgtgatcattgcctacgccaacaacagcatcgtgaaggccttctccgacttcaagaaagaacaggaaagcaacagcgccgagctgtacgccaagaagatctctgagctggactacaagaacaagcggaagttcttcgagcccttcagcggcttccggcagaaggtgctggataagatcgacgagatcttcgtgtccaagcccgagcggaagaagccctctggcgccctgcacgaggaaaccttcagaaaagaggaagagttctaccagtcctacggcggcaaagaaggcgtgctgaaggccctcgagctgggcaagatcagaaaagtgaacggcaagatcgtgaagaacggggacatgttccgggtggacatcttcaagcacaaaaagaccaacaagttctacgccgtgcccatctacaccatggacttcgccctgaaggtgctgcccaacaaggccgtggcccggtccaagaagggcgagatcaaggactggattctgatggacgagaactacgagttctgctttagcctgtacaaggactccctgatcctgatccagaccaaggacatgcaggaacccgagttcgtctactacaacgccttcaccagcagcaccgtgtccctgatcgtgtctaagcacgacaacaagttcgagacactgagcaagaaccagaagatcctgttcaagaacgccaacgagaaagaagtgatcgccaagagcatcggcatccagaatctgaaggtgttcgagaagtacatcgtgtccgccctgggagaagtgacaaaggccgagttccggcagagagaggacttcaaaaag

SEQ ID NO: 82 - DNA, 1782 bp, Artificial Sequence
  Engineered Piggybac D450N/R372A/K375A
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtggccagcaacgccagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctgaaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 83 - DNA, 1782 bp, Artificial Sequence
  Engineered Piggybac R275A/R277A
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcgccggcgcctgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 84 - DNA, 1782 bp, Artificial Sequence
  Engineered Piggybac K409A/K412A
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccgcccccgccgccatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 85 - DNA, 1782 bp, Artificial Sequence
  Engineered Piggybac R460A/K461A
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcgccgccaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 86 - DNA, 1782 bp, Artificial Sequence
  Engineered Piggybac S351A/R372A/K375A/R388A/ D450N/W465A/S573A/M589V/S594L
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccgccatccccctggccaagaacctgctgcaggagccctacaagctgaccatcgtgggcaccgtggccagcaacgccagagagatccccgaggtcctgaagaacagcaggtccgcccccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctgaaccagatgtgcagcgtgatgacctgcagcagaaagaccaacagggcccccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccgcctgcaagaagtgtaagaaggtcatctgccgggagcacaacatcgacgtgtgccagaggtgtctc

SEQ ID NO: 87 - DNA, 1782 bp, Artificial Sequence
  Engineered Piggybac R275A/R277A/N347S/K375A/T560A/S573A/M589V/S594L
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcgccggcgcctgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccgcccccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctgaaccagatgtgcagcgtgatgacctgcagcagaaagaccaacagggcccccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccgcctgcaagaagtgtaagaaggtcatctgccgggagcacaacatcgacgtgtgccagagctgtctc

SEQ ID NO: 88 - DNA, 1133 bp, Artificial Sequence
  engineered PiggyBac R245A/R275A/R277A/R372A/W465A
tcgacttcctgatcagatgcctgaggatggacgacaagagcatcaggcccaccctgcgggagaacgacgtgttcacccccgtggccaagatctgggacctgttcatccaccagtgcatccagaactacacccctggcgcccacctgaccatcgacgagcagctgctgggcttcgccggcgcctgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtggccagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacagggcccccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 89 - DNA, 1782 bp, Artificial Sequence
  engineered Piggybac R372A/K375A/R388A/D450N/W465A/S573A/M589V/S594L
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtggccagcaacgccagagagatccccgaggtcctgaagaacagcaggtccgcccccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctgaaccagatgtgcagcgtgatgacctgcagcagaaagaccaacagggcccccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccgcctgcaagaagtgtaagaaggtcatctgccgggagcacaacatcgacgtgtgccagaggtgtctc

SEQ ID NO: 90 - protein, 594 aa, Artificial Sequence
  Engineered Piggybac M194A
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHASTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 91 - protein, 594 aa, Artificial Sequence
  Engineered Piggybac R245A
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVAKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 92 - protein, 594 aa, Artificial Sequence
  Engineered Piggybac R325A
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLAEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 93 - protein, 594 aa, Artificial Sequence
  Engineered Piggybac R372A
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 94 - protein, 594 aa, Artificial Sequence
  Engineered Piggybac R375A
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNAREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 95 - protein, 594 aa, Artificial Sequence
  Engineered Piggybac R376A
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKAEIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 96 - protein, 594 aa, Artificial Sequence
  Engineered Piggybac E377A
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKRAIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 97 - protein, 594 aa, Artificial Sequence
  Engineered Piggybac E380A
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPAVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 98 - protein, 594 aa, Artificial Sequence
  Engineered Piggybac D450N
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 99 - protein, 594 aa, Artificial Sequence
  Engineered Piggybac S564P
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPPKIRRKASASCKKCKKVICREHNIDMCQSCF

SEQ ID NO: 100 - DNA, 1782 bp, Artificial Sequence
  engineered Piggybac
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacgccagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 101 - DNA, 1782 bp, Artificial Sequence
  engineered Piggybac
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtggccaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 102 - DNA, 1782 bp, Artificial Sequence
  engineered Piggybac
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctggccgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 103 - DNA, 1782 bp, Artificial Sequence
  engineered Piggybac
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtggccagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 104 - DNA, 1782 bp, Artificial Sequence
  Engineered Piggybac R375A
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacgccagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 105 - DNA, 1782 bp, Artificial Sequence
  Engineered Piggybac R376A
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaaggccgagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 106 - DNA, 1782 bp, Artificial Sequence
  Engineered Piggybac E377A
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagccatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 107 - DNA, 1782 bp, Artificial Sequence
  Engineered Piggybac E380A
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgccgtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 108 - DNA, 1782 bp, Artificial Sequence
  Engineered Piggybac D450N
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctgaaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 109 - DNA, 1782 bp, Artificial Sequence
  Engineered Piggybac S564P
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtccccccaagatcagaagaaaggccagcgccagctgcaagaagtgtaagaaggtcatctgccgggagcacaacatcgacatgtgccagagctgtttc

SEQ ID NO: 110 - DNA, 5931 bp, Artificial Sequence
  Engineered Piggybac S351A/R372A/K375A/R388A/D450N/W465A/S573A/M589V/592G/S594L
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagattccggtagcgaaacaccggggacttcagaatcggccaccccggagtctggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccgccatccccctggccaagaacctgctgcaggagccctacaagctgaccatcgtgggcaccgtggccagcaacgccagagagatccccgaggtcctgaagaacagcaggtccgcccccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctgaaccagatgtgcagcgtgatgacctgcagcagaaagaccaacagggcccccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccgcctgcaagaagtgtaagaaggtcatctgccgggagcacaacatcgacgtgtgccagggctgtttg

SEQ ID NO: 111 - DNA, 5931 bp, Artificial Sequence
  Engineered Piggybac R245A/R275A/R277A/R372A/W465A/M589V
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagattccggtagcgaaacaccggggacttcagaatcggccaccccggagtctggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccgccatccccctggccaagaacctgctgcaggagccctacaagctgaccatcgtgggcaccgtggccagcaacgccagagagatccccgaggtcctgaagaacagcaggtccgcccccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctgaaccagatgtgcagcgtgatgacctgcagcagaaagaccaacagggcccccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtccccccaagatcagaagaaaggccagcgccgcctgcaagaagtgtaagaaggtcatctgccgggagcacaacatcgacatgtgccagaggtgtctc

SEQ ID NO: 112 - DNA, 5931 bp, Artificial Sequence
  Engineered Piggybac R275A/325A/R372A/T560A
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagattccggtagcgaaacaccggggacttcagaatcggccaccccggagtctggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcgccggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctggccgagtactacgtgaaggagctgtccaagcccgtccacggcagctgcagaaacatcacctgcgacaactggttcaccagcatccccctggccaagaacctgctgcaggagccctacaagctgaccatcgtgggcaccgtggccagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctacgcctactactgtcccagcaagatcagaagaaaggccagcgccagctgcaagaagtgtaagaaggtcatctgccgggagcacaacatcgacatgtgccagagctgtttc

SEQ ID NO: 113 - DNA, 20 bp, Artificial Sequence
  gRNA
tatgtacacttctgacccac

SEQ ID NO: 114 - DNA, 20 bp, Artificial Sequence
  gRNA SaCas9
gtatcacaattccagtgggt

SEQ ID NO: 115 - DNA, 20 bp, Artificial Sequence
  gRNA SaCas9
gtatcacaattccagtgggt

SEQ ID NO: 116 - DNA, 20 bp, Artificial Sequence
  gRNA SaCas9
ggacaggatcggcataaccg

SEQ ID NO: 117 - DNA, 20 bp, Artificial Sequence
  gRNA SaCas9
gtgctcggggccactaggga

SEQ ID NO: 118 - DNA, 20 bp, Artificial Sequence
  Cpf1 gRNA
acttataattcactgtatca

SEQ ID NO: 119 - DNA, 20 bp, Artificial Sequence
  Cpf1 gRNA
agcttgatatccatggaatt

SEQ ID NO: 120 - DNA, 20 bp, Artificial Sequence
  Cpf1 gRNA
tgctcggggccactagggac

SEQ ID NO: 121 - DNA, 20 bp, Artificial Sequence
  Cpf1 gRNA
cttttgtaaaactttatggt

SEQ ID NO: 122 - DNA, 20 bp, Artificial Sequence
  Cpf1 gRNA
caaaagtaaatagcccggct

SEQ ID NO: 123 - DNA, 22 bp, Artificial Sequence
  CjCas9
gccgatcctgtccctagtggcc

SEQ ID NO: 124 - DNA, 22 bp, Artificial Sequence
  CjCas9 gRNA
acaattccagtgggtcagaagt

SEQ ID NO: 125 - DNA, 21 bp, Artificial Sequence
  CjCas9 gRNA
acacttctgacccactggaat

SEQ ID NO: 126 - DNA, 22 bp, Artificial Sequence
  CjCas9 gRNA
gaattccatggatatcaagctt

SEQ ID NO: 127 - DNA, 22 bp, Artificial Sequence
  CjCas9 gRNA
aattccagtgggtcagaagtgt

SEQ ID NO: 128 - DNA, 20 bp, Artificial Sequence
  CasX gRNA
tcaagcgcgtgtatgtacac

SEQ ID NO: 129 - DNA, 20 bp, Artificial Sequence
  CasX gRNA
ggatcggcataaccggtgaa

SEQ ID NO: 130 - DNA, 20 bp, Artificial Sequence
  CasX gRNA
tagacatgaggtctatggac

SEQ ID NO: 131 - DNA, 20 bp, Artificial Sequence
  CasX gRNA
taagcttgatatccatggaa

SEQ ID NO: 132 - DNA, 20 bp, Artificial Sequence
  CasX gRNA
tataattcactgtatcacaa

SEQ ID NO: 133 - protein, 5 aa, Artificial sequence
  SITE 1..5: wherein the sequence can be repeated 1 to 50 times
  Linker
GGGGS

SEQ ID NO: 134 - protein, 5 aa, Artificial sequence
  SITE 1..5: wherein the sequence can be repeated 1 to 50 times
  linker
EAAAK

SEQ ID NO: 135 - protein, 594 aa, Artificial Sequence
  Modified hyperactive PiggyBac R275A/N347S/K375A/D450N/S592G
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFAGRCPFRVYIPNKPSKYGIKILMMCDSGTKYMINGMPYLGRGTQTNGVPLGEYYVKELSKPVHGSCRNITCDSWFTSIPLAKNLLQEPYKLTIVGTVRSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQGCF

SEQ ID NO: 136 - protein, 594 aa, Artificial Sequence
  Modified hyperactive PiggyBac N347S/D450N/T560A/S573A/F594L
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDSWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPSKIRRKASAACKKCKKVICREHNIDMCQSCL

SEQ ID NO: 137 - protein, 594 aa, Artificial Sequence
  Modified hyperactive PiggyBac N347A/D450N
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDSWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 138 - protein, 594 aa, Artificial Sequence
  Modified hyperactive PiggyBac R275A/N347S/R372A/D450N/T560A/F594L
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFAGRCPFRVYIPNKPSKYGIKILMMCDSGTKYMINGMPYLGRGTQTNGVPLGEYYVKELSKPVHGSCRNITCDSWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPSKIRRKASASCKKCKKVICREHNIDMCQSCL

SEQ ID NO: 139 - protein, 594 aa, Artificial Sequence
  Modified hyperactive PiggyBac R202K/R275A/N347S/R372A/D450N/T560A/F594L
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDKSLSMVYVSVMSRDRFDFLIRCLRMDDKSIRPTLRENDVFTPVRKIWDLFIHQCIQNYTPGAHLTIDEQLLGFAGRCPFRVYIPNKPSKYGIKILMMCDSGTKYMINGMPYLGRGTQTNGVPLGEYYVKELSKPVHGSCRNITCDSWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPSKIRRKASASCKKCKKVICREHNIDMCQSCL

SEQ ID NO: 140 - protein, 594 aa, Artificial Sequence
  Modified hyperactive PiggyBac R275A/R277A/N347S/R372A/D450N/T560A/S564P/F594L
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFAGACPFRVYIPNKPSKYGIKILMMCDSGTKYMINGMPYLGRGTQTNGVPLGEYYVKELSKPVHGSCRNITCDSWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPPKIRRKASASCKKCKKVICREHNIDMCQSCL

SEQ ID NO: 141 - protein, 594 aa, Artificial Sequence
  Modified Hyperactive Piggybac triple mutant D450N/R372A/K375A/R376A
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAAEIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 142 - protein, 594 aa, Artificial Sequence
  Modified hyperactive PiggyBac R245A/N347S/R372A/D450N/T560A/S564P/S573A/S592G
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVAKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDSWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPPKIRRKASAACKKCKKVICREHNIDMCQGCF

SEQ ID NO: 143 - protein, 594 aa, Artificial Sequence
  Modified hyperactive PiggyBac R277A/G325A/N347A/K375A/D450N/T560A/S564P/S573A/S592G/F594L
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGACPFRVYIPNKPSKYGIKILMMCDSGTKYMINGMPYLGRGTQTNGVPLAEYYVKELSKPVHGSCRNITCDAWFTSIPLAKNLLQEPYKLTIVGTVRSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPPKIRRKASAACKKCKKVICREHNIDMCQGCL

SEQ ID NO: 144 - protein, 594 aa, Artificial Sequence
  Modified hyperactive PiggyBac V34M/R275A/G325A/N347S/S351A/R372A/K375A/D450N/T560A/S564P
MGSSLDDEHILSALLQSDDELVGEDSDSEVSDHMSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFAGRCPFRVYIPNKPSKYGIKILMMCDSGTKYMINGMPYLGRGTQTNGVPLAEYYVKELSKPVHGSCRNITCDSWFTAIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPPKIRRKASASCKKCKKVICREHNIDMCQSCF

SEQ ID NO: 145 - protein, 594 aa, Artificial Sequence
  Modified hyperactive PiggyBac G325A/N347S/K375A/D450N/S573A/M589V/S592G
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLAEYYVKELSKPVHGSCRNITCDSWFTSIPLAKNLLQEPYKLTIVGTVRSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASAACKKCKKVICREHNIDVCQGCF

SEQ ID NO: 146 - protein, 594 aa, Artificial Sequence
  Modified hyperactive PiggyBac S230N/R277A/N347S/K375A/D450N
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKNIRPTLRENDVFTPVRKIWDLFIHQCIQNYTPGAHLTIDEQLLGFRGACPFRVYIPNKPSKYGIKILMMCDSGTKYMINGMPYLGRGTQTNGVPLGEYYVKELSKPVHGSCRNITCDSWFTSIPLAKNLLQEPYKLTIVGTVRSNKREIPEVLANSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 147 - protein, 594 aa, Artificial Sequence
  Modified hyperactive PiggyBac T43I/R372A/K375A/A411T/D450N
MGSSLDDEHILSALLQSDDELVGEDSDSEVSDHVSEDDVQSDIEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPTKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 148 - protein, 594 aa, Artificial Sequence
  Modified hyperactive PiggyBac G325A/N347S/S351A/K375A/D450N/S573A/M589V/S592G
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLAEYYVKELSKPVHGSCRNITCDSWFTAIPLAKNLLQEPYKLTIVGTVRSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASAACKKCKKVICREHNIDVCQGCF

SEQ ID NO: 149 - protein, 594 aa, Artificial Sequence
  Modified hyperactive PiggyBac Y177H/R275A/G325A/K375A/D450N/T560A/S564P/S592G
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIHAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFAGRCPFRVYIPNKPSKYGIKILMMCDSGTKYMINGMPYLGRGTQTNGVPLAEYYVKELSKPVHGSCRNITCDNWFTSIPLAKNLLQEPYKLTIVGTVRSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPGKIRRKASASCKKCKKVICREHNIDMCQGCF

SEQ ID NO: 150 - DNA, 390 bp, Artificial Sequence
  MCP N55K
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctac

SEQ ID NO: 151 - protein, 130 aa, Artificial Sequence
  MCP N55K
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 152 - DNA, 162 bp, Artificial Sequence
  gRNA-MS2(tetraloop)-AAVS1-3 spacer
ggggccactagggacaggatgttttagagctaggccaacatgaggatcacccatgtctgcagggcctagcaagttaaaataaggctagtccgttatcaacttggccaacatgaggatcacccatgtctgcagggccaagtggcaccgagtcggtgctttttt

SEQ ID NO: 153 - RNA, 162 nt, Artificial Sequence
  gRNA-MS2(tetraloop)-AAVS1-3 spacer
ggggccacuagggacaggauguuuuagagcuaggccaacaugaggaucacccaugucugcagggccuagcaaguuaaaauaaggcuaguccguuaucaacuuggccaacaugaggaucacccaugucugcagggccaaguggcaccgagucggugcuuuuuu